(19) United States
(12) Patent Application Publication
(10) Pub. No.: US 2026/0357233 A1
(43) Pub. Date: Nov. 12, 2026

(54) ENDONUCLEASE SYSTEMS

(71) Applicant: Metagenomi Therapeutics, Inc., Emeryville, CA (US)

(72) Inventors: Brian C. Thomas, Berkeley, CA (US); Lisa Alexander, Berkeley, CA (US); Ian Barr, Albany, CA (US); Christopher Brown, Albany, CA (US); Daniela S.A. Goltsman, Oakland, CA (US); Samatar Jirde, Emeryville, CA (US); Isabel Nocedal, Oakland, CA (US)

(21) Appl. No.: 19/486,289

(22) PCT Filed: May 23, 2024
(86) PCT No.: PCT/US2024/030874

§ 371 Date: Nov. 20, 2025

Related U.S. Application Data
(60) Provisional application No. 63/503,927, filed on May 23, 2023.
(60) Provisional application No. 63/520,864, filed on Aug. 21, 2023.

(51) Int. Cl. C12N 9/22; C12N 15/11

(52) U.S. Cl. CPC C12N 9/226; C12N 15/11; C12N 2310/20

(57) ABSTRACT

The present disclosure provides endonuclease enzymes as well as methods of using such enzymes or variants thereof.

### CROSS-REFERENCE TO RELATED APPLICATIONS

This application is the U.S. National Stage entry of International Application No. PCT/US2024/030874, filed on May 23, 2024, which claims the benefit of and priority to U.S. Provisional Application No. 63/503,927 filed on May 23, 2023, and U.S. Provisional Application No. 63/520,864 filed on Aug. 21, 2023, the entire disclosure of each of which is incorporated by reference in its entirety herein for all purposes.

### SEQUENCE LISTING

This instant application contains a Sequence Listing which has been submitted electronically in XML format and is hereby incorporated by reference in its entirety. Said XML copy, created on May 23, 2024, is named MTG-027WO_SL.xml and is 3,611,069 bytes in size.

### SUMMARY

The large size (greater than 1200 amino acids (aa)) of many class 2 Cas effectors makes delivery for therapeutic applications challenging. Accordingly, described herein are methods, compositions, and systems relating to putative guided dsDNA nucleases referred to as SMART (SMall ARchaeal-associaTed) nuclease systems. These endonuclease effectors are defined by their small size (about 400 aa to about 1050 aa) and the presence of RuvC and HNH catalytic domains.

Described herein, in certain embodiments, are engineered nuclease systems comprising: an endonuclease comprising a sequence having at least 80% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 80% identity to any one of SEQ ID NOs: 1350-1368 and 1415-1440. In some embodiments, the endonuclease comprises a sequence having at least 80% identity to any one of SEQ ID NOs: 1324 and 1329-1346.

Described herein, in certain embodiments, are engineered nuclease systems comprising: an endonuclease comprising a sequence having at least 90% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 90% identity to 1350-1368 and 1415-1440. In some embodiments, the endonuclease comprises a sequence having at least 90% identity to any one of SEQ ID NOs: 1324 and 1329-1346.

Described herein, in certain embodiments, are engineered nuclease systems comprising an endonuclease comprising a sequence having at least 95% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 95% identity to 1350-1368 and 1415-1440. In some embodiments, the endonuclease comprises a sequence having at least 95% identity to any one of SEQ ID NOs: 1324 and 1329-1346.

Described herein, in certain embodiments, are engineered nuclease systems comprising an endonuclease comprising a sequence having at least 99% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 99% identity to 1350-1368 and 1415-1440. In some embodiments, the endonuclease comprises a sequence having at least 99% identity to any one of SEQ ID NOs: 1324 and 1329-1346.

Described herein, in certain embodiments, are engineered nuclease systems comprising an endonuclease comprising a sequence having 100% sequence identity to any one of SEQ ID NOs: 1323-1324, 1329-1347, 1350-1368, and 1415-1440; and an engineered guide polynucleotide that forms a complex with the endonuclease and hybridizes to a target nucleic acid sequence. In some embodiments, the endonuclease comprises a sequence having 100% sequence identity to any one of SEQ ID NOs: 1347, 1350-1368, and 1415-1440. In some embodiments, the endonuclease comprises a sequence having 100% identity to any one of SEQ ID NOs: 1323, 1324 and 1329-1346.

In some embodiments, the engineered guide polynucleotide is a single guide nucleic acid. In some embodiments, the engineered guide polynucleotide is a dual guide nucleic acid. In some embodiments, the engineered guide polynucleotide is RNA. In some embodiments, the endonuclease binds non-covalently to the engineered guide polynucleotide. In some embodiments, the endonuclease is covalently linked to the engineered guide polynucleotide. In some embodiments, the endonuclease is fused to the engineered guide polynucleotide.

In some embodiments, the engineered guide polynucleotide comprises a sequence having at least 90% sequence identity to any one of SEQ ID NOs: 1327-1328, 1348, 1369-1372, 1376-1391, 1392-1414, and 1470-2242.

In some embodiments, the engineered guide polynucleotide comprises a sequence having at least 90% sequence identity to any one of SEQ ID NOs: 1571, 1591, 1592, 1615, 1625, 1651, 1663, 1672, 1709, 1712, 1713, 1728, 1738, 1764, 1809, 1812, 1884, 1821, 1853, 1893, 1846, 1854, 1878, 1886, 1902, 1890, 1847, 1903, 1890, 1957, 1959, 1960, 1961, 1975, 1988, and 2002. In some embodiments, the engineered guide polynucleotide comprises a sequence having at least 90% sequence identity to SEQ ID NOs: 1410 or 1960.

In some embodiments, the engineered guide polynucleotide comprises a sequence having at least 90% sequence identity to any one of SEQ ID NOs: 1410, 1412, 1953, 1956, 1960, 1961, 1966, 1970, and 1478.

In some embodiments, the engineered guide polynucleotide comprises a sequence having at least 90% sequence identity to any one of SEQ ID NOs: 2157, 2159, and 2160.

In some embodiments, the engineered guide polynucleotide comprises a sequence having at least 90% sequence identity to any one of SEQ ID NOs: 2017, 2022, 2029, 2031, 2032, 2035, 2044, 2045, 2047, 2048, 2073, 2075, 2090, 2195, 2197, 2198, 2199, 2200, and 2202.

In some embodiments, the engineered guide polynucleotide comprises a sequence having at least 90% sequence identity to any one of SEQ ID NOs: 2017, 2022, 2026, 2028, 2029, 2031, 2032, 2035, 2044, 2047, 2054, 2073, 2075, 2090, 2195, 2197, 2198, 2199, 2200, 2202, 2206, 2208, 2211, 2212, and 2216.

In some embodiments, the engineered guide polynucleotide comprises a sequence having 100% sequence identity to any one of SEQ ID NOs: 1327-1328, 1348, 1369-1372, 1376-1391, 1392-1414, and 1470-2242.

Described herein, in certain embodiments, are methods for modifying a target nucleic acid sequence comprising contacting the target nucleic acid sequence using the engineered nuclease system described herein. In some embodiments, modifying the target nucleic acid sequence comprises binding, nicking, or cleaving, the target nucleic acid sequence. In some embodiments, the target nucleic acid sequence comprises genomic DNA, viral DNA, viral RNA, or bacterial DNA. In some embodiments, the modification is in vitro. In some embodiments, the modification is in vivo. In some embodiments, the modification is ex vivo.

Described herein, in certain embodiments, are methods of modifying a target nucleic acid sequence in a mammalian cell comprising contacting the mammalian cell using the engineered nuclease system described herein. In some embodiments, the methods further comprise selecting cells comprising the modification.

Described herein, in certain embodiments, are cells comprising the engineered nuclease system described herein. In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell. In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof. In some embodiments, the cell is an engineered cell. In some embodiments, the cell is a stable cell.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIGS. 1A-FIG. 1D depict phylogenetic trees of SMART I nucleases for ancestral reconstruction of the MG34 (FIG. 1A-1C) and MG102 (FIG. 1D) clades. Phylogenetic trees were inferred with FastTree or RAxML from MAFFT global (G-INS-i) or local (L-INS-i) multiple sequence alignments. Individual ancestors indicated for each tree by a circle represent different ancestral ages and are based on different phylogenetic trees. FIG. 1D shows previously characterized effectors MG102-2, MG102-39, and MG102-42 in the tree.

FIGS. 2A-2B depict that novel SMART I effectors are active nucleases with non-native guides. SMART I effectors were assayed for cleavage activity via a PAM enrichment protocol. FIG. 2A depicts PCR results. Effectors were expressed in in-vitro transcription/translation (IVTT) reactions in the presence of the single guide RNA from other active MG34 nucleases and added to a PAM library (dsDNA target). Cleavage products were amplified via ligation to the cut site and subsequent PCR amplification. Successful RNA guided cleavage by the nuclease produced bands at the expected 180 bp size. FIG. 2B depicts NGS sequencing results. NGS sequencing of the bands identified in FIG. 2A were used to generate the PAM sequence and cleavage position relative to the PAM sequence.

FIGS. 3A-FIG. 3B depict that that novel SMART I effectors are active nucleases. SMART I ancestral effectors were assayed for cleavage activity via a PAM enrichment protocol. FIG. 3A depicts PCR results. Effectors were expressed in in-vitro transcription/translation (IVTT) reactions in the presence of the single guide RNA from other active MG34 nucleases and added to a PAM library (dsDNA target). Cleavage products were amplified via ligation to the cut site and subsequent PCR amplification. Successful RNA guided cleavage by the nuclease produced bands at the expected 180 bp size. FIG. 3B depicts NGS sequencing results. NGS sequencing of the bands identified in FIG. 3A were used to generate the PAM sequence and cleavage position relative to the PAM sequence.

FIG. 4A-FIG. 4B illustrates that the novel chimeric SMART I ancestral effectors are active nucleases. Chimeric SMART I ancestral effectors were assayed for cleavage activity via a PAM enrichment protocol. FIG. 4A depicts PCR results. Effectors were expressed in in-vitro transcription/translation (IVTT) reactions in the presence of the single guide RNA from other active MG34 nucleases and added to a PAM library (dsDNA target). Cleavage products were amplified via ligation to the cut site and subsequent PCR amplification. Successful RNA guided cleavage by the nuclease produced bands at the expected 180 bp size. FIG. 4B shows NGS sequencing results. NGS sequencing of the bands identified in FIG. 4A for MG34-47 were used to generate the PAM sequence and cleavage position relative to the PAM sequence.

FIG. 5 depicts bar plots showing SMART I ancestral effectors recognize diverse PAM sequences. MG34 nucleases were quantitatively assayed for cleavage activity via an in-vitro cleavage assay. The effectors were expressed in in-vitro transcription/translation (IVTT) reactions in the absence (apo) or presence of the single guide RNA from other active MG34 nucleases, and dsDNA target with nRR PAMs (nAA, nAG, nGA, or nGG PAMs) were incubated with ribonucleoprotein complexes to initiate cleavage. Cleavage products were analyzed by nucleic acid electrophoresis and peak area for uncleaved (~3500 bp supercoiled) and cleavage products (~2200 bp linearized) were plotted as a percent of RNA-guided cleavage (y axis).

FIGS. 6A-FIG. 6B depict SDS page gel results showing that SMART I ancestral effectors are soluble. FIG. 6A shows purification of MG34-27 and FIG. 6B shows purification of MG34-29. Samples with 2× Laemmli buffer were separated on a Stain-Free 4-20% gradient SDS PAGE gel and visualized by fluorescence imaging. In each, the “Sonication” lanes show the whole cell sample after lysis, “Load” shows the contents of the soluble portion of the cell lysate. In FIG. 6A, the lanes A11-B10 show the eluate from a gradient elution from 20-300 mM Imidazole. In FIG. 6B, we see the results of a step elution done at the three different pH levels.

FIGS. 7A-FIG. 7B depict activity assay results showing RNA-directed cleavage of plasmids by MG34-27 (FIG. 7A) and MG34-29 (FIG. 7B) with sgRNAs from extant nucleases. MG34-29 (50 nM) was complexed with excess MG34-1 sgRNA (sg1) and used to cleave a supercoiled plasmid containing an nGG PAM. MG34-27 was complexed with sgRNAs from MG34-1 and MG34-25 (sg1 and sg25) and used to cleave four separate PAM containing plasmids. The expected size of the cleaved plasmids is 2200 bp, and the products are indicated by the red oval.

FIG. 8 depicts the results of a nuclease activity assay. Nuclease activity was tested by nucleofecting MG34-27 or MG34-29 mRNA (500 ng) and sgRNA (200 pmol) targeting individual sites in the AAVS1 locus into K562 cells. After 72 hours, cells were harvested and prepped for NGS to assess editing efficiency. Each bar represents editing efficiency at the site targeted by the specific spacer (X axis) and is the average of two replicates.

FIG. 9 depicts the results of a nuclease activity assay. Nuclease activity was tested by nucleofecting MG34-29 mRNA (500 ng) and increasing amounts of sgRNA (100, 200, 300, or 400 pmol) targeting individual sites in the AAVS1 locus into K562 cells. After 72 hours, cells were harvested and NGS libraries were prepared to assess editing efficiency. Each bar represents editing efficiency at the site targeted by the specific spacer (C7, E7, F7 or G7) and is the average of two replicates.

FIGS. 10A-10C depict the identification of ancestral sequences. Ancestral intermediate sequences were generated based on a phylogenetic tree which included 441 sequences (FIG. 10A) or 190 sequences (FIG. 10B). Ancestral intermediate sequences were generated by randomly introducing amino acids from one ancestor (First) into a second ancestor using the weights shown in FIG. 10C, as well as the PAML probabilities derived from the original ancestral sequence reconstruction. Up to four different groups of ancestors were generated for the two trees based on ancestral nodes 1 through 4 and the relative weights for each ancestor in the random sampling as given by the “Sampling weight” columns in FIG. 10C.

FIG. 11A depicts a cleavage activity assay. MG34 SMART I effectors were assayed for cleavage activity via a PAM enrichment protocol. Effectors were expressed in in-vitro transcription/translation (IVTT) reactions in the presence of the single guide RNA from the active nuclease MG34-1 and added to a PAM library (dsDNA target). Cleavage products were amplified via ligation to the cut site and subsequent PCR amplification. Successful RNA-guided cleavage by the nuclease produced bands at the expected 180 bp size (arrows). The positive control represents the MG34-1 nuclease with its native guide RNA, while lane numbers represent the MG34 candidate tested (e.g. lane 55 is MG34-55).

FIG. 11B depicts a cleavage activity assay. SMART I effectors were assayed for cleavage activity via a PAM enrichment protocol. Effectors were expressed in in-vitro transcription/translation (IVTT) reactions in the presence of the single guide RNA from the active nuclease MG34-25 and added to a PAM library (dsDNA target). Cleavage products were amplified via ligation to the cut site and subsequent PCR amplification. Successful RNA-guided cleavage by the nuclease produced bands at the expected 180 bp size. Lane numbers represent the MG34 candidate tested (e.g. lane 55 is MG34-55).

FIG. 12 depicts NGS sequencing of the bands identified in FIGS. 11A-11B, which were used to generate the preferred PAM sequence and cleavage position relative to the PAM sequence for the active nucleases MG34-71, MG34-72, MG34-73, and MG34-74.

FIG. 13 depicts NGS sequencing of the bands identified in FIG. 11 were used to generate the preferred PAM sequence and cleavage position relative to the PAM sequence for the active nucleases MG34-75, MG34-76, MG34-77, MG34-78, and MG34-79.

FIG. 14 depicts a cleavage activity assay. MG34-35 effector was assayed for cleavage activity via a PAM enrichment protocol. Effectors were expressed in in-vitro transcription/translation (IVTT) reactions in the presence of a single guide RNA (SEQ ID NOs: 1392-1399) and added to a PAM library (dsDNA target). Cleavage products were amplified via ligation to the cut site and subsequent PCR amplification. Successful RNA-guided cleavage by the nuclease produced bands at the expected 180 bp size.

FIG. 15 depicts a cleavage activity assay. MG102 SMART I effectors were assayed for cleavage activity via a PAM enrichment protocol. Effectors were expressed in in-vitro transcription/translation (IVTT) reactions in the presence of a single guide RNA and added to a PAM library (dsDNA target). For native effectors (51, 53, 55, and 63) four single guide designs were tested, and for ancestral effectors (64-81), the guides from active candidates MG102-2, MG102-39, and MG102-42 were used. Cleavage products were amplified via ligation to the cut site and subsequent PCR amplification. Successful RNA-guided cleavage by the nuclease produced bands at the expected 180 bp size.

FIGS. 16A-16E depict NGS sequencing of the bands identified in FIG. 15, which were used to generate the preferred PAM sequence (FIG. 16A) and cleavage position relative to the PAM sequence for the active nucleases MG102-51, MG102-53, MG102-55, MG102-63, MG102-65, MG102-66, MG102-67, MG102-68, MG102-71, MG102-73, MG102-74, MG102-77, MG102-78, MG102-79, MG102-80 (FIGS. 16B-16E).

FIG. 17 depicts MG34-29 mammalian activity at Beta-2 microglobulin (B2M) locus with MG34-1 nGG PAM guides. Nuclease activity was tested by nucleofecting MG34-29 mRNA (500 ng) and sgRNA (200 pmol) targeting individual sites in the B2M locus into K562 cells using an MG34-1 guide scaffold. 192 B2M guides were tested. After 72 hours, cells were harvested and prepped for NGS to assess editing efficiency. Each bar represents editing efficiency at the site targeted by the specific spacer (X axis) and is the average of three replicates.

FIG. 18 depicts MG34-29 mammalian activity at hRosa26 locus with MG34-1 nGG PAM guides. Nuclease activity was tested by nucleofecting MG34-29 mRNA (500 ng) and sgRNA (200 pmol) targeting individual sites in the hRosa26 locus into K562 cells using an MG34-1 guide scaffold. 72 hRosa26 guides were tested. After 72 hours, cells were harvested and prepped for NGS to assess editing efficiency. Each bar represents editing efficiency at the site targeted by the specific spacer (X axis) and is the average of three replicates.

FIG. 19 depicts ASR MG34-29 mammalian activity at AAVS1 locus with MG34-1 nGG PAM guides. Nuclease activity was tested by nucleofecting MG34-29 mRNA (500 ng) and sgRNA (200 pmol) targeting individual sites in the AAVS1 locus into K562 cells using an MG34-1 guide scaffold. 192 B2M guides were tested in this study. 96 AAVS1 guides were tested. After 72 hours, cells were harvested and prepped for NGS to assess editing efficiency. Each bar represents editing efficiency at the site targeted by the specific spacer (X axis) and is the average of three replicates.

FIG. 20 depicts ASR MG34-29 mammalian activity with a MG34-1 ABE active guide targeting EMX1. Nuclease activity was tested by nucleofecting MG34-29 mRNA (500 ng) and sgRNA (100, 200, 300, and 400 pmol) targeting individual sites in the AAVS1 (pPE guide) into K562 cells using an MG34-1 guide scaffold. 4 varying locus guides were tested. After 72 hours, cells were harvested and prepped for NGS to assess editing efficiency. Each bar represents editing efficiency at the site targeted by the specific spacer (X axis).

FIG. 21 depicts ASR MG34-29 mammalian activity at AAVS1 with MG34-25 scaffold nGG PAM guides. Nuclease activity was tested by nucleofecting MG34-29 mRNA (500 ng) and sgRNA (200 pmol) targeting individual sites in the AAVS1 locus into K562 cells using an MG34-25 guide scaffold. 96 AAVS1 guides were tested. After 72 hours, cells were harvested and prepped for NGS to assess editing efficiency. Each bar represents editing efficiency at the site targeted by the specific spacer (X axis) and is the average of two replicates.

FIG. 22 depicts ASR MG34s mammalian activity with 4 AAVS1 locus MG34-1 and 4 MG34-25 scaffold active spacers. Nuclease activity was tested by nucleofecting various MG34 nuclease mRNAs (500 ng) and sgRNA (200 pmol) targeting 4 individual sites in the AAVS1 locus into K562 cells, using MG34-1 (left) or MG34-25 (right) guide scaffolds. 4 AAVS1 guides were tested. After 72 hours, cells were harvested and NGS libraries were prepared to assess editing efficiency. Each bar represents editing efficiency at the site targeted by the specific spacer (C7, E7, F7 or G7) and guide scaffold combination.

FIG. 23 depicts ASR MG34-72 mammalian activity at AAVS1 with MG34-25 scaffold nGG PAM guides. Nuclease activity was tested by nucleofecting MG34-72 mRNA (500 ng) and sgRNA (200 pmol) targeting individual sites in the AAVS1 locus into K562 cells using an MG34-25 guide scaffold. 96 AAVS1 guides were tested. After 72 hours, cells were harvested and prepped for NGS to assess editing efficiency. Each bar represents editing efficiency at the site targeted by the specific spacer (X axis) and is the average of two replicates.

FIG. 24 depicts MG102 mammalian activity with MG102-53 nAR AAVS1 guides. Nuclease activity was tested by nucleofecting MG102-53 (500 ng) with its own nAR PAM sgRNAs (450 pmol), targeting individual sites in the AAVS1 locus into K562 cells. 96 AAVS1 guides were tested. After 72 hours, cells were harvested and NGS libraries were prepared to assess editing efficiency. Each bar represents editing efficiency at the site targeted by the specific spacer (x axis).

FIG. 25 depicts ASR MG102-68 mammalian activity with MG102-39 nRC PAM guides. Nuclease activity was tested by nucleofecting MG102-68 mRNA (500 ng) with sgRNA (450 pmol) targeting individual sites in the AAVS1 locus into K562 cells, using the MG102-39 guide scaffold at nRC PAM sites. 96 AAVS1 guides were tested. After 72 hours, cells were harvested and NGS libraries were prepared to assess editing efficiency. Each bar represents editing efficiency at the site targeted by the specific spacer (x axis).

FIG. 26 depicts ASR MG102 mammalian activity with MG102-2 nRC PAM active spacers and varying MG102 guide scaffolds. Nuclease activity was tested by nucleofecting MG102 mRNAs (500 ng) with sgRNA (450 pmol) targeting individual sites in the AAVS1 locus into K562 cells. Guides scaffolds were from either MG102-2, MG102-39, or MG102-53 guides and spacers had been previously validated with MG102-2 nuclease. 24 AAVS1 guides were tested. After 72 hours, cells were harvested and NGS libraries were prepared to assess editing efficiency. Each bar represents editing efficiency at the site targeted by the specific spacer (x axis).

FIG. 27 depicts ASR MG102-71 mammalian activity with MG102-39 nRC PAM guides. Nuclease activity was tested by nucleofecting MG102-71 mRNA (500 ng) with sgRNA (450 pmol) targeting individual sites in the AAVS1 locus into K562 cells, using the MG102-39 guide scaffold at nRC PAM sites. 96 AAVS1 guides were tested. After 72 hours, cells were harvested and NGS libraries were prepared to assess editing efficiency. Each bar represents editing efficiency at the site targeted by the specific spacer (x axis).

### BRIEF DESCRIPTION OF THE SEQUENCE LISTING

The Sequence Listing filed herewith provides exemplary polynucleotide and polypeptide sequences for use in methods, compositions and systems according to the disclosure. Below are exemplary descriptions of sequences therein.

### MG33 Nucleases

SEQ ID NOs: 1, 463-486, 981-988, and 1289-1312 show the full-length peptide sequences of MG33 nucleases.

SEQ ID NOs: 199 and 669-670 show the nucleotide sequence of a tracrRNA predicted to function with an MG33 nuclease.

SEQ ID NOs: 201 and 1003-1005 show the nucleotide sequences of predicted single-guide RNA (sgRNA) sequences predicted to function with an MG33 nuclease. “N”s denote variable residues and non-N-residues represent the scaffold sequence.

SEQ ID NOs: 1023-1028 show PAM sequences compatible with MG33 nucleases.

SEQ ID NOs: 1045-1054 show CRISPR repeats of MG33 nucleases described herein.

### MG34 Nucleases

SEQ ID NOs: 2-24, 487-488, 1313-1321, 1347, 1350-1368, and 1415-1440 show the full-length peptide sequences of MG34 nucleases.

SEQ ID NOs: 200 and 1348 show the nucleotide sequence of a tracrRNA predicted to function with an MG34 nuclease.

SEQ ID NOs: 202, 203, 613-616, and 1369 show the nucleotide sequences of predicted single-guide RNA (sgRNA) sequences predicted to function with an MG34 nuclease. “N”s denote variable residues and non-N-residues represent the scaffold sequence.

SEQ ID NOs: 1023-1028 and 1441-1450 show PAM sequences compatible with MG34 nucleases.

SEQ ID NOs: 1055-1057, and 1349 show CRISPR repeats of MG34 nucleases described herein.

SEQ ID NOs: 1392-1414 show the nucleotide sequences of MG34 single guide RNAs.

SEQ ID NOs: 1470-2242 show the nucleotide sequences of MG34 chemically synthesized/modified sgRNAs. SEQ ID NOs: 1470-1485 show the nucleotide sequences of MG34-35 sgRNAs targeting AAVS1. SEQ ID NOs: 1486-1489 show the nucleotide sequences of MG34-25 sgRNAs targeting AAVS1. SEQ ID NO: 1490-1493 shows show the nucleotide sequences of MG34-1 sgRNAs targeting AAVS1. SEQ ID NOs: 1494-1685 show the nucleotide sequences of MG34-1 sgRNAs targeting B2M. SEQ ID NOs: 1686-1757 show the nucleotide sequences of MG34-1 sgRNAs targeting hRosa26. SEQ ID NOs: 1758-1806 show the nucleotide sequences of MG34-1 sgRNAs targeting TRAC. SEQ ID NO: 1807 shows the nucleotide sequence of MG34-1 sgRNA targeting VISTA enhancer hs267 regulatory pPE633. SEQ ID NO: 1808 shows the nucleotide sequence of MG34-1 sgRNA targeting Sharpr-MPRA regulatory region 15312 pPE634. SEQ ID NO: 1809 shows the nucleotide sequence of MG34-1 sgRNA targeting EMX1 Intron pPE641. SEQ ID NO: 1810 shows the nucleotide sequence of MG34-1 sgRNA targeting HSPA12A gene (Hsp70 member 12A) pPE635. SEQ ID NOs: 1811-1906 shows the nucleotide sequences of MG34-1 sgRNAs targeting AAVS1. SEQ ID NOs: 1907-2002 shows the nucleotide sequence of MG34-25 sgRNAs targeting AAVS1. SEQ ID NOs: 2003-2098 show the nucleotide sequences of MG102-39 sgRNAs targeting AAVS1. SEQ ID NOs: 2099-2194 show the nucleotide sequences of MG102-53 sgRNAs targeting AAVS1. SEQ ID NOs: 2195-2202 show the nucleotide sequences of MG102-2 sgRNAs targeting AAVS1. SEQ ID NOs: 2203-2210 show the nucleotide sequences of MG102-39 sgRNAs targeting AAVS1. SEQ ID NOs: 2211-2218 show the nucleotide sequences of MG102-53 sgRNAs targeting AAVS1. SEQ ID NOs: 2219-2226 show the nucleotide sequences of MG102-2 sgRNAs targeting TRAC. SEQ ID NOs: 2227-2234 show the nucleotide sequences of MG102-39 sgRNAs targeting TRAC. SEQ ID NOs: 2235-2242 show the nucleotide sequences of MG102-53 sgRNAs targeting TRAC.

### MG35 Nucleases

SEQ ID NOs: 25-198, 221-459, 489-580, 617-668, and 674-675 show the full-length peptide sequences of MG35 nucleases.

SEQ ID NOs: 460-461 show the nucleotide sequences of MG35 tracrRNAs derived from the same loci as MG35 nucleases.

SEQ ID NOs: 462, 676, and 1229-1230 show CRISPR repeats of MG35 nucleases described herein.

SEQ ID NOs: 677-686, 1006-1012, and 1231-1259 show the nucleotide sequences of MG35 single guide RNAs.

SEQ ID NOs: 687-974 show the nucleotide sequences of MG35 single guide RNA encoding sequences.

SEQ ID NOs: 1029-1034 show PAM sequences compatible with MG35 nucleases.

SEQ ID NOs: 1172-1228 show the nucleotide sequences of loci encoding MG35 nucleases described herein.

### MG102 Nucleases

SEQ ID NOs: 581-612, 989-1002, 1260-1273, 1322-1324, and 1329-1346 show the full-length peptide sequences of MG102 nucleases.

SEQ ID NOs: 672-673, 1327-1328, and 1370-1372 show the nucleotide sequences of MG102 tracrRNAs derived from the same loci as MG102 nucleases.

SEQ ID NOs: 205-220 show the sequences of example nuclear localization sequences (NLSs) that, in some embodiments, are appended to nucleases according to the disclosure.

SEQ ID NOs: 1013-1022 and 1376-1391 show the nucleotide sequences of MG102 single guide RNAs.

SEQ ID NOs: 1035-1044 and 1451-1469 show PAM sequences compatible with MG102 nucleases.

SEQ ID NOs: 1058-1072, 1325-1326, and 1373-1375 show CRISPR repeats of MG102 nucleases described herein.

SEQ ID NO: 1171 shows the nucleotide sequence of a locus encoding an MG102 nuclease described herein.

### MG143 Nucleases

SEQ ID NO: 975 shows the full-length peptide sequence of an MG143 nuclease.

SEQ ID NOs: 1073 shows a CRISPR repeat of an MG143 nuclease described herein.

### MG144 Nucleases

SEQ ID NOs: 976-979 and 1274-1288 show the full-length peptide sequences of MG144 nucleases.

SEQ ID NOs: 1074-1077 show CRISPR repeats of MG144 nucleases described herein.

### MG145 Nucleases

SEQ ID NO: 980 shows the full-length peptide sequence of an MG145 nuclease.

SEQ ID NOs: 1078 shows a CRISPR repeat of an MG145 nuclease described herein.

### MG102 TRAC Targeting

SEQ ID NOs: 1079-1082 and 1145-1166 show the DNA sequences of TRAC target sites.

SEQ ID NOs: 1083-1086 and 1123-1144 show the nucleotide sequences of sgRNAs engineered to function with an MG102 nuclease in order to target TRAC.

### MG33 TRAC Targeting

SEQ ID NOs: 1167-1168 show the nucleotide sequences of sgRNAs engineered to function with an MG33 nuclease in order to target TRAC.

SEQ ID NOs: 1169-1170 show the DNA sequences of TRAC target sites.

### AAVS1 Targeting

SEQ ID NOs: 1087-1104 show the nucleotide sequences of sgRNAs engineered to function with an MG102 nuclease in order to target AAVS1.

SEQ ID NOs: 1105-1122 show the DNA sequences of AAVS1 target sites.

### Other

SEQ ID NOs: 2270-2330 show the nucleotide sequences of target sites in the genome.

### DETAILED DESCRIPTION

### Definitions

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

The term “nucleotide,” as used herein, refers to a base-sugar-phosphate combination. Contemplated nucleotides include naturally occurring nucleotides and synthetic nucleotides. Nucleotides are monomeric units of a nucleic acid sequence (e.g., deoxyribonucleic acid (DNA) and ribonucleic acid (RNA)). The term nucleotide includes ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein encompasses dideoxyribonucleoside triphosphates (ddNTPs) and their derivatives. Illustrative examples of ddNTPs include, but are not limited to, ddATP, ddCTP, ddGTP, ddITP, and ddTTP. A nucleotide may be unlabeled or detectably labeled, such as using moieties comprising optically detectable moieties (e.g., fluorophores) or quantum dots. Detectable labels include, for example, radioactive isotopes, fluorescent labels, chemiluminescent labels, bioluminescent labels, and enzyme labels. Fluorescent labels of nucleotides include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides include [R6G]dUTP, [TAMRA]dUTP, [RI10]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, IL; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. The term nucleotide encompasses chemically modified nucleotides. An exemplary chemically-modified nucleotide is biotin-dNTP. Non-limiting examples of biotinylated dNTPs include, biotin-dATP (e.g, bio-N6-ddATP, biotin-14-dATP), biotin-dCTP (e.g, biotin-11-dCTP, biotin-14-dCTP), and biotin-dUTP (e.g, biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. Contemplated polynucleotides include a gene or fragment thereof. Exemplary polynucleotides include, but are not limited to, DNA, RNA, coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, cell-free polynucleotides including cell-free DNA (cfDNA) and cell-free RNA (cfRNA), nucleic acid probes, and primers. In a polynucleotide when referring to a T, a T means U (Uracil) in RNA and T (Thymine) in DNA. A polynucleotide can be exogenous or endogenous to a cell and/or exist in a cell-free environment. The term polynucleotide encompasses modified polynucleotides (e.g., altered backbone, sugar, or nucleobase). If present, modifications to the nucleotide structure are imparted before or after assembly of the polymer. Non-limiting examples of modifications include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. The sequence of nucleotides may be interrupted by non-nucleotide components.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer is interrupted by non-amino acids. The terms include amino acid chains of any length, including full length proteins, and proteins with or without secondary or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids. Modified amino acids include amino acids that have been chemically modified to include a group or a chemical moiety not naturally present on the amino acid. The term “amino acid” includes both D-amino acids and L-amino acids.

As used herein, the “non-native” refers to a nucleic acid or polypeptide sequence that is non-naturally occurring. Non-native refers to a non-naturally occurring nucleic acid or polypeptide sequence that comprises modifications such as mutations, insertions, or deletions. The term non-native encompasses fusion nucleic acids or polypeptides that encodes or exhibits an activity (e.g., enzymatic activity, methyltransferase activity, acetyltransferase activity, kinase activity, ubiquitinating activity, etc.) of the nucleic acid or polypeptide sequence to which the non-native sequence is fused. A non-native nucleic acid or polypeptide sequence includes those linked to a naturally-occurring nucleic acid or polypeptide sequence (or a variant thereof) by genetic engineering to generate a chimeric nucleic acid or polypeptide sequence encoding a chimeric nucleic acid or polypeptide.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type as operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “functional fragment” of a DNA or protein sequence refers to a fragment that retains a biological activity (either functional or structural) that is substantially similar to a biological activity of the full-length DNA or protein sequence. A biological activity of a DNA sequence includes its ability to influence expression in a manner attributed to the full-length sequence.

The terms “engineered,” “synthetic,” and “artificial” are used interchangeably herein to refer to an object that has been modified by human intervention. For example, the terms refer to a polynucleotide or polypeptide that is non-naturally occurring. An engineered peptide has, but does not require, low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains. Non-limiting examples include the following: a nucleic acid modified by changing its sequence to a sequence that does not occur in nature; a nucleic acid modified by ligating it to a nucleic acid that it does not associate with in nature such that the ligated product possesses a function not present in the original nucleic acid; an engineered nucleic acid synthesized in vitro with a sequence that does not exist in nature; a protein modified by changing its amino acid sequence to a sequence that does not exist in nature; an engineered protein acquiring a new function or property. An “engineered” system comprises at least one engineered component.

As used herein, the term “optimally aligned” refers to an alignment of two amino acid sequences that give the highest percent identity score or maximizes the number of matched residues.

The term “tracrRNA” or “tracr sequence” means trans-activating CRISPR RNA. tracrRNA interacts with the CRISPR (cr) RNA to form guide (g) RNA in type II and subtype V-B CRISPR-Cas systems. If the tracrRNA is engineered, it may have about 5%, 10%, 20%, 30%, 40%, 50%, 60%, 70%, 80%, 90%, 95%, or 100% sequence identity and/or sequence similarity to a wild type exemplary tracrRNA sequence (e.g., a tracrRNA from S. pyogenes, S. aureus). tracrRNA may refer to a modified form of a tracrRNA that can comprise a nucleotide change such as a deletion, insertion, or substitution, variant, mutation, or chimera. The term tracrRNA encompasses a nucleic acid that can be at least about 60% identical to a wild type exemplary tracrRNA (e.g., a tracrRNA from S. pyogenes, S. aureus, etc) sequence over a stretch of at least 6 contiguous nucleotides. For example, a tracrRNA sequence has at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes, S. aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” or “guide polynucleotide” refers to a nucleic acid that may hybridize to a target nucleic acid and thereby directs an associated nuclease to the target nucleic acid. A guide nucleic acid is, but is not limited to, RNA (guide RNA or gRNA), DNA, or a mixture of RNA and DNA. A guide nucleic acid can include a crRNA or a tracrRNA or a combination of both. The term guide nucleic acid encompasses an engineered guide nucleic acid and a programmable guide nucleic acid to specifically bind to the target nucleic acid. A portion of the target nucleic acid may be complementary to a portion of the guide nucleic acid. The strand of a double-stranded target polynucleotide that is complementary to and hybridizes with the guide nucleic acid is the complementary strand. The strand of the double-stranded target polynucleotide that is complementary to the complementary strand, and therefore is not complementary to the guide nucleic acid is called noncomplementary strand. A guide nucleic acid having a polynucleotide chain is a “single guide nucleic acid.” A guide nucleic acid having two polynucleotide chains is a “double guide nucleic acid.” If not otherwise specified, the term “guide nucleic acid” is inclusive, referring to both single guide nucleic acids and double guide nucleic acids. A guide nucleic acid may comprise a segment referred to as a “nucleic acid-targeting segment” or a “nucleic acid-targeting sequence,” or a “spacer.” A nucleic acid-targeting segment can include a sub-segment referred to as a “protein binding segment” or “protein binding sequence” or “Cas protein binding segment.”

The term “sequence identity” or “percent identity” in the context of two or more nucleic acids or polypeptide sequences, refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); or CLUSTALW with parameters of the Smith-Waterman homology search algorithm with parameters of a match of 2, a mismatch of −1, and a gap of −1; MUSCLE with default parameters; MAFFT with parameters retree of 2 and maxiterations of 1000; Novafold with default parameters; HMMER hmmalign with default parameters.

As used herein, the term “RuvC_III domain” refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to documented domain sequences, structural alignment to proteins with annotated domains, or by comparison to Hidden Markov Models (HMMs) built based on documented domain sequences (e.g., Pfam HMM PF18541 for RuvC_III).

As used herein, the term “HNH domain” refers to an endonuclease domain having characteristic histidine and asparagine residues. An HNH domain can generally be identified by alignment to documented domain sequences, structural alignment to proteins with annotated domains, or by comparison to Hidden Markov Models (HMMs) built based on documented domain sequences (e.g., Pfam HMM PF01844 for domain HNH).

As used herein, the term “bridge helix domain” or “BH domain” refers to an arginine-rich helix domain present in Cas enzymes that plays an important role in initiating cleavage activity upon binding of target DNA.

As used herein, the term “recognition domain” or “REC domain” refers to a domain thought to interact with the repeat:anti-repeat duplex of the gRNA and to mediate the formation of a Cas endonuclease/gRNA complex.

As used herein, the term “Wedge” (WED) domain refers to a domain (e.g., present in a Cas protein) interacting primarily with repeat:anti-repeat duplex of the sgRNA and PAM duplex. A WED domain can generally be identified by alignment to documented domain sequences, structural alignment to proteins with annotated domains, or by comparison to Hidden Markov Models (HMMs) built based on documented domain sequences.

As used herein, the term “PAM interacting domain” or “PI domain” refers to a domain found in Cas enzymes positioned in the endonuclease-DNA-complex to recognize the PAM sequence on the non-complementary DNA strand of the guide RNA.

As used herein, the term “complex” refers to a joining of at least two components. The two components may each retain the properties/activities they had prior to forming the complex or gain properties as a result of forming the complex. The joining includes, but is not limited to, covalent bonding, non-covalent bonding (i.e., hydrogen bonding, ionic interactions, Van der Waals interactions, and hydrophobic bond), use of a linker, fusion, or any other suitable method. Contemplated components of the complex include polynucleotides, polypeptides, or combinations thereof. For example, a complex comprises an endonuclease and a guide polynucleotide.

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, or T
D=A, G, or T
H=A, C, or T
V=A, C, or G.

### Overview

The discovery of new Cas enzymes with unique functionality and structure offers the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species offers the potential to drastically increase the number of new CRISPR/Cas systems documented and speed the discovery of new oligonucleotide editing functionalities. A recent example of the fruitfulness of such an approach is demonstrated by the 2016 discovery of CasX/CasY CRISPR systems from metagenomic analysis of natural microbial communities.

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity.

Class 1 CRISPR-Cas systems have large, multi-subunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that comprises a highly reduced large subunit nuclease (csf1), two genes for RAMP proteins of the Cas5 (csf3) and Cas7 (csf2) groups, and, in some cases, a gene for a predicted small subunit; such systems are commonly found on endogenous plasmids.

Class 2 CRISPR-Cas systems have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g., Cas9) and the repeat sequence to form a precursor dsRNA structure, which is cleaved by endogenous RNAse III to generate a mature effector enzyme loaded with both tracrRNA and crRNA. Cas II nucleases are DNA nucleases. Type II effectors generally exhibit a structure comprising a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g., Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, type V systems are capable of using the effector nuclease itself to cleave pre-crRNAs. Like Type-II CRISPR-Cas systems, Type V CRISPR-Cas systems are DNA nucleases.

Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRISPR-Cas systems have RNA-guided RNA endonucleases. Instead of RuvC-like domains, the single polypeptide effector of Type VI systems (e.g., Cas13) comprises two HEPN ribonuclease domains. Differing from both Type II and V systems, Type VI systems also appear to not need a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, class 2 CRISPR-Cas have been most widely adopted for engineering and development as designer nuclease/genome editing applications.

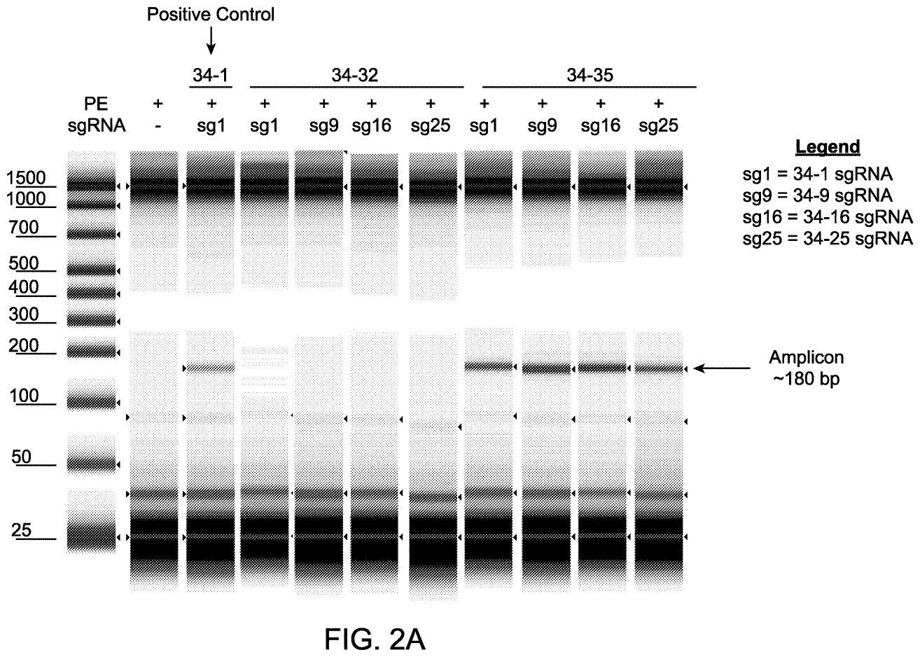

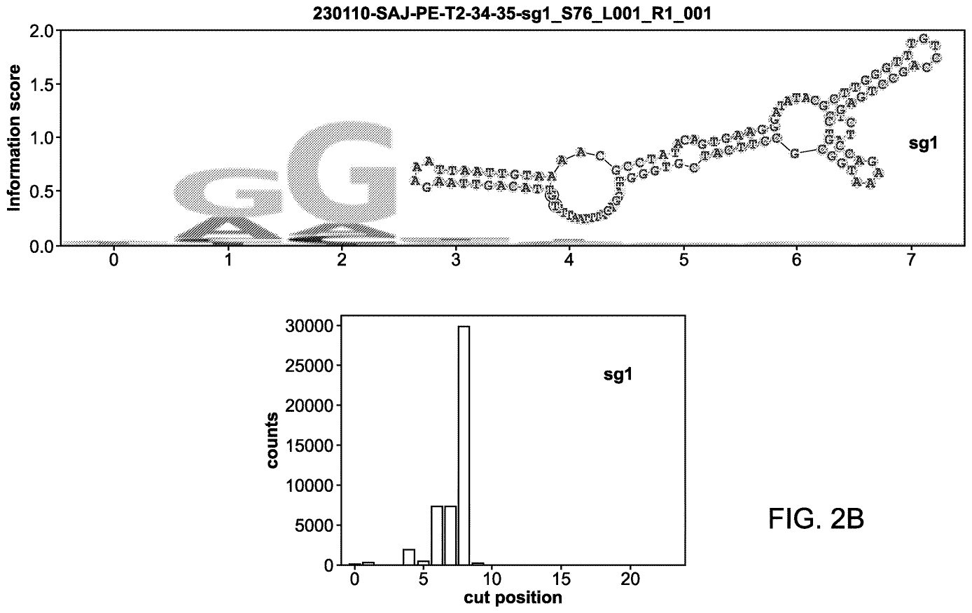

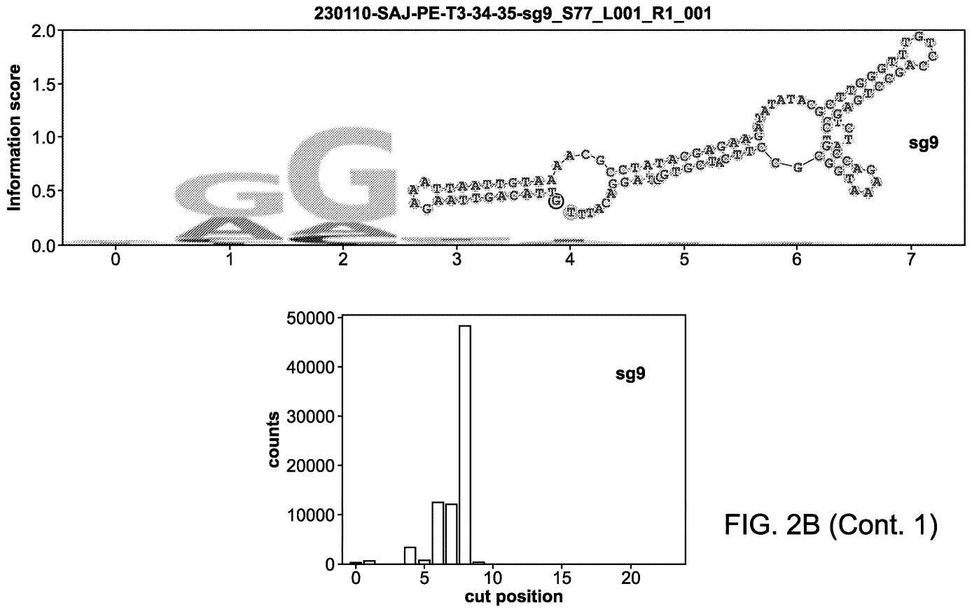

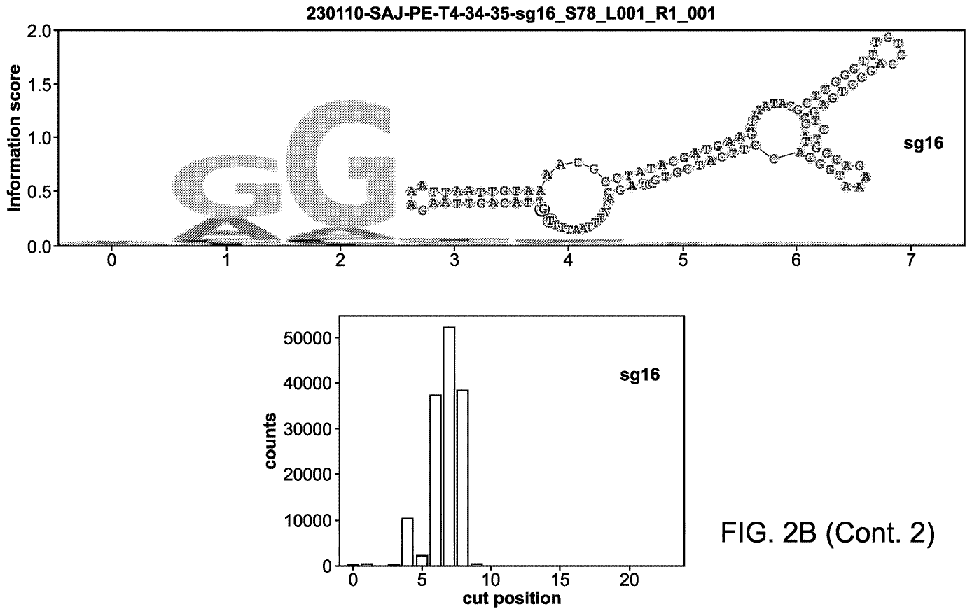

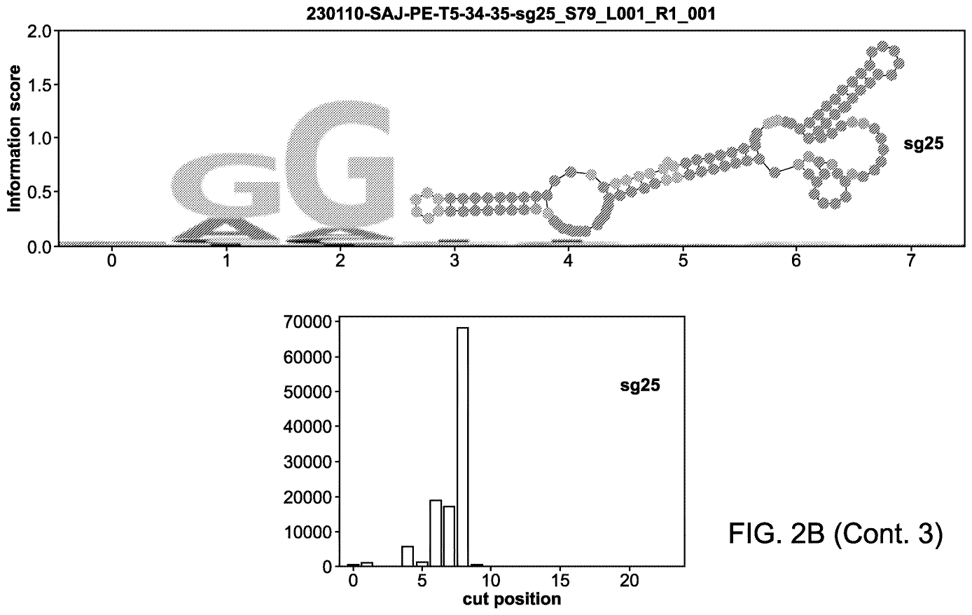

One of the early adaptations of such a system for in vitro use involved (i) recombinantly-expressed, purified full-length Cas9 (e.g., a class 2, Type II Cas enzyme) isolated from S. pyogenes SF370, (ii) purified mature ~42 nt crRNA bearing a ~20 nt 5′ sequence complementary to the target DNA sequence to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg²⁺. A later improved, engineered system involved the crRNA of (ii) joined to the 5′ end of (iii) by a linker (e.g., GAAA) to form a single fused synthetic guide RNA (sgRNA) capable of directing Cas9 to a target by itself (compare top and bottom panel of FIG. 2).

Such engineered systems can be adapted for use in mammalian cells by providing DNA vectors encoding (i) an ORF encoding codon-optimized Cas9 (e.g., a class 2, Type II Cas enzyme) under a suitable mammalian promoter with a C-terminal nuclear localization sequence (e.g., SV40 NLS) and a suitable polyadenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter).

### MG Enzymes

Provided herein, in some embodiments, are engineered nuclease systems comprising a small endonuclease.

In some embodiments, the endonuclease comprises a RuvC-1 domain or a RuvC domain. In some embodiments, the endonuclease comprises an HNH domain. In some embodiments, the endonuclease comprises a RuvC domain and an HNH domain. In some embodiments, the endonuclease comprises an arginine rich region comprising an RRxRR motif or a domain with PF14239 homology. In some embodiments, the endonuclease comprises a REC domain. In some embodiments, the endonuclease comprises a BH (Bridge Helix) domain. In some embodiments, the endonuclease comprises a WED (wedge) domain. In some embodiments, the endonuclease comprises a PI (PAM interacting) domain. In some embodiments, the endonuclease is configured to be selective for a target adjacent motif (TAM) sequence comprising any one of ANGG (SEQ ID NO: 1029), NARAA (SEQ ID NO: 1030), ATGAAA (SEQ ID NO: 1031), ATGA (SEQ ID NO: 1032), or WTGG (SEQ ID NO: 1033).

In some embodiments, the endonuclease is from an uncultivated microorganism. In some embodiments, the endonuclease is a Cas endonuclease. In some embodiments, the endonuclease is a class 2 endonuclease. In some embodiments, the endonuclease is a class 2, type II Cas endonuclease.

In some embodiments, the endonuclease has a molecular weight of about 120 kDa or less, about 110 kDa or less, about 100 kDa or less, about 90 kDa or less, about 80 kDa or less, about 70 kDa or less, about 60 kDa or less, about 50 kDa or less, about 40 kDa or less, about 30 kDa or less, about 20 kDa or less, or about 10 kDa or less. In some embodiments, the endonuclease has a molecular weight of about 120 kDa or less. In some embodiments, the endonuclease has a molecular weight of about 110 kDa or less. In some embodiments, the endonuclease has a molecular weight of about 100 kDa or less. In some embodiments, the endonuclease has a molecular weight of about 90 kDa or less. In some embodiments, the endonuclease has a molecular weight of about 80 kDa or less. In some embodiments, the endonuclease has a molecular weight of about 70 kDa or less. In some embodiments, the endonuclease has a molecular weight of about 60 kDa or less. In some embodiments, the endonuclease has a molecular weight of about 50 kDa or less. In some embodiments, the endonuclease has a molecular weight of about 40 kDa or less. In some embodiments, the endonuclease has a molecular weight of about 30 kDa or less. In some embodiments, the endonuclease has a molecular weight of about 20 kDa or less. In some embodiments, the endonuclease has a molecular weight of about 10 kDa or less.

In some embodiments, the endonuclease is not a Cas9 endonuclease, a Cas14 endonuclease, a Cas12a endonuclease, a Cas12b endonuclease, a Cas 12c endonuclease, a Cas12d endonuclease, a Cas12e endonuclease, a Cas13a endonuclease, a Cas13b endonuclease, a Cas13c endonuclease, or a Cas 13d endonuclease.

In some embodiments, the endonuclease has less than less than 80% identity, less than 75% identity, less than 70% identity, less than 65% identity, less than 60% identity, less than 55% identity, or less than 50% identity to a Cas9 endonuclease. In some embodiments, the endonuclease has less than less than 80% identity to a Cas9 endonuclease. In some embodiments, the endonuclease has less than less than 75% identity to a Cas9 endonuclease. In some embodiments, the endonuclease has less than less than 70% identity to a Cas9 endonuclease. In some embodiments, the endonuclease has less than less than 65% identity to a Cas9 endonuclease. In some embodiments, the endonuclease has less than less than 60% identity to a Cas9 endonuclease. In some embodiments, the endonuclease has less than less than 55% identity to a Cas9 endonuclease. In some embodiments, the endonuclease has less than less than 50% identity to a Cas9 endonuclease.

In some embodiments, the endonuclease comprises a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 70% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 75% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 75% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 80% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 81% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 82% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 83% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 84% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 85% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 86% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 87% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 88% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 89% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 90% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 91% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 92% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 93% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 94% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 95% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 96% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 97% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 98% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 99% sequence identity to any one of SEQ ID NOs: 1-198, 221-459, 463-612, 617-668, 674-675, 975-1002, 1322-1324, 1329-1347, 1350-1368, and 1415-1440. In some embodiments, the endonuclease comprises a sequence with at least 100% sequence identity to any one of SEQ ID NOs: 1-198, 221-459, 463-612, 617-668, 674-675, 975-1002, 1322-1324, 1329-1347, 1350-1368, and 1415-1440.

In some embodiments, the endonuclease comprises a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 1322-1324, 1329-1347, 1350-1368, and 1415-1440. In some embodiments, the endonuclease comprises a sequence with at least 70% sequence identity to any one of SEQ ID NOs: 1322-1324, 1329-1347, 1350-1368, and 1415-1440. In some embodiments, the endonuclease comprises a sequence with at least 75% sequence identity to any one of SEQ ID NOs: 1322-1324, 1329-1347, 1350-1368, and 1415-1440. In some embodiments, the endonuclease comprises a sequence with at least 75% sequence identity to any one of SEQ ID NOs: 1322-1324, 1329-1347, 1350-1368, and 1415-1440. In some embodiments, the endonuclease comprises a sequence with at least 80% sequence identity to any one of SEQ ID NOs: 1322-1324, 1329-1347, 1350-1368, and 1415-1440. In some embodiments, the endonuclease comprises a sequence with at least 81% sequence identity to any one of SEQ ID NOs: 1322-1324, 1329-1347, 1350-1368, and 1415-1440. In some embodiments, the endonuclease comprises a sequence with at least 82% sequence identity to any one of SEQ ID NOs: 1322-1324, 1329-1347, 1350-1368, and 1415-1440. In some embodiments, the endonuclease comprises a sequence with at least 83% sequence identity to any one of SEQ ID NOs: 1322-1324, 1329-1347, 1350-1368, and 1415-1440. In some embodiments, the endonuclease comprises a sequence with at least 84% sequence identity to any one of SEQ ID NOs: 1322-1324, 1329-1347, 1350-1368, and 1415-1440. In some embodiments, the endonuclease comprises a sequence with at least 85% sequence identity to any one of SEQ ID NOs: 1322-1324, 1329-1347, 1350-1368, and 1415-1440. In some embodiments, the endonuclease comprises a sequence with at least 86% sequence identity to any one of SEQ ID NOs: 1322-1324, 1329-1347, 1350-1368, and 1415-1440. In some embodiments, the endonuclease comprises a sequence with at least 87% sequence identity to any one of SEQ ID NOs: 1322-1324, 1329-1347, 1350-1368, and 1415-1440. In some embodiments, the endonuclease comprises a sequence with at least 88% sequence identity to any one of SEQ ID NOs: 1322-1324, 1329-1347, 1350-1368, and 1415-1440. In some embodiments, the endonuclease comprises a sequence with at least 89% sequence identity to any one of SEQ ID NOs: 1322-1324, 1329-1347, 1350-1368, and 1415-1440. In some embodiments, the endonuclease comprises a sequence with at least 90% sequence identity to any one of SEQ ID NOs: 1322-1324, 1329-1347, 1350-1368, and 1415-1440. In some embodiments, the endonuclease comprises a sequence with at least 91% sequence identity to any one of SEQ ID NOs: 1322-1324, 1329-1347, 1350-1368, and 1415-1440. In some embodiments, the endonuclease comprises a sequence with at least 92% sequence identity to any one of SEQ ID NOs: 1322-1324, 1329-1347, 1350-1368, and 1415-1440. In some embodiments, the endonuclease comprises a sequence with at least 93% sequence identity to any one of SEQ ID NOs: 1322-1324, 1329-1347, 1350-1368, and 1415-1440. In some embodiments, the endonuclease comprises a sequence with at least 94% sequence identity to any one of SEQ ID NOs: 1322-1324, 1329-1347, 1350-1368, and 1415-1440. In some embodiments, the endonuclease comprises a sequence with at least 95% sequence identity to any one of SEQ ID NOs: 1322-1324, 1329-1347, 1350-1368, and 1415-1440. In some embodiments, the endonuclease comprises a sequence with at least 96% sequence identity to any one of SEQ ID NOs: 1322-1324, 1329-1347, 1350-1368, and 1415-1440. In some embodiments, the endonuclease comprises a sequence with at least 97% sequence identity to any one of SEQ ID NOs: 1322-1324, 1329-1347, 1350-1368, and 1415-1440. In some embodiments, the endonuclease comprises a sequence with at least 98% sequence identity to any one of SEQ ID NOs: 1322-1324, 1329-1347, 1350-1368, and 1415-1440. In some embodiments, the endonuclease comprises a sequence with at least 99% sequence identity to any one of SEQ ID NOs: 1322-1324, 1329-1347, 1350-1368, and 1415-1440. In some embodiments, the endonuclease comprises a sequence with at least 100% sequence identity to any one of SEQ ID NOs: 1322-1324, 1329-1347, 1350-1368, and 1415-1440.

In some embodiments, the endonuclease is a MG33 nuclease. In some embodiments, the endonuclease comprises a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 1, 463-486, 981-988, and 1289-1312. In some embodiments, the endonuclease comprises a sequence with at least 70% sequence identity to any one of SEQ ID NOs: 1, 463-486, 981-988, and 1289-1312. In some embodiments, the endonuclease comprises a sequence with at least 75% sequence identity to any one of SEQ ID NOs: 1, 463-486, 981-988, and 1289-1312. In some embodiments, the endonuclease comprises a sequence with at least 75% sequence identity to any one of SEQ ID NOs: 1, 463-486, 981-988, and 1289-1312. In some embodiments, the endonuclease comprises a sequence with at least 80% sequence identity to any one of SEQ ID NOs: 1, 463-486, 981-988, and 1289-1312. In some embodiments, the endonuclease comprises a sequence with at least 81% sequence identity to any one of SEQ ID NOs: 1, 463-486, 981-988, and 1289-1312. In some embodiments, the endonuclease comprises a sequence with at least 82% sequence identity to any one of SEQ ID NOs: 1, 463-486, 981-988, and 1289-1312. In some embodiments, the endonuclease comprises a sequence with at least 83% sequence identity to any one of SEQ ID NOs: 1, 463-486, 981-988, and 1289-1312. In some embodiments, the endonuclease comprises a sequence with at least 84% sequence identity to any one of SEQ ID NOs: 1, 463-486, 981-988, and 1289-1312. In some embodiments, the endonuclease comprises a sequence with at least 85% sequence identity to any one of SEQ ID NOs: 1, 463-486, 981-988, and 1289-1312. In some embodiments, the endonuclease comprises a sequence with at least 86% sequence identity to any one of SEQ ID NOs: 1, 463-486, 981-988, and 1289-1312. In some embodiments, the endonuclease comprises a sequence with at least 87% sequence identity to any one of SEQ ID NOs: 1, 463-486, 981-988, and 1289-1312. In some embodiments, the endonuclease comprises a sequence with at least 88% sequence identity to any one of SEQ ID NOs: 1, 463-486, 981-988, and 1289-1312. In some embodiments, the endonuclease comprises a sequence with at least 89% sequence identity to any one of SEQ ID NOs: 1, 463-486, 981-988, and 1289-1312. In some embodiments, the endonuclease comprises a sequence with at least 90% sequence identity to any one of SEQ ID NOs: 1, 463-486, 981-988, and 1289-1312. In some embodiments, the endonuclease comprises a sequence with at least 91% sequence identity to any one of SEQ ID NOs: 1, 463-486, 981-988, and 1289-1312. In some embodiments, the endonuclease comprises a sequence with at least 92% sequence identity to any one of SEQ ID NOs: 1, 463-486, 981-988, and 1289-1312. In some embodiments, the endonuclease comprises a sequence with at least 93% sequence identity to any one of SEQ ID NOs: 1, 463-486, 981-988, and 1289-1312. In some embodiments, the endonuclease comprises a sequence with at least 94% sequence identity to any one of SEQ ID NOs: 1, 463-486, 981-988, and 1289-1312. In some embodiments, the endonuclease comprises a sequence with at least 95% sequence identity to any one of SEQ ID NOs: 1, 463-486, 981-988, and 1289-1312. In some embodiments, the endonuclease comprises a sequence with at least 96% sequence identity to any one of SEQ ID NOs: 1, 463-486, 981-988, and 1289-1312. In some embodiments, the endonuclease comprises a sequence with at least 97% sequence identity to any one of SEQ ID NOs: 1, 463-486, 981-988, and 1289-1312. In some embodiments, the endonuclease comprises a sequence with at least 98% sequence identity to any one of SEQ ID NOs: 1, 463-486, 981-988, and 1289-1312. In some embodiments, the endonuclease comprises a sequence with at least 99% sequence identity to any one of SEQ ID NOs: 1, 463-486, 981-988, and 1289-1312. In some embodiments, the endonuclease comprises a sequence with at least 100% sequence identity to any one of SEQ ID NOs: 1, 463-486, 981-988, and 1289-1312.

In some embodiments, the nuclease is a MG34 nuclease. In some embodiments, the endonuclease comprises a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 2-24, 487-488, 1313-1321, 1347, 1350-1368, and 1415-1440. In some embodiments, the endonuclease comprises a sequence with at least 70% sequence identity to any one of SEQ ID NOs: 2-24, 487-488, 1313-1321, 1347, 1350-1368, and 1415-1440. In some embodiments, the endonuclease comprises a sequence with at least 75% sequence identity to any one of SEQ ID NOs: 2-24, 487-488, 1313-1321, 1347, 1350-1368, and 1415-1440. In some embodiments, the endonuclease comprises a sequence with at least 75% sequence identity to any one of SEQ ID NOs: 2-24, 487-488, 1313-1321, 1347, 1350-1368, and 1415-1440. In some embodiments, the endonuclease comprises a sequence with at least 80% sequence identity to any one of SEQ ID NOs: 2-24, 487-488, 1313-1321, 1347, 1350-1368, and 1415-1440. In some embodiments, the endonuclease comprises a sequence with at least 81% sequence identity to any one of SEQ ID NOs: 2-24, 487-488, 1313-1321, 1347, 1350-1368, and 1415-1440. In some embodiments, the endonuclease comprises a sequence with at least 82% sequence identity to any one of SEQ ID NOs: 2-24, 487-488, 1313-1321, 1347, 1350-1368, and 1415-1440. In some embodiments, the endonuclease comprises a sequence with at least 83% sequence identity to any one of SEQ ID NOs: 2-24, 487-488, 1313-1321, 1347, 1350-1368, and 1415-1440. In some embodiments, the endonuclease comprises a sequence with at least 84% sequence identity to any one of SEQ ID NOs: 2-24, 487-488, 1313-1321, 1347, 1350-1368, and 1415-1440. In some embodiments, the endonuclease comprises a sequence with at least 85% sequence identity to any one of SEQ ID NOs: 2-24, 487-488, 1313-1321, 1347, 1350-1368, and 1415-1440. In some embodiments, the endonuclease comprises a sequence with at least 86% sequence identity to any one of SEQ ID NOs: 2-24, 487-488, 1313-1321, 1347, 1350-1368, and 1415-1440. In some embodiments, the endonuclease comprises a sequence with at least 87% sequence identity to any one of SEQ ID NOs: 2-24, 487-488, 1313-1321, 1347, 1350-1368, and 1415-1440. In some embodiments, the endonuclease comprises a sequence with at least 88% sequence identity to any one of SEQ ID NOs: 2-24, 487-488, 1313-1321, 1347, 1350-1368, and 1415-1440. In some embodiments, the endonuclease comprises a sequence with at least 89% sequence identity to any one of SEQ ID NOs: 2-24, 487-488, 1313-1321, 1347, 1350-1368, and 1415-1440. In some embodiments, the endonuclease comprises a sequence with at least 90% sequence identity to any one of SEQ ID NOs: 2-24, 487-488, 1313-1321, 1347, 1350-1368, and 1415-1440. In some embodiments, the endonuclease comprises a sequence with at least 91% sequence identity to any one of SEQ ID NOs: 2-24, 487-488, 1313-1321, 1347, 1350-1368, and 1415-1440. In some embodiments, the endonuclease comprises a sequence with at least 92% sequence identity to any one of SEQ ID NOs: 2-24, 487-488, 1313-1321, 1347, 1350-1368, and 1415-1440. In some embodiments, the endonuclease comprises a sequence with at least 93% sequence identity to any one of SEQ ID NOs: 2-24, 487-488, 1313-1321, 1347, 1350-1368, and 1415-1440. In some embodiments, the endonuclease comprises a sequence with at least 94% sequence identity to any one of SEQ ID NOs: 2-24, 487-488, 1313-1321, 1347, 1350-1368, and 1415-1440. In some embodiments, the endonuclease comprises a sequence with at least 95% sequence identity to any one of SEQ ID NOs: 2-24, 487-488, 1313-1321, 1347, 1350-1368, and 1415-1440. In some embodiments, the endonuclease comprises a sequence with at least 96% sequence identity to any one of SEQ ID NOs: 2-24, 487-488, 1313-1321, 1347, 1350-1368, and 1415-1440. In some embodiments, the endonuclease comprises a sequence with at least 97% sequence identity to any one of SEQ ID NOs: 2-24, 487-488, 1313-1321, 1347, 1350-1368, and 1415-1440. In some embodiments, the endonuclease comprises a sequence with at least 98% sequence identity to any one of SEQ ID NOs: 2-24, 487-488, 1313-1321, 1347, 1350-1368, and 1415-1440. In some embodiments, the endonuclease comprises a sequence with at least 99% sequence identity to any one of SEQ ID NOs: 2-24, 487-488, 1313-1321, 1347, 1350-1368, and 1415-1440. In some embodiments, the endonuclease comprises a sequence with at least 100% sequence identity to any one of SEQ ID NOs: 2-24, 487-488, 1313-1321, 1347, 1350-1368, and 1415-1440.

In some embodiments, the endonuclease is a MG35 nuclease. In some embodiments, the endonuclease comprises a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 25-198, 221-459, 489-580, 617-668, and 674-675. In some embodiments, the endonuclease comprises a sequence with at least 70% sequence identity to any one of SEQ ID NOs: 25-198, 221-459, 489-580, 617-668, and 674-675. In some embodiments, the endonuclease comprises a sequence with at least 75% sequence identity to any one of SEQ ID NOs: 25-198, 221-459, 489-580, 617-668, and 674-675. In some embodiments, the endonuclease comprises a sequence with at least 75% sequence identity to any one of SEQ ID NOs: 25-198, 221-459, 489-580, 617-668, and 674-675. In some embodiments, the endonuclease comprises a sequence with at least 80% sequence identity to any one of SEQ ID NOs: 25-198, 221-459, 489-580, 617-668, and 674-675. In some embodiments, the endonuclease comprises a sequence with at least 81% sequence identity to any one of SEQ ID NOs: 25-198, 221-459, 489-580, 617-668, and 674-675. In some embodiments, the endonuclease comprises a sequence with at least 82% sequence identity to any one of SEQ ID NOs: 25-198, 221-459, 489-580, 617-668, and 674-675. In some embodiments, the endonuclease comprises a sequence with at least 83% sequence identity to any one of SEQ ID NOs: 25-198, 221-459, 489-580, 617-668, and 674-675. In some embodiments, the endonuclease comprises a sequence with at least 84% sequence identity to any one of SEQ ID NOs: 25-198, 221-459, 489-580, 617-668, and 674-675. In some embodiments, the endonuclease comprises a sequence with at least 85% sequence identity to any one of SEQ ID NOs: 25-198, 221-459, 489-580, 617-668, and 674-675. In some embodiments, the endonuclease comprises a sequence with at least 86% sequence identity to any one of SEQ ID NOs: 25-198, 221-459, 489-580, 617-668, and 674-675. In some embodiments, the endonuclease comprises a sequence with at least 87% sequence identity to any one of SEQ ID NOs: 25-198, 221-459, 489-580, 617-668, and 674-675. In some embodiments, the endonuclease comprises a sequence with at least 88% sequence identity to any one of SEQ ID NOs: 25-198, 221-459, 489-580, 617-668, and 674-675. In some embodiments, the endonuclease comprises a sequence with at least 89% sequence identity to any one of SEQ ID NOs: 25-198, 221-459, 489-580, 617-668, and 674-675. In some embodiments, the endonuclease comprises a sequence with at least 90% sequence identity to any one of SEQ ID NOs: 25-198, 221-459, 489-580, 617-668, and 674-675. In some embodiments, the endonuclease comprises a sequence with at least 91% sequence identity to any one of SEQ ID NOs: 25-198, 221-459, 489-580, 617-668, and 674-675. In some embodiments, the endonuclease comprises a sequence with at least 92% sequence identity to any one of SEQ ID NOs: 25-198, 221-459, 489-580, 617-668, and 674-675. In some embodiments, the endonuclease comprises a sequence with at least 93% sequence identity to any one of SEQ ID NOs: 25-198, 221-459, 489-580, 617-668, and 674-675. In some embodiments, the endonuclease comprises a sequence with at least 94% sequence identity to any one of SEQ ID NOs: 25-198, 221-459, 489-580, 617-668, and 674-675. In some embodiments, the endonuclease comprises a sequence with at least 95% sequence identity to any one of SEQ ID NOs: 25-198, 221-459, 489-580, 617-668, and 674-675. In some embodiments, the endonuclease comprises a sequence with at least 96% sequence identity to any one of SEQ ID NOs: 25-198, 221-459, 489-580, 617-668, and 674-675. In some embodiments, the endonuclease comprises a sequence with at least 97% sequence identity to any one of SEQ ID NOs: 25-198, 221-459, 489-580, 617-668, and 674-675. In some embodiments, the endonuclease comprises a sequence with at least 98% sequence identity to any one of SEQ ID NOs: 25-198, 221-459, 489-580, 617-668, and 674-675. In some embodiments, the endonuclease comprises a sequence with at least 99% sequence identity to any one of SEQ ID NOs: 25-198, 221-459, 489-580, 617-668, and 674-675. In some embodiments, the endonuclease comprises a sequence with at least 100% sequence identity to any one of SEQ ID NOs: 25-198, 221-459, 489-580, 617-668, and 674-675.

In some embodiments, the endonuclease is a MG102 nuclease. In some embodiments, the endonuclease comprises a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 581-612, 989-1002, 1260-1273, 1322-1324, and 1329-1346. In some embodiments, the endonuclease comprises a sequence with at least 70% sequence identity to any one of SEQ ID NOs: 581-612, 989-1002, 1260-1273, 1322-1324, and 1329-1346. In some embodiments, the endonuclease comprises a sequence with at least 75% sequence identity to any one of SEQ ID NOs: 581-612, 989-1002, 1260-1273, 1322-1324, and 1329-1346. In some embodiments, the endonuclease comprises a sequence with at least 75% sequence identity to any one of SEQ ID NOs: 581-612, 989-1002, 1260-1273, 1322-1324, and 1329-1346. In some embodiments, the endonuclease comprises a sequence with at least 80% sequence identity to any one of SEQ ID NOs: 581-612, 989-1002, 1260-1273, 1322-1324, and 1329-1346. In some embodiments, the endonuclease comprises a sequence with at least 81% sequence identity to any one of SEQ ID NOs: 581-612, 989-1002, 1260-1273, 1322-1324, and 1329-1346. In some embodiments, the endonuclease comprises a sequence with at least 82% sequence identity to any one of SEQ ID NOs: 581-612, 989-1002, 1260-1273, 1322-1324, and 1329-1346. In some embodiments, the endonuclease comprises a sequence with at least 83% sequence identity to any one of SEQ ID NOs: 581-612, 989-1002, 1260-1273, 1322-1324, and 1329-1346. In some embodiments, the endonuclease comprises a sequence with at least 84% sequence identity to any one of SEQ ID NOs: 581-612, 989-1002, 1260-1273, 1322-1324, and 1329-1346. In some embodiments, the endonuclease comprises a sequence with at least 85% sequence identity to any one of SEQ ID NOs: 581-612, 989-1002, 1260-1273, 1322-1324, and 1329-1346. In some embodiments, the endonuclease comprises a sequence with at least 86% sequence identity to any one of SEQ ID NOs: 581-612, 989-1002, 1260-1273, 1322-1324, and 1329-1346. In some embodiments, the endonuclease comprises a sequence with at least 87% sequence identity to any one of SEQ ID NOs: 581-612, 989-1002, 1260-1273, 1322-1324, and 1329-1346. In some embodiments, the endonuclease comprises a sequence with at least 88% sequence identity to any one of SEQ ID NOs: 581-612, 989-1002, 1260-1273, 1322-1324, and 1329-1346. In some embodiments, the endonuclease comprises a sequence with at least 89% sequence identity to any one of SEQ ID NOs: 581-612, 989-1002, 1260-1273, 1322-1324, and 1329-1346. In some embodiments, the endonuclease comprises a sequence with at least 90% sequence identity to any one of SEQ ID NOs: 581-612, 989-1002, 1260-1273, 1322-1324, and 1329-1346. In some embodiments, the endonuclease comprises a sequence with at least 91% sequence identity to any one of SEQ ID NOs: 581-612, 989-1002, 1260-1273, 1322-1324, and 1329-1346. In some embodiments, the endonuclease comprises a sequence with at least 92% sequence identity to any one of SEQ ID NOs: 581-612, 989-1002, 1260-1273, 1322-1324, and 1329-1346. In some embodiments, the endonuclease comprises a sequence with at least 93% sequence identity to any one of SEQ ID NOs: 581-612, 989-1002, 1260-1273, 1322-1324, and 1329-1346. In some embodiments, the endonuclease comprises a sequence with at least 94% sequence identity to any one of SEQ ID NOs: 581-612, 989-1002, 1260-1273, 1322-1324, and 1329-1346. In some embodiments, the endonuclease comprises a sequence with at least 95% sequence identity to any one of SEQ ID NOs: 581-612, 989-1002, 1260-1273, 1322-1324, and 1329-1346. In some embodiments, the endonuclease comprises a sequence with at least 96% sequence identity to any one of SEQ ID NOs: 581-612, 989-1002, 1260-1273, 1322-1324, and 1329-1346. In some embodiments, the endonuclease comprises a sequence with at least 97% sequence identity to any one of SEQ ID NOs: 581-612, 989-1002, 1260-1273, 1322-1324, and 1329-1346. In some embodiments, the endonuclease comprises a sequence with at least 98% sequence identity to any one of SEQ ID NOs: 581-612, 989-1002, 1260-1273, 1322-1324, and 1329-1346. In some embodiments, the endonuclease comprises a sequence with at least 99% sequence identity to any one of SEQ ID NOs: 581-612, 989-1002, 1260-1273, 1322-1324, and 1329-1346. In some embodiments, the endonuclease comprises a sequence with at least 100% sequence identity to any one of SEQ ID NOs: 581-612, 989-1002, 1260-1273, 1322-1324, and 1329-1346.

In some embodiments, the endonuclease is a MG144 nuclease. In some embodiments, the endonuclease comprises a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 976-979 and 1274-1288. In some embodiments, the endonuclease comprises a sequence with at least 70% sequence identity to any one of SEQ ID NOs: 976-979 and 1274-1288. In some embodiments, the endonuclease comprises a sequence with at least 75% sequence identity to any one of SEQ ID NOs: 976-979 and 1274-1288. In some embodiments, the endonuclease comprises a sequence with at least 75% sequence identity to any one of SEQ ID NOs: 976-979 and 1274-1288. In some embodiments, the endonuclease comprises a sequence with at least 80% sequence identity to any one of SEQ ID NOs: 976-979 and 1274-1288. In some embodiments, the endonuclease comprises a sequence with at least 81% sequence identity to any one of SEQ ID NOs: 976-979 and 1274-1288. In some embodiments, the endonuclease comprises a sequence with at least 82% sequence identity to any one of SEQ ID NOs: 976-979 and 1274-1288. In some embodiments, the endonuclease comprises a sequence with at least 83% sequence identity to any one of SEQ ID NOs: 976-979 and 1274-1288. In some embodiments, the endonuclease comprises a sequence with at least 84% sequence identity to any one of SEQ ID NOs: 976-979 and 1274-1288. In some embodiments, the endonuclease comprises a sequence with at least 85% sequence identity to any one of SEQ ID NOs: 976-979 and 1274-1288. In some embodiments, the endonuclease comprises a sequence with at least 86% sequence identity to any one of SEQ ID NOs: 976-979 and 1274-1288. In some embodiments, the endonuclease comprises a sequence with at least 87% sequence identity to any one of SEQ ID NOs: 976-979 and 1274-1288. In some embodiments, the endonuclease comprises a sequence with at least 88% sequence identity to any one of SEQ ID NOs: 976-979 and 1274-1288. In some embodiments, the endonuclease comprises a sequence with at least 89% sequence identity to any one of SEQ ID NOs: 976-979 and 1274-1288. In some embodiments, the endonuclease comprises a sequence with at least 90% sequence identity to any one of SEQ ID NOs: 976-979 and 1274-1288. In some embodiments, the endonuclease comprises a sequence with at least 91% sequence identity to any one of SEQ ID NOs: 976-979 and 1274-1288. In some embodiments, the endonuclease comprises a sequence with at least 92% sequence identity to any one of SEQ ID NOs: 976-979 and 1274-1288. In some embodiments, the endonuclease comprises a sequence with at least 93% sequence identity to any one of SEQ ID NOs: 976-979 and 1274-1288. In some embodiments, the endonuclease comprises a sequence with at least 94% sequence identity to any one of SEQ ID NOs: 976-979 and 1274-1288. In some embodiments, the endonuclease comprises a sequence with at least 95% sequence identity to any one of SEQ ID NOs: 976-979 and 1274-1288. In some embodiments, the endonuclease comprises a sequence with at least 96% sequence identity to any one of SEQ ID NOs: 976-979 and 1274-1288. In some embodiments, the endonuclease comprises a sequence with at least 97% sequence identity to any one of SEQ ID NOs: 976-979 and 1274-1288. In some embodiments, the endonuclease comprises a sequence with at least 98% sequence identity to any one of SEQ ID NOs: 976-979 and 1274-1288. In some embodiments, the endonuclease comprises a sequence with at least 99% sequence identity to any one of SEQ ID NOs: 976-979 and 1274-1288. In some embodiments, the endonuclease comprises a sequence with at least 100% sequence identity to any one of SEQ ID NOs: 976-979 and 1274-1288.

Described herein, in certain embodiments, are engineered nuclease systems comprising: an endonuclease comprising a sequence having at least 80% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 80% identity to any one of SEQ ID NOs: 1350-1368 and 1415-1440. In some embodiments, the endonuclease comprises a sequence having at least 80% identity to any one of SEQ ID NOs: 1324 and 1329-1346. In some embodiments, the endonuclease comprises a sequence with at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 1350-1368 and 1415-1440. In some embodiments, the endonuclease comprises a sequence having at least 80% identity to any one of SEQ ID NOs: 1350-1368 and 1415-1440. In some embodiments, the endonuclease comprises a sequence having at least 80% identity to any one of SEQ ID NOs: 1324 and 1329-1346.

Described herein, in certain embodiments, are engineered nuclease systems comprising: an endonuclease comprising a sequence having at least 90% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence with at least 90%, at least 91%, at least 92%, at least 93%, at least 94%, at least 95%, at least 96%, at least 97%, at least 98%, or at least 99% sequence identity to any one of SEQ ID NOs: SEQ ID NOs: 1324, 1329-1346, 1350-1368, and 1415-1440. In some embodiments, the endonuclease comprises a sequence having at least 90% identity to 1350-1368 and 1415-1440. In some embodiments, the endonuclease comprises a sequence having at least 90% identity to any one of SEQ ID NOs: 1324 and 1329-1346.

Described herein, in certain embodiments, are engineered nuclease systems comprising an endonuclease comprising a sequence having at least 95% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 95% identity to 1350-1368 and 1415-1440. In some embodiments, the endonuclease comprises a sequence having at least 95% identity to any one of SEQ ID NOs: 1324 and 1329-1346.

Described herein, in certain embodiments, are engineered nuclease systems comprising an endonuclease comprising a sequence having at least 99% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 99% identity to 1350-1368 and 1415-1440. In some embodiments, the endonuclease comprises a sequence having at least 99% identity to any one of SEQ ID NOs: 1324 and 1329-1346.

Described herein, in certain embodiments, are engineered nuclease systems comprising an endonuclease comprising a sequence having 100% sequence identity to any one of SEQ ID NOs: 1323-1324, 1329-1347, 1350-1368, and 1415-1440; and an engineered guide polynucleotide that forms a complex with the endonuclease and hybridizes to a target nucleic acid sequence. In some embodiments, the endonuclease comprises a sequence having 100% sequence identity to any one of SEQ ID NOs: 1347, 1350-1368, and 1415-1440. In some embodiments, the endonuclease comprises a sequence having 100% identity to any one of SEQ ID NOs: 1323, 1324 and 1329-1346.

In some embodiments, the present disclosure provides an endonuclease described herein configured to induce a double stranded break proximal to said target locus 5′ to a protospacer adjacent motif (PAM). In some embodiments, the endonuclease induces a double-stranded break 6-8 nucleotides from the PAM or 7 nucleotides from the PAM. In some embodiments, the present disclosure provides an endonuclease described herein configured to induce a single-stranded break proximal to said target locus 5′ to a protospacer adjacent motif (PAM). In some embodiments, the endonuclease induces a single-stranded break 6-8 nucleotides from the PAM or 7 nucleotides from the PAM. In some embodiments, an endonuclease configured to induce a single-stranded break comprises an inactivating mutation in one or more catalytic residues of an endonuclease described herein.

In some embodiments, the endonuclease comprises one or more nuclear localization sequences (NLSs) proximal to an N- or C-terminus of the endonuclease. In some embodiments, the NLS comprises a sequence selected from SEQ ID NOs: 205-220 and 2243-2268. In some embodiments, the NLS comprises a sequence of any one of SEQ ID NOs: 205-220 and 2243-2268, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 205-220 and 2243-2268. In some embodiments, the NLS comprises a sequence having at least about 80% identity to SEQ ID NOs: 205-220 and 2243-2268. In some embodiments, the NLS comprises a sequence having at least about 85% identity to SEQ ID NOs: 205-220 and 2243-2268. In some embodiments, the NLS comprises a sequence having at least about 90% identity to SEQ ID NOs: 205-220 and 2243-2268. In some embodiments, the NLS comprises a sequence having at least about 91% identity to SEQ ID NOs: 205-220 and 2243-2268. In some embodiments, the NLS comprises a sequence having at least about 92% identity to SEQ ID NOs: 205-220 and 2243-2268. In some embodiments, the NLS comprises a sequence having at least about 93E identity to SEQ ID NOs: 205-220 and 2243-2268. In some embodiments, the NLS comprises a sequence having at least about 94% identity to SEQ ID NOs: 205-220 and 2243-2268. In some embodiments, the NLS comprises a sequence having at least about 95% identity to SEQ ID NOs: 205-220 and 2243-2268. In some embodiments, the NLS comprises a sequence having at least about 96% identity to SEQ ID NOs: 205-220 and 2243-2268. In some embodiments, the NLS comprises a sequence having at least about 97K identity to SEQ ID NOs: 205-220 and 2243-2268. In some embodiments, the NLS comprises a sequence having at least about 98% identity to SEQ ID NOs: 205-220 and 2243-2268. In some embodiments, the NLS comprises a sequence having at least about 99% identity to SEQ ID NOs: 205-220 and 2243-2268. In some embodiments, the NLS comprises a sequence having 100% identity to SEQ ID NOs: 205-220 and 2243-2268.

TABLE 1
Examples NLS Sequences that are used with Cas effectors according to the
present disclosure.

| | | SEQ |
|---|---|---|
| | | ID |
| Source | NLS amino acid sequence | NO: |
| SV40 NLS | PKKKRKV | 205 |
| nucleoplasmin | KRPAATKKAGQAKKKK | 206 |
| bipartite | | |
| c-myc | PAAKRVKLD | 207 |
| c-myc | RQRRNELKRSP | 208 |
| hnRNPA1 M9 | NQSSNFGPMKGGNFGGRSSGPYGGGGQYFAKPRNQGGY | 209 |
| Importin-alpha | RMRIZFKNKGKDTAELRRRRVEVSVELRKAKKDEQILKRR | 210 |
| IBB domain | NV | |
| Myoma T | VSRKRPRP | 211 |
| protein | | |
| Myoma T | PPKKARED | 212 |
| protein | | |
| p53 | PQPKKKPL | 213 |
| mouse c-abl IV | SALIKKKKKMAP | 214 |
| influenza virus | DRLRR | 215 |
| NS1 | | |
| influenza virus | PKQKKRK | 216 |
| NS1 | | |
| Hepatitis virus | RKLKKKIKKL | 217 |
| delta antigen | | |
| mouse Mx1 | REKKKFLKRR | 218 |
| protein | | |
| human | KRKGDEVDGVDEVAKKKSKK | 219 |
| poly(ADP- | | |
| ribose) | | |
| polymerase | | |
| steroid hormone | RKCLQAGMNLEARKTKK | 220 |
| receptors | | |
| glucocorticoid | | |
| 5′ SV40 NLS | MAPKKKRKVGGGGS | 2243 |
| 3′ SV40 1 NLS | SGGAPKKKRKV | 2244 |
| 3′ SV40 2 NLS | SGGAPKKKRKV | 2244 |
| 3′ SV40 3 NLS | SGGAPKKKRKVSGGAPKKKRKVSGGAPKKKRKV | 2245 |
| 5′ | MKRPAATKKAGQAKKKKGGGGS | 2246 |
| Nucleoplasmin | | |
| NLS | | |
| 3′ | SGGKRPAATKKAGQAKKKK | 2247 |
| Nucleoplasmin 1 | | |
| NLS | | |
| 3′ | SGGKRPAATKKAGQAKKKKSGGKRPAATKKAGQAKKKK | 2248 |
| Nucleoplasmin 2 | | |
| NLS | | |
| 3′ | SGGKRPAATKKAGQAKKKKSGGKRPAATKKAGQAKKK | 2249 |
| Nucleoplasmin 3 | KSGGKRPAATKKAGQAKKKK | |
| NLS | | |
| 5′ c-Myc NLS | MPAAKRVKLDGGGGS | 2250 |
| 3′ c-Myc 1 NLS | SGGPAAKRVKLD | 2251 |
| 3′ c-Myc 2 NLS | SGGPAAKRVKLDSGGPAAKRVKLD | 2252 |
| 3′ c-Myc 3 NLS | SGGPAAKRVKLDSGGPAAKRVKLDSGGPAAKRVKLD | 2253 |
| Synthetic 5′ | MQAAKRPRTTGGGGS | 2254 |
| Class 2-1 NLS | | |
| Synthetic 3′ | SGGQAAKRPRTT | 2255 |
| Class 2-1 1 NLS | | |
| Synthetic 3′ | SGGQAAKRPRTTSGGQAAKRPRTT | 2256 |
| Class 2-1 2 NLS | | |
| Synthetic 5′ | MRAAKRPRTTGGGGS | 2257 |
| Class 2-2 NLS | | |
| Synthetic 3′ | SGGRAAKRPRTT | 2258 |
| Class 2-2 1 NLS | | |
| Synthetic 3′ | SGGRAAKRPRTTSGGRAAKRPRTT | 2259 |
| Class 2-2 2 NLS | | |
| Synthetic 5′ | MRAAKRPRTTGGGGS | 2257 |
| Class 2-2 NLS | | |
| Synthetic 3′ | SGGRAAKRPRTT | 2258 |
| Class 2-2 1 NLS | | |
| Synthetic 3′ | SGGRAAKRPRTTSGGRAAKRPRTT | 2259 |
| Class 2-2 2 NLS | | |
| Synthetic 5′ | MAAAKRSWSMAFGGGGS | 2260 |
| Class 3 NLS | | |
| Synthetic 3′ | MSGGAAAKRSWSMAF | 2261 |
| Class 3 1 NLS | | |
| Synthetic 3′ | MSGGAAAKRSWSMAFMSGGAAAKRSWSMAF | 2262 |
| Class 3 2 NLS | | |
| Synthetic 5′ | MRAAKRKYFAVGGGGS | 2263 |
| Class 4 NLS | | |
| Synthetic 3′ | MSGGRAAKRKYFAV | 2264 |
| Class 4 1 NLS | | |
| Synthetic 3′ | MSGGRAAKRKYFAVMSGGRAAKRKYFAV | 2265 |
| Class 4 2 NLS | | |
| 5′ HCV NLS | MPKPQRKTKRGGGGS | 2266 |
| 3′ HCV NLS | SGGPPRKKRTVV | 2267 |
| 3′ hRNP A1 | SGGFGNYNNQSSNFGPMKGGNFGGRSSGPY | 2268 |
| NLS | | |

In some embodiments, the endonucleases described herein are variants thereof with sequence identity to particular domains. In some embodiments, the domain is an arginine rich domain (e.g., a domain with PF14239 homology), a REC (recognition) domain, a BH (bridge helix) domain, a WED (wedge) domain, a PI (PAM-interacting) domain, a PF14239 homology domain, or any other domain described herein. In some embodiments, residues encompassing one or more of these domains is identified in a protein by alignment to one of the proteins below (e.g., when one of the proteins below and the protein of interest are optimally aligned), wherein the residue boundaries for example domains are described.

TABLE 2
Example domain boundaries for endonucleases described herein

| | | | | Domain w/ | | | | WED |
|---|---|---|---|---|---|---|---|---|
| | RuvC-I | BH | REC | PF14239 homology | RuvC-II | HNH | RuvC-III | and PI |
| MG34 | | | | | | | | |
| -1 | | | | | | | | |
| effect | | | 77- | | 282- | 324- | 460- | 552- |
| or | 1-41 | 42-76 | 281 | 4-65; 123-339 | 323 | 459 | 551 | 747 |
| MG34 | | | | | | | | |
| -9 | | | | | | | | |
| effect | | | 77- | | 281- | 323- | 491- | 583- |
| or | 1-41 | 42-76 | 280 | 4-65; 123-338 | 322 | 490 | 582 | 778 |

### Guide Polynucleotides

Disclosed herein, in certain embodiments, are endonuclease systems comprising (a) an endonuclease disclosed herein, and (b) an engineered guide polynucleotide e.g., a guide ribonucleic acid (gRNA), a single gRNA, or a dual guide RNA. In a polynucleotide when referring to a T, a T means U (Uracil) in RNA and T (Thymine) in DNA.

In some embodiments, the engineered guide polynucleotide is configured to form a complex with the endonuclease. In some cases, the engineered guide polynucleotide comprises a spacer sequence. In some cases, the spacer sequence is configured to hybridize to a target nucleic acid sequence. In some cases, the endonuclease is configured to bind to a protospacer adjacent motif (PAM) sequence.

In some embodiments, the guide polynucleotide (e.g., gRNA) targets a gene or locus in a cell. In some embodiments, the guide polynucleotide targets a gene or locus in a mammalian cell. In some embodiments, the mammalian cell is a pig, a cow, a goat, a sheep, a rodent, a rat, a mouse, a non-human primate, or a human cell. In some embodiments, the target gene or target locus is TRAC. In some embodiments, the target gene or target locus is AAVS1.

In some embodiments, the guide polynucleotide is encoded by any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242 or a sequence having at least 90%, 95%, 97%, 98%, or 99% sequence identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide is encoded by a sequence having at least about 80% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide is encoded by a sequence having at least about 85% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide is encoded by a sequence having at least about 90% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide is encoded by a sequence having at least about 95% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide is encoded by a sequence having at least about 96% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide is encoded by a sequence having at least about 97% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide is encoded by a sequence having at least about 98% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide is encoded by a sequence having at least about 99% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide is encoded by a sequence having 100% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242.

In some embodiments, the guide polynucleotide is encoded by any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242 or a sequence having at least 90%, 95%, 97%, 98%, or 99% sequence identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-224. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide hybridizes or targets a sequence complementary having at least about 80% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide hybridizes or targets a sequence complementary having at least about 85% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide hybridizes or targets a sequence complementary having at least about 90% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide hybridizes or targets a sequence complementary having at least about 95% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide hybridizes or targets a sequence complementary having at least about 96% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide hybridizes or targets a sequence complementary having at least about 97% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide hybridizes or targets a sequence complementary having at least about 98% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide hybridizes or targets a sequence complementary having at least about 99% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242. In some embodiments, the guide polynucleotide hybridizes or targets a sequence complementary having 100% identity to any one of SEQ ID NOs: 199-203, 460-461, 613-616, 669-670, 672-673, 677-974, 1003-1022, 1231-1259, 1327-1328, 1348, 1369-1372, 1376-1391, 1392-1414, and 1470-2242.

In some embodiments, the engineered guide polynucleotide comprises a sequence having at least 90% sequence identity to any one of SEQ ID NOs: 1327-1328, 1348, 1369-1372, 1376-1391, 1392-1414, and 1470-2242. In some embodiments, the guide polynucleotide is encoded by any one of SEQ ID NOs: 1327-1328, 1348, 1369-1372, 1376-1391, 1392-1414, and 1470-2242 or a sequence having at least 90%, 95%, 97%, 98%, 99%, or 100% sequence identity to any one of SEQ ID Nos: 1327-1328, 1348, 1369-1372, 1376-1391, 1392-1414, and 1470-2242.

In some embodiments, the engineered guide polynucleotide comprises a sequence having at least 90% sequence identity to any one of SEQ ID NOs: 1571, 1591, 1592, 1615, 1625, 1651, 1663, 1672, 1709, 1712, 1713, 1728, 1738, 1764, 1809, 1812, 1884, 1821, 1853, 1893, 1846, 1854, 1878, 1886, 1902, 1890, 1847, 1903, 1890, 1957, 1959, 1960, 1961, 1975, 1988, and 2002. In some embodiments, the engineered guide polynucleotide comprises a sequence having at least 90%, 95%, 97%, 98%, 99%, or 100% sequence identity to any one of SEQ ID NOs: 1571, 1591, 1592, 1615, 1625, 1651, 1663, 1672, 1709, 1712, 1713, 1728, 1738, 1764, 1809, 1812, 1884, 1821, 1853, 1893, 1846, 1854, 1878, 1886, 1902, 1890, 1847, 1903, 1890, 1957, 1959, 1960, 1961, 1975, 1988, and 2002.

In some embodiments, the engineered guide polynucleotide comprises a sequence having at least 90% sequence identity to SEQ ID NOs: 1410 or 1960. In some embodiments, the engineered guide polynucleotide comprises a sequence having at least 90%, 95%, 97%, 98%, 99%, or 100% sequence identity to any one of SEQ ID NOs: 1410 or 1960.

In some embodiments, the engineered guide polynucleotide comprises a sequence having at least 90% sequence identity to any one of SEQ ID NOs: 1410, 1412, 1953, 1956, 1960, 1961, 1966, 1970, and 1478. In some embodiments, the engineered guide polynucleotide comprises a sequence having at least 90%, 95%, 97%, 98%, 99%, or 100% sequence identity to any one of SEQ ID NOs: 1410, 1412, 1953, 1956, 1960, 1961, 1966, 1970, and 1478.

In some embodiments, the engineered guide polynucleotide comprises a sequence having at least 90% sequence identity to any one of SEQ ID NOs: 2157, 2159, and 2160. In some embodiments, the engineered guide polynucleotide comprises a sequence having at least 90%, 95%, 97%, 98%, 99%, or 100% sequence identity to any one of SEQ ID NOs: 2157, 2159, and 2160.

In some embodiments, the engineered guide polynucleotide comprises a sequence having at least 90% sequence identity to any one of SEQ ID NOs: 2017, 2022, 2029, 2031, 2032, 2035, 2044, 2045, 2047, 2048, 2073, 2075, 2090, 2195, 2197, 2198, 2199, 2200, and 2202. In some embodiments, the engineered guide polynucleotide comprises a sequence having at least 90%, 95%, 97%, 98%, 99%, or 100% sequence identity to any one of SEQ ID NOs: 2017, 2022, 2029, 2031, 2032, 2035, 2044, 2045, 2047, 2048, 2073, 2075, 2090, 2195, 2197, 2198, 2199, 2200, and 2202.

In some embodiments, the engineered guide polynucleotide comprises a sequence having at least 90% sequence identity to any one of SEQ ID NOs: 2017, 2022, 2026, 2028, 2029, 2031, 2032, 2035, 2044, 2047, 2054, 2073, 2075, 2090, 2195, 2197, 2198, 2199, 2200, 2202, 2206, 2208, 2211, 2212, and 2216. In some embodiments, the engineered guide polynucleotide comprises a sequence having at least 90%, 95%, 97%, 98%, 99%, or 100% sequence identity to any one of SEQ ID NOs: 2017, 2022, 2026, 2028, 2029, 2031, 2032, 2035, 2044, 2047, 2054, 2073, 2075, 2090, 2195, 2197, 2198, 2199, 2200, 2202, 2206, 2208, 2211, 2212, and 2216.

In some embodiments, the engineered guide polynucleotide comprises a sequence having 100% sequence identity to any one of SEQ ID NOs: 1327-1328, 1348, 1369-1372, 1376-1391, 1392-1414, and 1470-2242. In some embodiments, the engineered guide polynucleotide comprises a sequence having at least 90%, 95%, 97%, 98%, 99%, or 100% sequence identity to any one of SEQ ID NOs: 1327-1328, 1348, 1369-1372, 1376-1391, 1392-1414, and 1470-2242.

In some embodiments, the guide polynucleotide (e.g., SEQ ID NOs: 199, 201, 669-670, and 1003-1005) is configured to form a complex with a MG33 nuclease. In some embodiments, the guide polynucleotide is encoded by any one of SEQ ID NOs: 199, 201, 669-670, and 1003-1005 or a sequence having at least 90%, 95%, 97%, 98%, or 99% sequence identity to any one of SEQ ID NOs: 199, 201, 669-670, and 1003-1005. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 199, 201, 669-670, and 1003-1005. In some embodiments, the guide polynucleotide is encoded by a sequence having at least about 80% identity to any one of SEQ ID NOs: 199, 201, 669-670, and 1003-1005. In some embodiments, the guide polynucleotide is encoded by a sequence having at least about 85% identity to any one of SEQ ID NOs: 199, 201, 669-670, and 1003-1005. In some embodiments, the guide polynucleotide is encoded by a sequence having at least about 90% identity to any one of SEQ ID NOs: 199, 201, 669-670, and 1003-1005. In some embodiments, the guide polynucleotide is encoded by a sequence having at least about 95% identity to any one of SEQ ID NOs: 199, 201, 669-670, and 1003-1005. In some embodiments, the guide polynucleotide is encoded by a sequence having at least about 96% identity to any one of SEQ ID NOs: 199, 201, 669-670, and 1003-1005. In some embodiments, the guide polynucleotide is encoded by a sequence having at least about 97% identity to any one of SEQ ID NOs: 199, 201, 669-670, and 1003-1005. In some embodiments, the guide polynucleotide is encoded by a sequence having at least about 98% identity to any one of SEQ ID NOs: 199, 201, 669-670, and 1003-1005. In some embodiments, the guide polynucleotide is encoded by a sequence having at least about 99% identity to any one of SEQ ID NOs: 199, 201, 669-670, and 1003-1005. In some embodiments, the guide polynucleotide is encoded by a sequence having 100% identity to any one of SEQ ID NOs: 199, 201, 669-670, and 1003-1005.

In some embodiments, the guide polynucleotide is encoded by any one of SEQ ID NOs: 199, 201, 669-670, and 1003-1005 or a sequence having at least 90%, 95%, 97%, 98%, or 99% sequence identity to any one of SEQ ID NOs: 199, 201, 669-670, and 1003-1005. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 199, 201, 669-670, and 1003-1005. In some embodiments, the guide polynucleotide hybridizes or targets a sequence complementary having at least about 80% identity to any one of SEQ ID NOs: 199, 201, 669-670, and 1003-1005. In some embodiments, the guide polynucleotide hybridizes or targets a sequence complementary having at least about 85% identity to any one of SEQ ID NOs: 199, 201, 669-670, and 1003-1005. In some embodiments, the guide polynucleotide hybridizes or targets a sequence complementary having at least about 90% identity to any one of SEQ ID NOs: 199, 201, 669-670, and 1003-1005. In some embodiments, the guide polynucleotide hybridizes or targets a sequence complementary having at least about 95% identity to any one of SEQ ID NOs: 199, 201, 669-670, and 1003-1005. In some embodiments, the guide polynucleotide hybridizes or targets a sequence complementary having at least about 96% identity to any one of SEQ ID NOs: 199, 201, 669-670, and 1003-1005. In some embodiments, the guide polynucleotide hybridizes or targets a sequence complementary having at least about 97% identity to any one of SEQ ID NOs: 199, 201, 669-670, and 1003-1005. In some embodiments, the guide polynucleotide hybridizes or targets a sequence complementary having at least about 98% identity to any one of SEQ ID NOs: 199, 201, 669-670, and 1003-1005. In some embodiments, the guide polynucleotide hybridizes or targets a sequence complementary having at least about 99% identity to any one of SEQ ID NOs: 199, 201, 669-670, and 1003-1005. In some embodiments, the guide polynucleotide hybridizes or targets a sequence complementary having 100% identity to any one of SEQ ID NOs: 199, 201, 669-670, and 1003-1005.

In some embodiments, the guide polynucleotide (e.g., SEQ ID NOs: 200, 202, 203, 613-616, 1348, 1369, and 1392-1414) is configured to form a complex with a MG34 nuclease. In some embodiments, the guide polynucleotide is encoded by any one of SEQ ID NOs: 200, 202, 203, 613-616, 1348, 1369, and 1392-1414 or a sequence having at least 90%, 95%, 97%, 98%, or 99% sequence identity to any one of SEQ ID NOs: 200, 202, 203, 613-616, 1348, 1369, and 1392-1414. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 200, 202, 203, 613-616, 1348, 1369, and 1392-1414. In some embodiments, the guide polynucleotide is encoded by a sequence having at least about 80% identity to any one of SEQ ID NOs: 200, 202, 203, 613-616, 1348, 1369, and 1392-1414. In some embodiments, the guide polynucleotide is encoded by a sequence having at least about 85% identity to any one of SEQ ID NOs: 200, 202, 203, 613-616, 1348, 1369, and 1392-1414. In some embodiments, the guide polynucleotide is encoded by a sequence having at least about 90% identity to any one of SEQ ID NOs: 200, 202, 203, 613-616, 1348, 1369, and 1392-1414. In some embodiments, the guide polynucleotide is encoded by a sequence having at least about 95% identity to any one of SEQ ID NOs: 200, 202, 203, 613-616, 1348, 1369, and 1392-1414. In some embodiments, the guide polynucleotide is encoded by a sequence having at least about 96% identity to any one of SEQ ID NOs: 200, 202, 203, 613-616, 1348, 1369, and 1392-1414. In some embodiments, the guide polynucleotide is encoded by a sequence having at least about 97% identity to any one of SEQ ID NOs: 200, 202, 203, 613-616, 1348, 1369, and 1392-1414. In some embodiments, the guide polynucleotide is encoded by a sequence having at least about 98% identity to any one of SEQ ID NOs: 200, 202, 203, 613-616, 1348, 1369, and 1392-1414. In some embodiments, the guide polynucleotide is encoded by a sequence having at least about 99% identity to any one of SEQ ID NOs: 200, 202, 203, 613-616, 1348, 1369, and 1392-1414. In some embodiments, the guide polynucleotide is encoded by a sequence having 100% identity to any one of SEQ ID NOs: 200, 202, 203, 613-616, 1348, 1369, and 1392-1414.

In some embodiments, the guide polynucleotide is encoded by any one of SEQ ID NOs: 1348 and 1392-1414 or a sequence having at least 90%, 95%, 97%, 98%, or 99% sequence identity to any one of SEQ ID NOs: 1348 and 1392-1414. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1348 and 1392-1414. In some embodiments, the guide polynucleotide is encoded by a sequence having at least about 80% identity to any one of SEQ ID NOs: 1348 and 1392-1414. In some embodiments, the guide polynucleotide is encoded by a sequence having at least about 85% identity to any one of SEQ ID NOs: 1348 and 1392-1414. In some embodiments, the guide polynucleotide is encoded by a sequence having at least about 90% identity to any one of SEQ ID NOs: 1348 and 1392-1414. In some embodiments, the guide polynucleotide is encoded by a sequence having at least about 95% identity to any one of SEQ ID NOs: 1348 and 1392-1414. In some embodiments, the guide polynucleotide is encoded by a sequence having at least about 96% identity to any one of SEQ ID NOs: 1348 and 1392-1414. In some embodiments, the guide polynucleotide is encoded by a sequence having at least about 97% identity to any one of SEQ ID NOs: 1348 and 1392-1414. In some embodiments, the guide polynucleotide is encoded by a sequence having at least about 98% identity to any one of SEQ ID NOs: 1348 and 1392-1414. In some embodiments, the guide polynucleotide is encoded by a sequence having at least about 99% identity to any one of SEQ ID NOs: 1348 and 1392-1414. In some embodiments, the guide polynucleotide is encoded by a sequence having 100% identity to any one of SEQ ID NOs: 1348 and 1392-1414.

In some embodiments, the guide polynucleotide hybridizes or targets a sequence complementary having at least about 80% identity to any one of SEQ ID NOs: 200, 202, 203, 613-616, 1348, 1369, and 1392-1414. In some embodiments, the guide polynucleotide hybridizes or targets a sequence complementary having at least about 85% identity to any one of SEQ ID NOs: 200, 202, 203, 613-616, 1348, 1369, and 1392-1414. In some embodiments, the guide polynucleotide hybridizes or targets a sequence complementary having at least about 90% identity to any one of SEQ ID NOs: 200, 202, 203, 613-616, 1348, 1369, and 1392-1414. In some embodiments, the guide polynucleotide hybridizes or targets a sequence complementary having at least about 95% identity to any one of SEQ ID NOs: 200, 202, 203, 613-616, 1348, 1369, and 1392-1414. In some embodiments, the guide polynucleotide hybridizes or targets a sequence complementary having at least about 96% identity to any one of SEQ ID NOs: 200, 202, 203, 613-616, 1348, 1369, and 1392-1414. In some embodiments, the guide polynucleotide hybridizes or targets a sequence complementary having at least about 97% identity to any one of SEQ ID NOs: 200, 202, 203, 613-616, 1348, 1369, and 1392-1414. In some embodiments, the guide polynucleotide hybridizes or targets a sequence complementary having at least about 98% identity to any one of SEQ ID NOs: 200, 202, 203, 613-616, 1348, 1369, and 1392-1414. In some embodiments, the guide polynucleotide hybridizes or targets a sequence complementary having at least about 99% identity to any one of SEQ ID NOs: 200, 202, 203, 613-616, 1348, 1369, and 1392-1414. In some embodiments, the guide polynucleotide hybridizes or targets a sequence complementary having 100% identity to any one of SEQ ID NOs: 200, 202, 203, 613-616, 1348, 1369, and 1392-1414.

In some embodiments, the guide polynucleotide hybridizes or targets a sequence complementary having at least about 80% identity to any one of SEQ ID NOs: 1348 and 1392-1414. In some embodiments, the guide polynucleotide hybridizes or targets a sequence complementary having at least about 85% identity to any one of SEQ ID NOs: 1348 and 1392-1414. In some embodiments, the guide polynucleotide hybridizes or targets a sequence complementary having at least about 90% identity to any one of SEQ ID NOs: 1348 and 1392-1414. In some embodiments, the guide polynucleotide hybridizes or targets a sequence complementary having at least about 95% identity to any one of SEQ ID NOs: 1348 and 1392-1414. In some embodiments, the guide polynucleotide hybridizes or targets a sequence complementary having at least about 96% identity to any one of SEQ ID NOs: 1348 and 1392-1414. In some embodiments, the guide polynucleotide hybridizes or targets a sequence complementary having at least about 97% identity to any one of SEQ ID NOs: 1348 and 1392-1414. In some embodiments, the guide polynucleotide hybridizes or targets a sequence complementary having at least about 98% identity to any one of SEQ ID NOs: 1348 and 1392-1414. In some embodiments, the guide polynucleotide hybridizes or targets a sequence complementary having at least about 99% identity to any one of SEQ ID NOs: 1348 and 1392-1414. In some embodiments, the guide polynucleotide hybridizes or targets a sequence complementary having 100% identity to any one of SEQ ID NOs: 1348 and 1392-1414.

In some embodiments, the guide polynucleotide (e.g., SEQ ID NOs: 460-461, 677-974, 1006-1012, and 1231-1259) is configured to form a complex with a MG35 nuclease. In some embodiments, the guide polynucleotide is encoded by any one of SEQ ID NOs: 460-461, 677-974, 1006-1012, and 1231-1259 or a sequence having at least 90%, 95%, 97%, 98%, or 99% sequence identity to any one of SEQ ID NOs: 460-461, 677-974, 1006-1012, and 1231-1259. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 460-461, 677-974, 1006-1012, and 1231-1259. In some embodiments, the guide polynucleotide is encoded by a sequence having at least about 80% identity to any one of SEQ ID NOs: 460-461, 677-974, 1006-1012, and 1231-1259. In some embodiments, the guide polynucleotide is encoded by a sequence having at least about 85% identity to any one of SEQ ID NOs: 460-461, 677-974, 1006-1012, and 1231-1259. In some embodiments, the guide polynucleotide is encoded by a sequence having at least about 90% identity to any one of SEQ ID NOs: 460-461, 677-974, 1006-1012, and 1231-1259. In some embodiments, the guide polynucleotide is encoded by a sequence having at least about 95% identity to any one of SEQ ID NOs: 460-461, 677-974, 1006-1012, and 1231-1259. In some embodiments, the guide polynucleotide is encoded by a sequence having at least about 96% identity to any one of SEQ ID NOs: 460-461, 677-974, 1006-1012, and 1231-1259. In some embodiments, the guide polynucleotide is encoded by a sequence having at least about 97% identity to any one of SEQ ID NOs: 460-461, 677-974, 1006-1012, and 1231-1259. In some embodiments, the guide polynucleotide is encoded by a sequence having at least about 98% identity to any one of SEQ ID NOs: 460-461, 677-974, 1006-1012, and 1231-1259. In some embodiments, the guide polynucleotide is encoded by a sequence having at least about 99% identity to any one of SEQ ID NOs: 460-461, 677-974, 1006-1012, and 1231-1259. In some embodiments, the guide polynucleotide is encoded by a sequence having 100% identity to any one of SEQ ID NOs: 460-461, 677-974, 1006-1012, and 1231-1259.

In some embodiments, the guide polynucleotide hybridizes or targets a sequence complementary having at least about 80% identity to any one of SEQ ID NOs: 460-461, 677-974, 1006-1012, 1231-1259. In some embodiments, the guide polynucleotide hybridizes or targets a sequence complementary having at least about 85% identity to any one of SEQ ID NOs: 460-461, 677-974, 1006-1012, and 1231-1259. In some embodiments, the guide polynucleotide hybridizes or targets a sequence complementary having at least about 90% identity to any one of SEQ ID NOs: 460-461, 677-974, 1006-1012, and 1231-1259. In some embodiments, the guide polynucleotide hybridizes or targets a sequence complementary having at least about 95% identity to any one of SEQ ID NOs: 460-461, 677-974, 1006-1012, and 1231-1259. In some embodiments, the guide polynucleotide hybridizes or targets a sequence complementary having at least about 96% identity to any one of SEQ ID NOs: 460-461, 677-974, 1006-1012, and 1231-1259. In some embodiments, the guide polynucleotide hybridizes or targets a sequence complementary having at least about 97% identity to any one of SEQ ID NOs: 460-461, 677-974, 1006-1012, and 1231-1259. In some embodiments, the guide polynucleotide hybridizes or targets a sequence complementary having at least about 98% identity to any one of SEQ ID NOs: 460-461, 677-974, 1006-1012, and 1231-1259. In some embodiments, the guide polynucleotide hybridizes or targets a sequence complementary having at least about 99% identity to any one of SEQ ID NOs: 460-461, 677-974, 1006-1012, and 1231-1259. In some embodiments, the guide polynucleotide hybridizes or targets a sequence complementary having 100% identity to any one of SEQ ID NOs: 460-461, 677-974, 1006-1012, and 1231-1259.

In some embodiments, the guide polynucleotide targets a sequence having at least about 80% identity to any one of SEQ ID NOs: 2270-2330. In some embodiments, the guide polynucleotide hybridizes or targets a sequence having at least about 85% identity to any one of SEQ ID NOs: 2270-2330. In some embodiments, the guide polynucleotide hybridizes or targets a sequence having at least about 90% identity to any one of SEQ ID NOs: 2270-2330. In some embodiments, the guide polynucleotide hybridizes or targets a sequence having at least about 95% identity to any one of SEQ ID NOs: 2270-2330. In some embodiments, the guide polynucleotide hybridizes or targets a sequence having at least about 96% identity to any one of SEQ ID NOs: 2270-2330. In some embodiments, the guide polynucleotide hybridizes or targets a sequence having at least about 97% identity to any one of SEQ ID NOs: 2270-2330. In some embodiments, the guide polynucleotide hybridizes or targets a sequence having at least about 98% identity to any one of SEQ ID NOs: 2270-2330. In some embodiments, the guide polynucleotide hybridizes or targets a sequence having at least about 99% identity to any one of SEQ ID NOs: 2270-2330. In some embodiments, the guide polynucleotide hybridizes or targets a sequence having 100% identity to any one of SEQ ID NOs: 2270-2330.

In some embodiments, the guide polynucleotide (e.g., SEQ ID NOs: 672-673, 1013-1022, 1327-1328, 1370-1372, and 1376-1391) is configured to form a complex with a MG102 nuclease. In some embodiments, the guide polynucleotide is encoded by any one of SEQ ID NOs: 672-673, 1013-1022, 1327-1328, 1370-1372, and 1376-1391 or a sequence having at least 90%, 95%, 97%, 98%, or 99% sequence identity to any one of SEQ ID NOs: 672-673, 1013-1022, 1327-1328, 1370-1372, and 1376-1391. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 672-673, 1013-1022, 1327-1328, 1370-1372, and 1376-1391. In some embodiments, the guide polynucleotide is encoded by a sequence having at least about 80% identity to any one of SEQ ID NOs: 672-673, 1013-1022, 1327-1328, 1370-1372, and 1376-1391. In some embodiments, the guide polynucleotide is encoded by a sequence having at least about 85% identity to any one of SEQ ID NOs: 672-673, 1013-1022, 1327-1328, 1370-1372, and 1376-1391. In some embodiments, the guide polynucleotide is encoded by a sequence having at least about 90% identity to any one of SEQ ID NOs: 672-673, 1013-1022, 1327-1328, 1370-1372, and 1376-1391. In some embodiments, the guide polynucleotide is encoded by a sequence having at least about 95% identity to any one of SEQ ID NOs: 672-673, 1013-1022, 1327-1328, 1370-1372, and 1376-1391. In some embodiments, the guide polynucleotide is encoded by a sequence having at least about 96% identity to any one of SEQ ID NOs: 672-673, 1013-1022, 1327-1328, 1370-1372, and 1376-1391. In some embodiments, the guide polynucleotide is encoded by a sequence having at least about 97% identity to any one of SEQ ID NOs: 672-673, 1013-1022, 1327-1328, 1370-1372, and 1376-1391. In some embodiments, the guide polynucleotide is encoded by a sequence having at least about 98% identity to any one of SEQ ID NOs: 672-673, 1013-1022, 1327-1328, 1370-1372, and 1376-1391. In some embodiments, the guide polynucleotide is encoded by a sequence having at least about 99% identity to any one of SEQ ID NOs: 672-673, 1013-1022, 1327-1328, 1370-1372, and 1376-1391. In some embodiments, the guide polynucleotide is encoded by a sequence having 100% identity to any one of SEQ ID NOs: 672-673, 1013-1022, 1327-1328, 1370-1372, and 1376-1391.

In some embodiments, the guide polynucleotide is encoded by any one of SEQ ID NOs: 1376-1391 or a sequence having at least 90%, 95%, 97%, 98%, or 99% sequence identity to any one of SEQ ID NOs: 1376-1391. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1376-1391. In some embodiments, the guide polynucleotide is encoded by a sequence having at least about 80% identity to any one of SEQ ID NOs: 1376-1391. In some embodiments, the guide polynucleotide is encoded by a sequence having at least about 85% identity to any one of SEQ ID NOs: 1376-1391. In some embodiments, the guide polynucleotide is encoded by a sequence having at least about 90% identity to any one of SEQ ID NOs: 1376-1391. In some embodiments, the guide polynucleotide is encoded by a sequence having at least about 95% identity to any one of SEQ ID NOs: 1376-1391. In some embodiments, the guide polynucleotide is encoded by a sequence having at least about 96% identity to any one of SEQ ID NOs: 1376-1391. In some embodiments, the guide polynucleotide is encoded by a sequence having at least about 97% identity to any one of SEQ ID NOs: 1376-1391. In some embodiments, the guide polynucleotide is encoded by a sequence having at least about 98% identity to any one of SEQ ID NOs: 1376-1391. In some embodiments, the guide polynucleotide is encoded by a sequence having at least about 99% identity to any one of SEQ ID NOs: 1376-1391. In some embodiments, the guide polynucleotide is encoded by a sequence having 100% identity to any one of SEQ ID NOs: 1376-1391.

In some embodiments, the guide polynucleotide hybridizes or targets a sequence complementary having at least about 80% identity to any one of SEQ ID NOs: 672-673, 1013-1022, 1327-1328, 1370-1372, and 1376-1391. In some embodiments, the guide polynucleotide hybridizes or targets a sequence complementary having at least about 85% identity to any one of SEQ ID NOs: 672-673, 1013-1022, 1327-1328, 1370-1372, and 1376-1391. In some embodiments, the guide polynucleotide hybridizes or targets a sequence complementary having at least about 90% identity to any one of SEQ ID NOs: 672-673, 1013-1022, 1327-1328, 1370-1372, and 1376-1391. In some embodiments, the guide polynucleotide hybridizes or targets a sequence complementary having at least about 95% identity to any one of SEQ ID NOs: 672-673, 1013-1022, 1327-1328, 1370-1372, and 1376-1391. In some embodiments, the guide polynucleotide hybridizes or targets a sequence complementary having at least about 96% identity to any one of SEQ ID NOs: 672-673, 1013-1022, 1327-1328, 1370-1372, and 1376-1391. In some embodiments, the guide polynucleotide hybridizes or targets a sequence complementary having at least about 97% identity to any one of SEQ ID NOs: 672-673, 1013-1022, 1327-1328, 1370-1372, and 1376-1391. In some embodiments, the guide polynucleotide hybridizes or targets a sequence complementary having at least about 98% identity to any one of SEQ ID NOs: 672-673, 1013-1022, 1327-1328, 1370-1372, and 1376-1391. In some embodiments, the guide polynucleotide hybridizes or targets a sequence complementary having at least about 99% identity to any one of SEQ ID NOs: 672-673, 1013-1022, 1327-1328, 1370-1372, and 1376-1391. In some embodiments, the guide polynucleotide hybridizes or targets a sequence complementary having 100% identity to any one of SEQ ID NOs: 672-673, 1013-1022, 1327-1328, 1370-1372, and 1376-1391.

In some embodiments, the guide polynucleotide hybridizes or targets a sequence complementary having at least about 80% identity to any one of SEQ ID NOs: 1376-1391. In some embodiments, the guide polynucleotide hybridizes or targets a sequence complementary having at least about 85% identity to any one of SEQ ID NOs: 1376-1391. In some embodiments, the guide polynucleotide hybridizes or targets a sequence complementary having at least about 90% identity to any one of SEQ ID NOs: 1376-1391. In some embodiments, the guide polynucleotide hybridizes or targets a sequence complementary having at least about 95% identity to any one of SEQ ID NOs: 1376-1391. In some embodiments, the guide polynucleotide hybridizes or targets a sequence complementary having at least about 96% identity to any one of SEQ ID NOs: 1376-1391. In some embodiments, the guide polynucleotide hybridizes or targets a sequence complementary having at least about 97% identity to any one of SEQ ID NOs: 1376-1391. In some embodiments, the guide polynucleotide hybridizes or targets a sequence complementary having at least about 98% identity to any one of SEQ ID NOs: 1376-1391. In some embodiments, the guide polynucleotide hybridizes or targets a sequence complementary having at least about 99% identity to any one of SEQ ID NOs: 1376-1391. In some embodiments, the guide polynucleotide hybridizes or targets a sequence complementary having 100% identity to any one of SEQ ID NOs: 1376-1391.

In some embodiments, the target gene is TRAC. In some embodiments, the guide polynucleotide is encoded by any one of SEQ ID NOs: 1083-1086, 1123-1144, and 1167-1168 or a sequence having at least 90%, 95%, 97%, 98%, or 99% sequence identity to any one of SEQ ID NOs: 1083-1086, 1123-1144, and 1167-1168. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1083-1086, 1123-1144, and 1167-1168. In some embodiments, the guide polynucleotide is encoded by a sequence having at least about 80% identity to any one of SEQ ID NOs: 1083-1086, 1123-1144, and 1167-1168. In some embodiments, the guide polynucleotide is encoded by a sequence having at least about 85% identity to any one of SEQ ID NOs: 1083-1086, 1123-1144, and 1167-1168. In some embodiments, the guide polynucleotide is encoded by a sequence having at least about 90% identity to any one of SEQ ID NOs: 1083-1086, 1123-1144, and 1167-1168. In some embodiments, the guide polynucleotide is encoded by a sequence having at least about 95% identity to any one of SEQ ID NOs: 1083-1086, 1123-1144, and 1167-1168. In some embodiments, the guide polynucleotide is encoded by a sequence having at least about 96% identity to any one of SEQ ID NOs: 1083-1086, 1123-1144, and 1167-1168. In some embodiments, the guide polynucleotide is encoded by a sequence having at least about 97% identity to any one of SEQ ID NOs: 1083-1086, 1123-1144, and 1167-1168. In some embodiments, the guide polynucleotide is encoded by a sequence having at least about 98% identity to any one of SEQ ID NOs: 1083-1086, 1123-1144, and 1167-1168. In some embodiments, the guide polynucleotide is encoded by a sequence having at least about 99% identity to any one of SEQ ID NOs: 1083-1086, 1123-1144, and 1167-1168. In some embodiments, the guide polynucleotide is encoded by a sequence having 100% identity to any one of SEQ ID NOs: 1083-1086, 1123-1144, and 1167-1168.

In some embodiments, the guide polynucleotide hybridizes or targets a sequence complementary to a target nucleic acid sequence within the TRAC gene or within an intron of an endogenous gene. In some embodiments, the guide polynucleotide hybridizes or targets a sequence complementary to any one of SEQ ID NOs: 1083-1086, 1123-1144, and 1167-1168 or a sequence having at least 90%, 95%, 97%, 98%, or 99% sequence identity to any one of SEQ ID NOs: 1083-1086, 1123-1144, and 1167-1168. In some embodiments, the guide polynucleotide hybridizes or targets a sequence complementary to a sequence having at least about 80% identity to any one of SEQ ID NOs: 1083-1086, 1123-1144, and 1167-1168. In some embodiments, the guide polynucleotide hybridizes or targets a sequence complementary to a sequence having at least about 85% identity to any one of SEQ ID NOs: 1083-1086, 1123-1144, and 1167-1168. In some embodiments, the guide polynucleotide hybridizes or targets a sequence complementary to a sequence having at least about 90% identity to any one of SEQ ID NOs: 1083-1086, 1123-1144, and 1167-1168. In some embodiments, the guide polynucleotide hybridizes or targets a sequence complementary to a sequence having at least about 95% identity to any one of SEQ ID NOs: 1083-1086, 1123-1144, and 1167-1168. In some embodiments, the guide polynucleotide hybridizes or targets a sequence complementary to a sequence having at least about 96% identity to any one of SEQ ID NOs: 1083-1086, 1123-1144, and 1167-1168. In some embodiments, the guide polynucleotide hybridizes or targets a sequence complementary to a sequence having at least about 97% identity to any one of SEQ ID NOs: 1083-1086, 1123-1144, and 1167-1168. In some embodiments, the guide polynucleotide hybridizes or targets a sequence complementary to a sequence having at least about 98% identity to any one of SEQ ID NOs: 1083-1086, 1123-1144, and 1167-1168. In some embodiments, the guide polynucleotide hybridizes or targets a sequence complementary to a sequence having at least about 99% identity to any one of SEQ ID NOs: 1083-1086, 1123-1144, and 1167-1168. In some embodiments, the guide polynucleotide hybridizes or targets a sequence complementary to a sequence having 100% identity to any one of SEQ ID NOs: 1083-1086, 1123-1144, and 1167-1168.

In some embodiments, the guide polynucleotide hybridizes or targets a sequence within the TRAC gene or within an intron of an endogenous gene. In some embodiments, the guide polynucleotide hybridizes or targets a sequence according to any one of SEQ ID NOs: 1079-1082, 1145-1166, and 1169-1170 or a sequence having at least 90%, 95%, 97%, 98%, or 99% sequence identity to any one of SEQ ID NOs: 1079-1082, 1145-1166, and 1169-1170. In some embodiments, the guide polynucleotide hybridizes or targets a sequence having at least about 80% identity to any one of SEQ ID NOs: 1079-1082, 1145-1166, and 1169-1170. In some embodiments, the guide polynucleotide hybridizes or targets a sequence having at least about 85% identity to any one of SEQ ID NOs: 1079-1082, 1145-1166, and 1169-1170. In some embodiments, the guide polynucleotide hybridizes or targets a sequence having at least about 90% identity to any one of SEQ ID NOs: 1079-1082, 1145-1166, and 1169-1170. In some embodiments, the guide polynucleotide hybridizes or targets a sequence having at least about 95% identity to any one of SEQ ID NOs: 1079-1082, 1145-1166, and 1169-1170. In some embodiments, the guide polynucleotide hybridizes or targets a sequence having at least about 96% identity to any one of SEQ ID NOs: 1079-1082, 1145-1166, and 1169-1170. In some embodiments, the guide polynucleotide hybridizes or targets a sequence having at least about 97% identity to any one of SEQ ID NOs: 1079-1082, 1145-1166, and 1169-1170. In some embodiments, the guide polynucleotide hybridizes or targets a sequence having at least about 98% identity to any one of SEQ ID NOs: 1079-1082, 1145-1166, and 1169-1170. In some embodiments, the guide polynucleotide hybridizes or targets a sequence having at least about 99% identity to any one of SEQ ID NOs: 1079-1082, 1145-1166, and 1169-1170. In some embodiments, the guide polynucleotide hybridizes or targets a sequence having 100% identity to any one of SEQ ID NOs: 1079-1082, 1145-1166, and 1169-1170.

some embodiments, the target gene is AAVS1. In some embodiments, the guide polynucleotide is encoded by any one of SEQ ID NOs: 1087-1104 or a sequence having at least 90%, 95%, 97%, 98%, or 99% sequence identity to any one of SEQ ID NOs: 1087-1104. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1087-1104. In some embodiments, the guide polynucleotide is encoded by a sequence having at least about 80% identity to any one of SEQ ID NOs: 1087-1104. In some embodiments, the guide polynucleotide is encoded by a sequence having at least about 85% identity to any one of SEQ ID NOs: 1087-1104. In some embodiments, the guide polynucleotide is encoded by a sequence having at least about 90% identity to any one of SEQ ID NOs: 1087-1104. In some embodiments, the guide polynucleotide is encoded by a sequence having at least about 95% identity to any one of SEQ ID NOs: 1087-1104. In some embodiments, the guide polynucleotide is encoded by a sequence having at least about 96% identity to any one of SEQ ID NOs: 1087-1104. In some embodiments, the guide polynucleotide is encoded by a sequence having at least about 97% identity to any one of SEQ ID NOs: 1087-1104. In some embodiments, the guide polynucleotide is encoded by a sequence having at least about 98% identity to any one of SEQ ID NOs: 1087-1104. In some embodiments, the guide polynucleotide is encoded by a sequence having at least about 99% identity to any one of SEQ ID NOs: 1087-1104. In some embodiments, the guide polynucleotide is encoded by a sequence having 100% identity to any one of SEQ ID NOs: 1087-1104.

In some embodiments, the guide polynucleotide hybridizes or targets a sequence complementary to a target nucleic acid sequence within the AAVS1 gene or within an intron of an endogenous gene. In some embodiments, the guide polynucleotide hybridizes or targets a sequence complementary to any one of SEQ ID NOs: 1087-1104 or a sequence having at least 90%, 95%, 97%, 98%, or 99% sequence identity to any one of SEQ ID NOs: 1087-1104. In some embodiments, the guide polynucleotide hybridizes or targets a sequence complementary to a sequence having at least about 80% identity to any one of SEQ ID NOs: 1087-1104. In some embodiments, the guide polynucleotide hybridizes or targets a sequence complementary to a sequence having at least about 85% identity to any one of SEQ ID NOs: 1087-1104. In some embodiments, the guide polynucleotide hybridizes or targets a sequence complementary to a sequence having at least about 90% identity to any one of SEQ ID NOs: 1087-1104. In some embodiments, the guide polynucleotide hybridizes or targets a sequence complementary to a sequence having at least about 95% identity to any one of SEQ ID NOs: 1087-1104. In some embodiments, the guide polynucleotide hybridizes or targets a sequence complementary to a sequence having at least about 96% identity to any one of SEQ ID NOs: 1087-1104. In some embodiments, the guide polynucleotide hybridizes or targets a sequence complementary to a sequence having at least about 97% identity to any one of SEQ ID NOs: 1087-1104. In some embodiments, the guide polynucleotide hybridizes or targets a sequence complementary to a sequence having at least about 98% identity to any one of SEQ ID NOs: 1087-1104. In some embodiments, the guide polynucleotide hybridizes or targets a sequence complementary to a sequence having at least about 99% identity to any one of SEQ ID NOs: 1087-1104. In some embodiments, the guide polynucleotide hybridizes or targets a sequence complementary to a sequence having 100% identity to any one of SEQ ID NOs: 1087-1104.

In some embodiments, the guide polynucleotide hybridizes or targets a sequence within the AAVS1 gene or within an intron of an endogenous gene. In some embodiments, the guide polynucleotide hybridizes or targets a sequence according to any one of SEQ ID NOs: 1105-1122 or a sequence having at least 90%, 95%, 97%, 98%, or 99% sequence identity to any one of SEQ ID NOs: 1105-1122. In some embodiments, the guide polynucleotide hybridizes or targets a sequence having at least about 80% identity to any one of SEQ ID NOs: 1105-1122. In some embodiments, the guide polynucleotide hybridizes or targets a sequence having at least about 85% identity to any one of SEQ ID NOs: 1105-1122. In some embodiments, the guide polynucleotide hybridizes or targets a sequence having at least about 90% identity to any one of SEQ ID NOs: 1105-1122. In some embodiments, the guide polynucleotide hybridizes or targets a sequence having at least about 95% identity to any one of SEQ ID NOs: 1105-1122. In some embodiments, the guide polynucleotide hybridizes or targets a sequence having at least about 96% identity to any one of SEQ ID NOs: 1105-1122. In some embodiments, the guide polynucleotide hybridizes or targets a sequence having at least about 97% identity to any one of SEQ ID NOs: 1105-1122. In some embodiments, the guide polynucleotide hybridizes or targets a sequence having at least about 98% identity to any one of SEQ ID NOs: 1105-1122. In some embodiments, the guide polynucleotide hybridizes or targets a sequence having at least about 99% identity to any one of SEQ ID NOs: 1105-1122. In some embodiments, the guide polynucleotide hybridizes or targets a sequence having 100% identity to any one of SEQ ID NOs: 1105-1122.

In some embodiments, the systems provided herein comprise one or more guide polynucleotides. In some embodiments, the guide polynucleotide comprises a sense sequence. In some embodiments, the guide polynucleotide comprises an anti-sense sequence. In some embodiments, the guide polynucleotide comprises nucleotide sequences other than the region complementary to or substantially complementary to a region of a target sequence. For example, a crRNA is part or considered part of a guide polynucleotide, or is comprised in a guide polynucleotide, e.g., a crRNA:tracrRNA chimera.

In some embodiments, the guide polynucleotide comprises synthetic nucleotides or modified nucleotides. In some embodiments, the guide polynucleotide comprises one or more inter-nucleoside linkers modified from the natural phosphodiester. In some embodiments, all of the inter-nucleoside linkers of the guide polynucleotide, or contiguous nucleotide sequence thereof, are modified. For example, in some embodiments, the inter nucleoside linkage comprises Sulphur (S), such as a phosphorothioate inter-nucleoside linkage. In some embodiments, the guide polynucleotide comprises greater than about 10%, 25%, 50%, 75%, or 90% modified inter-nucleoside linkers. In some embodiments, the guide polynucleotide comprises 1, 2, 3, 4, 5, 6, 7, 8, 9, 10, or more than 10 modified inter-nucleoside linkers (e.g., phosphorothioate inter-nucleoside linkage).

In some embodiments, the guide polynucleotide comprises modifications to a ribose sugar or nucleobase. In some embodiments, the guide polynucleotide comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide polynucleotide comprises one or more modified sugars. In some embodiments, the sugar modifications comprise modifications made by altering the substituent groups on the ribose ring to groups other than hydrogen, or the 2′—OH group naturally found in DNA and RNA nucleosides. In some embodiments, substituents are introduced at the 2′, 3′, 4′, 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide polynucleotide comprises one or more modified sugars. In some embodiments, the guide polynucleotide comprises only modified sugars. In some embodiments, the guide polynucleotide comprises greater than about 10%, 25%, 50%, 75%, or 90% modified sugars. In some embodiments, the modified sugar is a bicyclic sugar. In some embodiments, the modified sugar comprises a 2′-O-methyl. In some embodiments, the modified sugar comprises a 2′-fluoro. In some embodiments, the modified sugar comprises a 2′-O-methoxyethyl group. In some embodiments, the guide polynucleotide comprises 1, 2, 3, 4, 5, 6, 7, 8, 9, 10, or more than 10 modified sugars (e.g., comprising a 2′-O-methyl or 2′-fluoro).

In some embodiments, the guide polynucleotide comprises both inter-nucleoside linker modifications and nucleoside modifications. In some embodiments, the guide polynucleotide comprises greater than about 10%, 25%, 50%, 75%, or 90% modified inter-nucleoside linkers and greater than about 10%, 25%, 50%, 75%, or 90% modified sugars. In some embodiments, the guide polynucleotide comprises 1, 2, 3, 4, 5, 6, 7, 8, 9, 10, or more than 10 modified inter-nucleoside linkers (e.g., phosphorothioate inter-nucleoside linkage) and 1, 2, 3, 4, 5, 6, 7, 8, 9, 10, or more than 10 modified sugars (e.g., comprising a 2′-O-methyl or 2′-fluoro).

In some cases, the guide polynucleotide comprises a sequence complementary to a eukaryotic, fungal, plant, mammalian, or human genomic polynucleotide sequence. In some cases, the guide polynucleotide comprises a sequence complementary to a eukaryotic genomic polynucleotide sequence. In some cases, the guide polynucleotide comprises a sequence complementary to a fungal genomic polynucleotide sequence. In some cases, the guide polynucleotide comprises a sequence complementary to a plant genomic polynucleotide sequence. In some cases, the guide polynucleotide comprises a sequence complementary to a mammalian genomic polynucleotide sequence. In some cases, the guide polynucleotide comprises a sequence complementary to a human genomic polynucleotide sequence.

In some embodiments, the guide polynucleotide is 30-250 nucleotides in length. In some embodiments, the guide polynucleotide is more than 90 nucleotides in length. In some embodiments, the guide polynucleotide is less than 245 nucleotides in length. In some embodiments, the guide polynucleotide is 30, 40, 50, 60, 70, 80, 90, 100, 120, 140, 160, 180, 200, 220, 240, or more than 240 nucleotides in length. In some embodiments, the guide polynucleotide is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

In some embodiments, the guide RNA structure comprises an RNA sequence predicted to comprise a hairpin. In some embodiments, the hair pin comprises a stem and a loop.

In some embodiments, the stem comprises at least 12 pairs, at least 14 pairs, at least 16 pairs or at least 18 pairs of ribonucleotides. In some embodiments, the stem comprises at least 12 pairs of ribonucleotides. In some embodiments, the stem comprises at least 14 pairs of ribonucleotides. In some embodiments, the stem comprises at least 16 pairs of ribonucleotides. In some embodiments, the stem comprises at least 18 pairs of ribonucleotides.

In some embodiments, the guide RNA structure further comprises a second stem and a second loop.

In some embodiments, the second stem comprises at least 5 pairs, at least 6 pairs, at least 7 pairs, at least 8 pairs, at least 9 pairs or at least 10 pairs of ribonucleotides. In some embodiments, the second stem comprises at least 5 pairs of ribonucleotides. In some embodiments, the second stem comprises at least 6 pairs of ribonucleotides. In some embodiments, the second stem comprises at least 7 pairs of ribonucleotides. In some embodiments, the second stem comprises at least 8 pairs of ribonucleotides. In some embodiments, the second stem comprises at least 9 pairs of ribonucleotides. In some embodiments, the second stem comprises at least 10 pairs of ribonucleotides.

In some embodiments, the guide RNA structure further comprises an RNA structure and this RNA structure comprises at least two hairpins.

In some embodiments, the guide ribonucleic acid sequence is complementary to a prokaryotic, bacterial, archaeal, eukaryotic, fungal, plant, mammalian, or human genomic sequence. In some embodiments, the guide ribonucleic acid sequence is complementary to a prokaryotic genomic sequence. In some embodiments, the guide ribonucleic acid sequence is complementary to a bacterial genomic sequence. In some embodiments, the guide ribonucleic acid sequence is complementary to an archaeal genomic sequence. In some embodiments, the guide ribonucleic acid sequence is complementary to a eukaryotic genomic sequence. In some embodiments, the guide ribonucleic acid sequence is complementary to a fungal genomic sequence. In some embodiments, the guide ribonucleic acid sequence is complementary to a plant genomic sequence. In some embodiments, the guide ribonucleic acid sequence is complementary to a mammalian genomic sequence. In some embodiments, the guide ribonucleic acid sequence is complementary to a human genomic sequence.

### MG Endonuclease Systems

Described herein, in certain embodiments, are engineered nuclease systems comprising an endonuclease and an engineered guide polynucleotide configured to form a complex with the endonuclease and to hybridize to a target nucleic acid sequence.

In some embodiments, the engineered nuclease system comprises an endonuclease comprising a sequence with at least 70% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 75% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 75% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 80% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 81% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 82% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 83% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 84% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 85% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 86% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 87% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 88% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 89% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 90% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 91% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 92% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 93% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 94% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 95% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 96% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 97% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 98% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 99% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 100% sequence identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease.

Described herein, in certain embodiments, are engineered nuclease systems comprising: an endonuclease comprising a sequence having at least 80% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 80% identity to any one of SEQ ID NOs: 1350-1368 and 1415-1440. In some embodiments, the endonuclease comprises a sequence having at least 80% identity to any one of SEQ ID NOs: 1324 and 1329-1346.

Described herein, in certain embodiments, are engineered nuclease systems comprising: an endonuclease comprising a sequence having at least 90% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 90% identity to 1350-1368 and 1415-1440. In some embodiments, the endonuclease comprises a sequence having at least 90% identity to any one of SEQ ID NOs: 1324 and 1329-1346.

Described herein, in certain embodiments, are engineered nuclease systems comprising an endonuclease comprising a sequence having at least 95% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 95% identity to 1350-1368 and 1415-1440. In some embodiments, the endonuclease comprises a sequence having at least 95% identity to any one of SEQ ID NOs: 1324 and 1329-1346.

Described herein, in certain embodiments, are engineered nuclease systems comprising an endonuclease comprising a sequence having at least 99% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence. In some embodiments, the endonuclease comprises a sequence having at least 99% identity to 1350-1368 and 1415-1440. In some embodiments, the endonuclease comprises a sequence having at least 99% identity to any one of SEQ ID NOs: 1324 and 1329-1346.

Described herein, in certain embodiments, are engineered nuclease systems comprising an endonuclease comprising a sequence having 100% sequence identity to any one of SEQ ID NOs: 1323-1324, 1329-1347, 1350-1368, and 1415-1440; and an engineered guide polynucleotide that forms a complex with the endonuclease and hybridizes to a target nucleic acid sequence. In some embodiments, the endonuclease comprises a sequence having 100% sequence identity to any one of SEQ ID NOs: 1347, 1350-1368, and 1415-1440. In some embodiments, the endonuclease comprises a sequence having 100% identity to any one of SEQ ID NOs: 1323, 1324 and 1329-1346.

In some embodiments, the engineered nuclease system comprises an endonuclease comprising a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 70% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 75% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 75% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 80% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 81% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 82% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 83% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 84% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 85% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 86% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 87% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 88% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 89% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 90% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 91% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 92% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 93% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 94% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 95% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 96% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 97% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 98% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 99% sequence identity to any one of SEQ ID NOs: 1322-1324, 1329-1347, 1350-1368, and 1415-1440 and an engineered guide polynucleotide configured to form a complex with the endonuclease. In some embodiments, the engineered nuclease system comprises an endonuclease comprising a sequence with at least 100% sequence identity to any one of SEQ ID NOs: 1322-1324, 1329-1347, 1350-1368, and 1415-1440 and an engineered guide polynucleotide configured to form a complex with the endonuclease.

In some embodiments, the engineered nuclease system comprises an endonuclease comprising a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 70% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 75% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 75% sequence identity to any one of SEQ ID NOs: 1324, 1329-1346, 1350-1368, and 1415-1440 and an engineered guide polynucleotide configured to form a complex with the endonuclease. In some embodiments, the engineered nuclease system comprises an endonuclease comprising a sequence with at least 80% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 81% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 82% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 83% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 84% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 85% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 86% sequence identity to any one of SEQ ID NOs: 21324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 87% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 88% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 89% sequence identity to any one of SEQ ID NOs: 21324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 90% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 91% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 92% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 93% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 94% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 95% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 96% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 97% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 98% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 99% sequence identity to any one of SEQ ID NOs: 1324, 1329-1346, 1347, 1350-1368, and 1415-1440 and an engineered guide polynucleotide configured to form a complex with the endonuclease. In some embodiments, the engineered nuclease system comprises an endonuclease comprising a sequence with at least 100% sequence identity to any one of SEQ ID NOs: 1324, 1329-1346, 1347, 1350-1368, and 1415-1440 and an engineered guide polynucleotide configured to form a complex with the endonuclease.

In some embodiments, the engineered nuclease system comprises an endonuclease comprising a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 70% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 75% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 75% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 80% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 81% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 82% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 83% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 84% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 85% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 86% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 87% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 88% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 89% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 90% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 91% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 92% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 93% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 94% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 95% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 96% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 97% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 98% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 99% sequence identity to any one of SEQ ID NOs: 25-198, 221-459, 489-580, 617-668, and 674-675 and an engineered guide polynucleotide configured to form a complex with the endonuclease. In some embodiments, the engineered nuclease system comprises an endonuclease comprising a sequence with at least 100% sequence identity to any one of SEQ ID NOs: 25-198, 221-459, 489-580, 617-668, and 674-675 and an engineered guide polynucleotide configured to form a complex with the endonuclease.

In some embodiments, the engineered nuclease system comprises an endonuclease comprising a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 70% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 75% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 75% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 80% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 81% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 82% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 83% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 84% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 85% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 86% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 87% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 88% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 89% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 90% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 91% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 92% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 93% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 94% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 95% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 96% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 97% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 98% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 99% sequence identity to any one of SEQ ID NOs: 581-612, 989-1002, 1260-1273, 1322-1324, and 1329-1347 and an engineered guide polynucleotide configured to form a complex with the endonuclease. In some embodiments, the engineered nuclease system comprises an endonuclease comprising a sequence with at least 100% sequence identity to any one of SEQ ID NOs: 581-612, 989-1002, 1260-1273, 1322-1324, and 1329-1347 and an engineered guide polynucleotide configured to form a complex with the endonuclease.

In some embodiments, the engineered nuclease system comprises an endonuclease comprising a sequence with at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 70% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 75% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 75% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 80% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 81% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 82% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 83% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 84% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 85% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 86% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 87% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 88% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 89% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 90% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 91% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 92% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 93% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 94% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 95% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 96% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 97% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 98% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 99% sequence identity to any one of SEQ ID NOs: 976-979 and 1274-1288 and an engineered guide polynucleotide configured to form a complex with the endonuclease. In some embodiments, the engineered nuclease system comprises an endonuclease comprising a sequence with at least 100% sequence identity to any one of SEQ ID NOs: 976-979 and 1274-1288 and an engineered guide polynucleotide configured to form a complex with the endonuclease.

In some embodiments, the engineered guide polynucleotide is a single guide nucleic acid. In some embodiments, the engineered guide polynucleotide is a dual guide nucleic acid. In some embodiments, the engineered guide polynucleotide is RNA. In some embodiments, the endonuclease binds non-covalently to the engineered guide polynucleotide. In some embodiments, the endonuclease is covalently linked to the engineered guide polynucleotide.

In some embodiments, the engineered nuclease system comprises an endonuclease comprising a sequence having at least about 70% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising a sequence having at least about 70% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391. In some embodiments, the engineered nuclease system comprises an endonuclease comprising a sequence having at least about 75% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising a sequence having at least about 75% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391. In some embodiments, the engineered nuclease system comprises an endonuclease comprising a sequence having at least about 80% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising a sequence having at least about 80% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391. In some embodiments, the engineered nuclease system comprises an endonuclease comprising a sequence having at least about 85% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising a sequence having at least about 85% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391. In some embodiments, the engineered nuclease system comprises an endonuclease comprising a sequence having at least about 90% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising a sequence having at least about 90% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391. In some embodiments, the engineered nuclease system comprises an endonuclease comprising a sequence having at least about 95% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising a sequence having at least about 95% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391. In some embodiments, the engineered nuclease system comprises an endonuclease comprising a sequence having at least about 96% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising a sequence having at least about 96% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391. In some embodiments, the engineered nuclease system comprises an endonuclease comprising a sequence having at least about 97% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising a sequence having at least about 97% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391. In some embodiments, the engineered nuclease system comprises an endonuclease comprising a sequence having at least about 98% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising a sequence having at least about 98% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391. In some embodiments, the engineered nuclease system comprises an endonuclease comprising a sequence having at least about 99% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising a sequence having at least about 99% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391. In some embodiments, the engineered nuclease system comprises an endonuclease comprising 100% identity to any one of SEQ ID NOs: 1-198, 221-459, 463-612, 617-668, 674-675, 975-1002, 1322-1324, 1329-1347, 1350-1368, and 1415-1440 and an engineered guide polynucleotide configured to form a complex with the endonuclease, the engineered guide polynucleotide comprising 100% identity to any one of SEQ ID NOs: 199-203, 460-461, 613-616, 669-670, 672-673, 677-974, 1003-1022, 1231-1259, 1327-1328, 1348, 1369-1372, 1392-1414, and 1376-1391.

In some embodiments, the engineered nuclease system comprises an endonuclease comprising a sequence having at least about 70% identity to any one of SEQ ID NOs: 1, 463-486, 981-988, and 1289-1312 and an engineered guide polynucleotide configured to form a complex with the endonuclease, the engineered guide polynucleotide comprising a sequence having at least about 70% identity to any one of SEQ ID NOs: 199, 201, 669-670, and 1003-1005. In some embodiments, the engineered nuclease system comprises an endonuclease comprising a sequence having at least about 75% identity to any one of SEQ ID NOs: 1, 463-486, 981-988, and 1289-1312 and an engineered guide polynucleotide configured to form a complex with the endonuclease, the engineered guide polynucleotide comprising a sequence having at least about 75% identity to any one of SEQ ID NOs: 199, 201, 669-670, and 1003-1005. In some embodiments, the engineered nuclease system comprises an endonuclease comprising a sequence having at least about 80% identity to any one of SEQ ID NOs: 1, 463-486, 981-988, and 1289-1312 and an engineered guide polynucleotide configured to form a complex with the endonuclease, the engineered guide polynucleotide comprising a sequence having at least about 80% identity to any one of SEQ ID NOs: 199, 201, 669-670, and 1003-1005. In some embodiments, the engineered nuclease system comprises an endonuclease comprising a sequence having at least about 85% identity to any one of SEQ ID NOs: 1, 463-486, 981-988, and 1289-1312 and an engineered guide polynucleotide configured to form a complex with the endonuclease, the engineered guide polynucleotide comprising a sequence having at least about 85% identity to any one of SEQ ID NOs: 199, 201, 669-670, and 1003-1005. In some embodiments, the engineered nuclease system comprises an endonuclease comprising a sequence having at least about 90% identity to any one of SEQ ID NOs: 1, 463-486, 981-988, and 1289-1312 and an engineered guide polynucleotide configured to form a complex with the endonuclease, the engineered guide polynucleotide comprising a sequence having at least about 90% identity to any one of SEQ ID NOs: 199, 201, 669-670, and 1003-1005. In some embodiments, the engineered nuclease system comprises an endonuclease comprising a sequence having at least about 95% identity to any one of SEQ ID NOs: 1, 463-486, 981-988, and 1289-1312 and an engineered guide polynucleotide configured to form a complex with the endonuclease, the engineered guide polynucleotide comprising a sequence having at least about 95% identity to any one of SEQ ID NOs: 199, 201, 669-670, and 1003-1005. In some embodiments, the engineered nuclease system comprises an endonuclease comprising a sequence having at least about 96% identity to any one of SEQ ID NOs: 1, 463-486, 981-988, and 1289-1312 and an engineered guide polynucleotide configured to form a complex with the endonuclease, the engineered guide polynucleotide comprising a sequence having at least about 96% identity to any one of SEQ ID NOs: 199, 201, 669-670, and 1003-1005. In some embodiments, the engineered nuclease system comprises an endonuclease comprising a sequence having at least about 97% identity to any one of SEQ ID NOs: 1, 463-486, 981-988, and 1289-1312 and an engineered guide polynucleotide configured to form a complex with the endonuclease, the engineered guide polynucleotide comprising a sequence having at least about 97% identity to any one of SEQ ID NOs: 199, 201, 669-670, and 1003-1005. In some embodiments, the engineered nuclease system comprises an endonuclease comprising a sequence having at least about 98% identity to any one of SEQ ID NOs: 1, 463-486, 981-988, and 1289-1312 and an engineered guide polynucleotide configured to form a complex with the endonuclease, the engineered guide polynucleotide comprising a sequence having at least about 98% identity to any one of SEQ ID NOs: 199, 201, 669-670, and 1003-1005. In some embodiments, the engineered nuclease system comprises an endonuclease comprising a sequence having at least about 99% identity to any one of SEQ ID NOs: 1, 463-486, 981-988, and 1289-1312 and an engineered guide polynucleotide configured to form a complex with the endonuclease, the engineered guide polynucleotide comprising a sequence having at least about 99% identity to any one of SEQ ID NOs: 199, 201, 669-670, and 1003-1005. In some embodiments, the engineered nuclease system comprises an endonuclease comprising 100% identity to any one of SEQ ID NOs: 1, 463-486, 981-988, and 1289-1312 and an engineered guide polynucleotide configured to form a complex with the endonuclease, the engineered guide polynucleotide comprising 100% identity to any one of SEQ ID NOs: 199, 201, 669-670, and 1003-1005.

In some embodiments, the engineered nuclease system comprises an endonuclease comprising a sequence having at least about 70% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising a sequence having at least about 70% identity to any one of SEQ ID NOs: 200, 202, 203, 613-616, 1348, 1369, and 1392-1414. In some embodiments, the engineered nuclease system comprises an endonuclease comprising a sequence having at least about 75% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising a sequence having at least about 75% identity to any one of SEQ ID NOs: 200, 202, 203, 613-616, 1348, 1369, and 1392-1414. In some embodiments, the engineered nuclease system comprises an endonuclease comprising a sequence having at least about 80% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising a sequence having at least about 80% identity to any one of SEQ ID NOs: 200, 202, 203, 613-616, 1348, 1369, and 1392-1414. In some embodiments, the engineered nuclease system comprises an endonuclease comprising a sequence having at least about 85% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising a sequence having at least about 85% identity to any one of SEQ ID NOs: 200, 202, 203, 613-616, 1348, 1369, and 1392-1414. In some embodiments, the engineered nuclease system comprises an endonuclease comprising a sequence having at least about 90% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising a sequence having at least about 90% identity to any one of SEQ ID NOs: 200, 202, 203, 613-616, 1348, 1369, and 1392-1414. In some embodiments, the engineered nuclease system comprises an endonuclease comprising a sequence having at least about 95% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising a sequence having at least about 95% identity to any one of SEQ ID NOs: 200, 202, 203, 613-616, 1348, 1369, and 1392-1414. In some embodiments, the engineered nuclease system comprises an endonuclease comprising a sequence having at least about 96% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising a sequence having at least about 96% identity to any one of SEQ ID NOs: 200, 202, 203, 613-616, 1348, 1369, and 1392-1414. In some embodiments, the engineered nuclease system comprises an endonuclease comprising a sequence having at least about 97% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising a sequence having at least about 97% identity to any one of SEQ ID NOs: 200, 202, 203, 613-616, 1348, 1369, and 1392-1414. In some embodiments, the engineered nuclease system comprises an endonuclease comprising a sequence having at least about 98% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising a sequence having at least about 98% identity to any one of SEQ ID NOs: 200, 202, 203, 613-616, 1348, 1369, and 1392-1414. In some embodiments, the engineered nuclease system comprises an endonuclease comprising a sequence having at least about 99% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising a sequence having at least about 99% identity to any one of SEQ ID NOs: 200, 202, 203, 613-616, 1348, 1369, and 1392-1414. In some embodiments, the engineered nuclease system comprises an endonuclease comprising 100% identity to any one of SEQ ID NOs: 2-24, 487-488, 1313-1321, 1347, 1350-1368, and 1415-1440 and an engineered guide polynucleotide configured to form a complex with the endonuclease, the engineered guide polynucleotide comprising 100% identity to any one of SEQ ID NOs: 200, 202, 203, 613-616, 1348, 1369, and 1392-1414.

In some embodiments, the engineered nuclease system comprises an endonuclease comprising a sequence having at least about 70% identity to any one of SEQ ID NOs: 25-198, 221-459, 489-580, 617-668, and 674-675 and an engineered guide polynucleotide configured to form a complex with the endonuclease, the engineered guide polynucleotide comprising a sequence having at least about 70% identity to any one of SEQ ID NOs: 460-461, 677-974, 1006-1012, and 1231-1259. In some embodiments, the engineered nuclease system comprises an endonuclease comprising a sequence having at least about 75% identity to any one of SEQ ID NOs: 25-198, 221-459, 489-580, 617-668, and 674-675 and an engineered guide polynucleotide configured to form a complex with the endonuclease, the engineered guide polynucleotide comprising a sequence having at least about 75% identity to any one of SEQ ID NOs: 460-461, 677-974, 1006-1012, and 1231-1259. In some embodiments, the engineered nuclease system comprises an endonuclease comprising a sequence having at least about 80% identity to any one of SEQ ID NOs: 25-198, 221-459, 489-580, 617-668, and 674-675 and an engineered guide polynucleotide configured to form a complex with the endonuclease, the engineered guide polynucleotide comprising a sequence having at least about 80% identity to any one of SEQ ID NOs: 460-461, 677-974, 1006-1012, and 1231-1259. In some embodiments, the engineered nuclease system comprises an endonuclease comprising a sequence having at least about 85% identity to any one of SEQ ID NOs: 25-198, 221-459, 489-580, 617-668, and 674-675 and an engineered guide polynucleotide configured to form a complex with the endonuclease, the engineered guide polynucleotide comprising a sequence having at least about 85% identity to any one of SEQ ID NOs: 460-461, 677-974, 1006-1012, and 1231-1259. In some embodiments, the engineered nuclease system comprises an endonuclease comprising a sequence having at least about 90% identity to any one of SEQ ID NOs: 25-198, 221-459, 489-580, 617-668, and 674-675 and an engineered guide polynucleotide configured to form a complex with the endonuclease, the engineered guide polynucleotide comprising a sequence having at least about 90% identity to any one of SEQ ID NOs: 460-461, 677-974, 1006-1012, and 1231-1259. In some embodiments, the engineered nuclease system comprises an endonuclease comprising a sequence having at least about 95% identity to any one of SEQ ID NOs: 25-198, 221-459, 489-580, 617-668, and 674-675 and an engineered guide polynucleotide configured to form a complex with the endonuclease, the engineered guide polynucleotide comprising a sequence having at least about 95% identity to any one of SEQ ID NOs: 460-461, 677-974, 1006-1012, and 1231-1259. In some embodiments, the engineered nuclease system comprises an endonuclease comprising a sequence having at least about 96% identity to any one of SEQ ID NOs: 25-198, 221-459, 489-580, 617-668, and 674-675 and an engineered guide polynucleotide configured to form a complex with the endonuclease, the engineered guide polynucleotide comprising a sequence having at least about 96% identity to any one of SEQ ID NOs: 460-461, 677-974, 1006-1012, and 1231-1259. In some embodiments, the engineered nuclease system comprises an endonuclease comprising a sequence having at least about 97% identity to any one of SEQ ID NOs: 25-198, 221-459, 489-580, 617-668, and 674-675 and an engineered guide polynucleotide configured to form a complex with the endonuclease, the engineered guide polynucleotide comprising a sequence having at least about 97% identity to any one of SEQ ID NOs: 460-461, 677-974, 1006-1012, and 1231-1259. In some embodiments, the engineered nuclease system comprises an endonuclease comprising a sequence having at least about 98% identity to any one of SEQ ID NOs: 25-198, 221-459, 489-580, 617-668, and 674-675 and an engineered guide polynucleotide configured to form a complex with the endonuclease, the engineered guide polynucleotide comprising a sequence having at least about 98% identity to any one of SEQ ID NOs: 460-461, 677-974, 1006-1012, and 1231-1259. In some embodiments, the engineered nuclease system comprises an endonuclease comprising a sequence having at least about 99% identity to any one of SEQ ID NOs: 25-198, 221-459, 489-580, 617-668, and 674-675 and an engineered guide polynucleotide configured to form a complex with the endonuclease, the engineered guide polynucleotide comprising a sequence having at least about 99% identity to any one of SEQ ID NOs: 460-461, 677-974, 1006-1012, and 1231-1259. In some embodiments, the engineered nuclease system comprises an endonuclease comprising 100% identity to any one of SEQ ID NOs: 25-198, 221-459, 489-580, 617-668, and 674-675 and an engineered guide polynucleotide configured to form a complex with the endonuclease, the engineered guide polynucleotide comprising 100% identity to any one of SEQ ID NOs: 460-461, 677-974, 1006-1012, and 1231-1259.

In some embodiments, the engineered nuclease system comprises an endonuclease comprising a sequence having at least about 70% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising a sequence having at least about 70% identity to any one of SEQ ID NOs: 672-673, 1013-1022, 1327-1328, 1370-1372, and 1376-1391. In some embodiments, the engineered nuclease system comprises an endonuclease comprising a sequence having at least about 75% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising a sequence having at least about 75% identity to any one of SEQ ID NOs: 672-673, 1013-1022, 1327-1328, 1370-1372, and 1376-1391. In some embodiments, the engineered nuclease system comprises an endonuclease comprising a sequence having at least about 80% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising a sequence having at least about 80% identity to any one of SEQ ID NOs: 672-673, 1013-1022, 1327-1328, 1370-1372, and 1376-1391. In some embodiments, the engineered nuclease system comprises an endonuclease comprising a sequence having at least about 85% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising a sequence having at least about 85% identity to any one of SEQ ID NOs: 672-673, 1013-1022, 1327-1328, 1370-1372, and 1376-1391. In some embodiments, the engineered nuclease system comprises an endonuclease comprising a sequence having at least about 90% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising a sequence having at least about 90% identity to any one of SEQ ID NOs: 672-673, 1013-1022, 1327-1328, 1370-1372, and 1376-1391. In some embodiments, the engineered nuclease system comprises an endonuclease comprising a sequence having at least about 95% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising a sequence having at least about 95% identity to any one of SEQ ID NOs: 672-673, 1013-1022, 1327-1328, 1370-1372, and 1376-1391. In some embodiments, the engineered nuclease system comprises an endonuclease comprising a sequence having at least about 96% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising a sequence having at least about 96% identity to any one of SEQ ID NOs: 672-673, 1013-1022, 1327-1328, 1370-1372, and 1376-1391. In some embodiments, the engineered nuclease system comprises an endonuclease comprising a sequence having at least about 97% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising a sequence having at least about 97% identity to any one of SEQ ID NOs: 672-673, 1013-1022, 1327-1328, 1370-1372, and 1376-1391. In some embodiments, the engineered nuclease system comprises an endonuclease comprising a sequence having at least about 98% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising a sequence having at least about 98% identity to any one of SEQ ID NOs: 672-673, 1013-1022, 1327-1328, 1370-1372, and 1376-1391. In some embodiments, the engineered nuclease system comprises an endonuclease comprising a sequence having at least about 99% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising a sequence having at least about 99% identity to any one of SEQ ID NOs: 672-673, 1013-1022, 1327-1328, 1370-1372, and 1376-1391. In some embodiments, the engineered nuclease system comprises an endonuclease comprising 100% identity to any one of SEQ ID NOs: 581-612, 989-1002, 1260-1273, 1322-1324, and 1329-1347 and an engineered guide polynucleotide configured to form a complex with the endonuclease, the engineered guide polynucleotide comprising 100% identity to any one of SEQ ID NOs: 672-673, 1013-1022, 1327-1328, 1370-1372, and 1376-1391.

In some embodiments, the engineered guide polynucleotide comprises a sequence having at least 90% sequence identity to any one of SEQ ID NOs: 1327-1328, 1348, 1369-1372, 1376-1391, 1392-1414, and 1470-2242. In some embodiments, the engineered guide polynucleotide comprises a sequence having at least 92% sequence identity to any one of SEQ ID NOs: 1327-1328, 1348, 1369-1372, 1376-1391, 1392-1414, and 1470-2242. In some embodiments, the engineered guide polynucleotide comprises a sequence having at least 93% sequence identity to any one of SEQ ID NOs: 1327-1328, 1348, 1369-1372, 1376-1391, 1392-1414, and 1470-2242. In some embodiments, the engineered guide polynucleotide comprises a sequence having at least 94% sequence identity to any one of SEQ ID NOs: 1327-1328, 1348, 1369-1372, 1376-1391, 1392-1414, and 1470-2242. In some embodiments, the engineered guide polynucleotide comprises a sequence having at least 95% sequence identity to any one of SEQ ID NOs: 1327-1328, 1348, 1369-1372, 1376-1391, 1392-1414, and 1470-2242. In some embodiments, the engineered guide polynucleotide comprises a sequence having at least 96% sequence identity to any one of SEQ ID NOs: 1327-1328, 1348, 1369-1372, 1376-1391, 1392-1414, and 1470-2242. In some embodiments, the engineered guide polynucleotide comprises a sequence having at least 97% sequence identity to any one of SEQ ID NOs: 1327-1328, 1348, 1369-1372, 1376-1391, 1392-1414, and 1470-2242. In some embodiments, the engineered guide polynucleotide comprises a sequence having at least 98% sequence identity to any one of SEQ ID NOs: 1327-1328, 1348, 1369-1372, 1376-1391, 1392-1414, and 1470-2242. In some embodiments, the engineered guide polynucleotide comprises a sequence having at least 99% sequence identity to any one of SEQ ID NOs: 1327-1328, 1348, 1369-1372, 1376-1391, 1392-1414, and 1470-2242. In some embodiments, the engineered guide polynucleotide comprises a sequence having 100% sequence identity to any one of SEQ ID NOs: 1327-1328, 1348, 1369-1372, 1376-1391, 1392-1414, and 1470-2242.

In some embodiments, the engineered nuclease system further comprises a single-stranded DNA repair template. In some embodiments, the engineered nuclease system further comprises a double-stranded DNA repair template. In some embodiments, the single- or double-stranded DNA repair template comprises from 5′ to 3′ a first homology arm comprising a sequence of at least 20 nucleotides 5′ to the target deoxyribonucleic acid sequence. In some embodiments, the single- or double-stranded DNA repair template comprises from 5′ to 3′ a synthetic DNA sequence of at least 10 nucleotides. In some embodiments, the single- or double-stranded DNA repair template comprises from 5′ to 3′ a second homology arm comprising a sequence of at least 20 nucleotides 3′ to the target sequence. In some embodiments, the single- or double-stranded DNA repair template comprises from 5′ to 3′: a first homology arm comprising a sequence of at least 20 nucleotides 5′ to the target deoxyribonucleic acid sequence, a synthetic DNA sequence of at least 10 nucleotides, or a second homology arm comprising a sequence of at least 20 nucleotides 3′ to the target sequence.

In some embodiments, the first homology arm comprises a sequence of at least 10, at least 20, at least 30, at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides. In some embodiments, the first homology arm comprises a sequence of at least 10 nucleotides. In some embodiments, the first homology arm comprises a sequence of at least 20 nucleotides. In some embodiments, the first homology arm comprises a sequence of at least 30 nucleotides. In some embodiments, the first homology arm comprises a sequence of at least 40 nucleotides. In some embodiments, the first homology arm comprises a sequence of at least 50 nucleotides. In some embodiments, the first homology arm comprises a sequence of at least 60 nucleotides. In some embodiments, the first homology arm comprises a sequence of at least 70 nucleotides. In some embodiments, the first homology arm comprises a sequence of at least 80 nucleotides. In some embodiments, the first homology arm comprises a sequence of at least 90 nucleotides. In some embodiments, the first homology arm comprises a sequence of at least 100 nucleotides. In some embodiments, the first homology arm comprises a sequence of at least 110 nucleotides. In some embodiments, the first homology arm comprises a sequence of at least 120 nucleotides. In some embodiments, the first homology arm comprises a sequence of at least 130 nucleotides. In some embodiments, the first homology arm comprises a sequence of at least 140 nucleotides. In some embodiments, the first homology arm comprises a sequence of at least 150 nucleotides. In some embodiments, the first homology arm comprises a sequence of at least 175 nucleotides. In some embodiments, the first homology arm comprises a sequence of at least 200 nucleotides. In some embodiments, the first homology arm comprises a sequence of at least 250 nucleotides. In some embodiments, the first homology arm comprises a sequence of at least 300 nucleotides. In some embodiments, the first homology arm comprises a sequence of at least 400 nucleotides. In some embodiments, the first homology arm comprises a sequence of at least 500 nucleotides. In some embodiments, the first homology arm comprises a sequence of at least 750 nucleotides. In some embodiments, the first homology arm comprises a sequence of at least 1000 nucleotides.

In some embodiments, the engineered nuclease system further comprises a source of Mg²⁺.

In some embodiments, the present disclosure provides an endonuclease system described herein configured to cause a chemical modification of a nucleotide base within or proximal to a target locus targeted by the endonuclease system. In this case, chemical modification of a nucleotide base refers to modification of the chemical moiety involved in base-pairing rather than modification of the sugar or phosphate portion of the nucleotide. In some embodiments, the chemical modification comprises deamination of an adenosine or a cytosine nucleotide. In some embodiments, endonuclease systems configured to cause a chemical modification comprises an endonuclease having a base editor coupled or fused in frame to said endonuclease. In some embodiments, the endonuclease to which the base editor is fused or coupled comprises a deactivating mutation in at least one catalytic residue of the endonuclease (e.g., in the RuvC domain). In some embodiments, the base editor is fused N- or C-terminally to said endonuclease or linked via chemical conjugation. In some embodiments, base editors include any adenosine or cytosine deaminases, comprising but not limited to Adenosine Deaminase RNA Specific 1 (ADAR1), Adenosine Deaminase RNA Specific 2 (ADAR2), Apolipoprotein B MRNA Editing Enzyme Catalytic Subunit 1 (APOBEC1), Apolipoprotein B MRNA Editing Enzyme Catalytic Subunit 2 (APOBEC2), Apolipoprotein B MRNA Editing Enzyme Catalytic Subunit 3A (APOBEC3A), Apolipoprotein B MRNA Editing Enzyme Catalytic Subunit 3B (APOBEC3B), Apolipoprotein B MRNA Editing Enzyme Catalytic Subunit 3C (APOBEC3C), Apolipoprotein B MRNA Editing Enzyme Catalytic Subunit 3D (APOBEC3D), Apolipoprotein B MRNA Editing Enzyme Catalytic Subunit 3F (APOBEC3F), Apolipoprotein B MRNA Editing Enzyme Catalytic Subunit 3G (APOBEC3G), Apolipoprotein B MRNA Editing Enzyme Catalytic Subunit 3H (APOBEC3H), or Apolipoprotein B MRNA Editing Enzyme Catalytic Subunit 4 (APOBEC4), or a functional fragment thereof. In some embodiments, the base editor comprises a yeast, eukaryotic, mammalian, or human base editor.

In some embodiments, the present disclosure provides an endonuclease system described herein configured to cause a chemical modification of histone within or proximal to a target locus targeted by the endonuclease system. In some embodiments, endonuclease systems configured to cause a chemical modification of a histone comprise an endonuclease having a histone editor coupled or fused in frame to said endonuclease. In some embodiments, the histone editor is coupled or fused N- or C-terminally to the endonuclease. In some embodiments, the chemical modification comprise methylation, acetylation, demethylation, or deacetylation. In some embodiments, the endonuclease to which the histone editor is fused or coupled comprises a deactivating mutation in at least one catalytic residue of the endonuclease (e.g., in the RuvC domain). In some embodiments, the histone editor comprises a histone methyltransferase (e.g., ASH1L, DOT1L, EHMT1, EHMT2, EZH1, EZH2, MLL, MLL2, MLL3, MLL4, MLL5, NSD1, PRDM2, SET, SETBP1, SETD1A, SETD1B, SETD2, SETD3, SETD4, SETD5, SETD6, SETD7, SETD8, SETD9, SETDB1, SETDB2, SETMAR, SMYD1, SMYD2, SMYD3, SMYD4, SMYD5, SUV39H1, SUV39H2, SUV420H1, or SUV420H2), a histone demethylase (e.g., the KDM1, KDM2, KDM3, KDM4, KDM5, or KDM6 families), a histone acetyltransferase (e.g., GNATs or HAT family acetyltransferases), or a histone deacetylase (e.g., HDAC1, HDAC2, HDAC 3, HDAC4, HDAC5, HDAC6, HDAC7, HDAC8, HDAC9, HDAC10, HDAC11, SIRT1, SIRT2, SIRT3, SIRT4, SIRT5, SIRT6, or SIRT7). In some embodiments, the histone editor comprises a yeast, eukaryotic, mammalian, or human histone editor.

### Delivery and Vectors

Disclosed herein, in some embodiments, are nucleic acid sequences encoding an engineered nuclease system comprising an endonuclease and an engineered guide polynucleotide or components of the engineered nuclease system.

In some embodiments, the nucleic acid encoding the endonuclease system or components thereof is a DNA, for example a linear DNA, a plasmid DNA, or a minicircle DNA. In some embodiments, the nucleic acid encoding the engineered nuclease system is an RNA, for example a mRNA.

In some embodiments, the nucleic acid encoding the endonuclease system or components thereof is delivered by a nucleic acid-based vector. In some embodiments, the nucleic acid-based vector is a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the nucleic acid-based vector is selected from the list consisting of pSF-CMV-NEO-NH2-PPT-3XFLAG, pSF-CMV-NEO-COOH-3XFLAG, pSF-CMV-PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEFla-mCherry-N1 vector, pEFla-tdTomato vector, pSF-CMV-FMDV-Hygro, pSF-CMV-PGK-Puro, pMCP-tag(m), pSF-CMV—PURO-NH2-CMYC, pSF-OXB20-BetaGal, pSF-OXB20-Fluc, pSF-OXB20, pSF-Tac, pRI 101-AN DNA, pCambia2301, pTYB21, pKLAC2, pAc5.1/V5-His A, and pDEST8.

In some embodiments, the nucleic acid-based vector comprises a promoter. In some embodiments, the promoter is selected from the group consisting of a mini promoter, an inducible promoter, a constitutive promoter, and derivatives thereof. In some embodiments, the promoter is selected from the group consisting of CMV, CBA, EFla, CAG, PGK, TRE, U6, UAS, T7, Sp6, lac, araBad, trp, Ptac, p5, p19, p40, Synapsin, CaMKII, GRK1, and derivatives thereof. In some embodiments the promoter is a U6 promoter. In some embodiments, the promoter is a CAG promoter.

In some embodiments, the nucleic acid-based vector is a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is or a retrovirus.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the virus is AAV1 or a derivative thereof. In some embodiments, the virus is AAV2 or a derivative thereof. In some embodiments, the virus is AAV3 or a derivative thereof. In some embodiments, the virus is AAV4 or a derivative thereof. In some embodiments, the virus is AAV5 or a derivative thereof. In some embodiments, the virus is AAV6 or a derivative thereof. In some embodiments, the virus is AAV7 or a derivative thereof. In some embodiments, the virus is AAV8 or a derivative thereof. In some embodiments, the virus is AAV9 or a derivative thereof. In some embodiments, the virus is AAV10 or a derivative thereof. In some embodiments, the virus is AAV11 or a derivative thereof. In some embodiments, the virus is AAV12 or a derivative thereof. In some embodiments, the virus is AAV13 or a derivative thereof. In some embodiments, the virus is AAV14 or a derivative thereof. In some embodiments, the virus is AAV15 or a derivative thereof. In some embodiments, the virus is AAV16 or a derivative thereof. In some embodiments, the virus is AAV-rh8 or a derivative thereof. In some embodiments, the virus is AAV-rh10 or a derivative thereof. In some embodiments, the virus is AAV-rh20 or a derivative thereof. In some embodiments, the virus is AAV-rh39 or a derivative thereof. In some embodiments, the virus is AAV-rh74 or a derivative thereof. In some embodiments, the virus is AAV-rhM4-1 or a derivative thereof. In some embodiments, the virus is AAV-hu37 or a derivative thereof. In some embodiments, the virus is AAV-Anc80 or a derivative thereof. In some embodiments, the virus is AAV-Anc80L65 or a derivative thereof. In some embodiments, the virus is AAV-7m8 or a derivative thereof. In some embodiments, the virus is AAV-PHP-B or a derivative thereof. In some embodiments, the virus is AAV-PHP-EB or a derivative thereof. In some embodiments, the virus is AAV-2.5 or a derivative thereof. In some embodiments, the virus is AAV-2tYF or a derivative thereof. In some embodiments, the virus is AAV-3B or a derivative thereof. In some embodiments, the virus is AAV-LK03 or a derivative thereof. In some embodiments, the virus is AAV-HSC1 or a derivative thereof. In some embodiments, the virus is AAV-HSC2 or a derivative thereof. In some embodiments, the virus is AAV-HSC3 or a derivative thereof. In some embodiments, the virus is AAV-HSC4 or a derivative thereof. In some embodiments, the virus is AAV-HSC5 or a derivative thereof. In some embodiments, the virus is AAV-HSC6 or a derivative thereof. In some embodiments, the virus is AAV-HSC7 or a derivative thereof. In some embodiments, the virus is AAV-HSC8 or a derivative thereof. In some embodiments, the virus is AAV-HSC9 or a derivative thereof. In some embodiments, the virus is AAV-HSC10 or a derivative thereof. In some embodiments, the virus is AAV-HSC11 or a derivative thereof. In some embodiments, the virus is AAV-HSC12 or a derivative thereof. In some embodiments, the virus is AAV-HSC13 or a derivative thereof. In some embodiments, the virus is AAV-HSC14 or a derivative thereof. In some embodiments, the virus is AAV-HSC15 or a derivative thereof. In some embodiments, the virus is AAV-TT or a derivative thereof. In some embodiments, the virus is AAV-DJ/8 or a derivative thereof. In some embodiments, the virus is AAV-Myo or a derivative thereof. In some embodiments, the virus is AAV-NP40 or a derivative thereof. In some embodiments, the virus is AAV-NP59 or a derivative thereof. In some embodiments, the virus is AAV-NP22 or a derivative thereof. In some embodiments, the virus is AAV-NP66 or a derivative thereof. In some embodiments, the virus is AAV-HSC16 or a derivative thereof.

In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.

In some embodiments, the nucleic acid encoding the engineered nuclease system or components thereof is delivered by a non-nucleic acid-based delivery system (e.g., a non-viral delivery system). In some embodiments, the non-viral delivery system is a liposome. In some embodiments, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the nucleic acid is comprised in a lipid nanoparticle (LNP).

In some embodiments, the engineered nuclease system or components thereof is introduced into the cell in any suitable way, either stably or transiently. In some embodiments, the engineered nuclease system or components thereof is transfected into the cell. In some embodiments, the cell is transduced or transfected with a nucleic acid construct that encodes the engineered nuclease system or components thereof. For example, a cell is transduced (e.g., with a virus encoding the engineered nuclease system or components thereof), or transfected (e.g., with a plasmid encoding the engineered nuclease system or components thereof) with a nucleic acid that encodes the engineered nuclease system or components thereof, or the translated the engineered nuclease system or components thereof. In some embodiments, the transduction is a stable or transient transduction. In some embodiments, cells expressing the engineered nuclease system or components thereof or containing the engineered nuclease system or components thereof are transduced or transfected with one or more gRNA molecules, for example, when the engineered nuclease system or components thereof comprises a CRISPR nuclease. In some embodiments, a plasmid expressing the engineered nuclease system or components thereof is introduced into cells through electroporation, transient (e.g., lipofection) and stable genome integration (e.g., piggybac) and viral transduction (for example lentivirus or AAV) or other methods known to those of skill in the art. In some embodiments, the gene editing system is introduced into the cell as one or more polypeptides. In some embodiments, delivery is achieved through the use of RNP complexes. Delivery methods to cells for polypeptides and/or RNPs are known in the art, for example by electroporation or by cell squeezing.

Exemplary methods of delivery of nucleic acids include lipofection, nucleofection, electroporation, stable genome integration (e.g., piggybac), microinjection, biolistics, virosomes, liposomes, immunoliposomes, polycation or lipid nucleic acid conjugates, naked DNA, artificial virions, and agent-enhanced uptake of DNA. Lipofection is described in e.g., U.S. Pat. Nos. 5,049,386; 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of WO 91/17424 and WO 91/16024. In some embodiments, the delivery is to cells (e.g., in vitro or ex vivo administration) or target tissues (e.g., in vivo administration). In some embodiments, the nucleic acid is comprised in a liposome or a nanoparticle that specifically targets a host cell.

Additional methods for the delivery of nucleic acids to cells are known to those skilled in the art. See, for example, US 2003/0087817.

In some embodiments, the present disclosure provides a cell comprising a vector or a nucleic acid described herein. In some embodiments, the cell expresses a gene editing system or parts thereof. In some embodiments, the cell is a human cell. In some embodiments, the cell is genome edited ex vivo. In some embodiments, the cell is genome edited in vivo.

### Cells

Described herein, in certain embodiments, is a cell comprising the systems or vectors described herein.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NS0), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SYSY cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, W138, HeLa, Saos, C2C12, L cell, HT1080, HepG2, Huh7, K562, a primary cell, or derivative thereof.

### METHODS OF USE

Systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g., via cleavage of reverse-transcribed viral RNA or an amplified DNA sequence encoding a disease-causing mutation); as deactivated enzymes in combination with a probe to target and detect a specific nucleotide sequence (e.g., sequence encoding antibiotic resistance int bacteria); to render viruses inactive or incapable of infecting host cells by targeting viral genomes; to add genes or amend metabolic pathways to engineer organisms to produce valuable small molecules, macromolecules, or secondary metabolites; to establish a gene drive element for evolutionary selection, and/or to detect cell perturbations by foreign small molecules and nucleotides as a biosensor.

Described herein, in some embodiments, are methods for binding, cleaving, marking, or modifying a double-stranded deoxyribonucleic acid polynucleotide

Described herein, in certain embodiments, are methods of modifying a target nucleic acid locus, said method comprising delivering to said target nucleic acid locus said engineered nuclease system described herein, wherein said endonuclease is configured to form a complex with said engineered guide ribonucleic acid structure, and wherein said complex is configured such that upon binding of said complex to said target nucleic acid locus, said complex modifies said target nucleic locus.

In some embodiments, the method comprises delivering to the target nucleic acid locus the engineered nuclease system described herein. In some embodiments, the endonuclease is configured to form a complex with the engineered guide ribonucleic acid structure. In some embodiments, the complex is configured such that upon binding of the complex to the target nucleic acid locus, the complex modifies the target nucleic locus. In some embodiments, modifying the target nucleic acid locus comprises binding, nicking, cleaving, or marking the target nucleic acid locus.

In some embodiments, the target nucleic acid locus comprises deoxyribonucleic acid (DNA) or ribonucleic acid (RNA). In some embodiments, the target nucleic acid comprises genomic eukaryotic DNA, viral DNA, or bacterial DNA. In some embodiments, the target nucleic acid comprises bacterial DNA. In some embodiments, the bacterial DNA is derived from a bacterial species different to a species from which the endonuclease was derived. In some embodiments, the target nucleic acid locus is in vitro. In some embodiments, the nucleic acid locus is within a cell. In some embodiments, the endonuclease and the engineered guide nucleic acid structure are provided encoded on separate nucleic acid molecules. In some embodiments, the cell is a prokaryotic cell, a bacterial cell, a eukaryotic cell, a fungal cell, a plant cell, an animal cell, a mammalian cell, a rodent cell, a primate cell, or a human cell. In some embodiments, the cell is derived from a species different to a species from which the endonuclease is derived.

Described herein, in some embodiments, are methods of disrupting a TRAC locus in a cell, comprising contacting to said cell a composition comprising: at least 80% sequence identity to any one of SEQ ID NOs: 1324, 1329-1346, 1350-1368, and 1415-1440; and an engineered guide polynucleotide that forms a complex with the endonuclease and hybridizes to a target nucleic acid sequence, wherein said engineered guide RNA is configured to hybridize to any one of SEQ ID NOs: 1079-1082, 1145-1166, and 1169-1170.

Described herein, in some embodiments, are methods of disrupting an AAVS1 locus in a cell, comprising contacting to said cell a composition comprising at least 80% sequence identity to any one of SEQ ID NOs: 1324, 1329-1346, 1350-1368, and 1415-1440, and an engineered guide RNA, wherein said engineered guide RNA is configured to form a complex with said endonuclease and said engineered guide RNA comprises a spacer sequence configured to hybridize to a region of said locus, wherein said engineered guide RNA is configured to hybridize to any one of SEQ ID NOs: 1105-1122 and 2301-2330.

In some embodiments, delivering the engineered nuclease system to the target nucleic acid locus comprises delivering the nucleic acid described herein or the vector described herein. In some embodiments, delivering the engineered nuclease system to the target nucleic acid locus comprises delivering a nucleic acid comprising an open reading frame encoding the endonuclease. In some embodiments, the nucleic acid comprises a promoter to which the open reading frame encoding the endonuclease is operably linked. In some embodiments, delivering the engineered nuclease system to the target nucleic acid locus comprises delivering a capped mRNA containing the open reading frame encoding said endonuclease. In some embodiments, delivering the engineered nuclease system to said target nucleic acid locus comprises delivering a translated polypeptide.

In some embodiments, delivering the engineered nuclease system to the target nucleic acid locus comprises delivering a deoxyribonucleic acid (DNA) encoding the engineered guide ribonucleic acid structure operably linked to a ribonucleic acid (RNA) pol III promoter. In some embodiments, the endonuclease induces a single-stranded break or a double-stranded break at or proximal to the target locus.

### Kits

In some embodiments, this disclosure provides kits comprising one or more nucleic acid constructs encoding the various components of the engineered nuclease system. In some embodiments, the nucleotide sequence comprises a heterologous promoter that drives expression of the engineered nuclease system components.

In some embodiments, the engineered nuclease system disclosed herein is assembled into a pharmaceutical, diagnostic, or research kit to facilitate its use in therapeutic, diagnostic, or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions, in some embodiments, are in a form prescribed by a governmental agency regulating the manufacture, use, or sale of pharmaceuticals or biological products, which instructions can also reflect approval by the agency of manufacture, use, or sale for animal administration.

### EXAMPLES

### Example 1—SMARTs Nucleases

### Bioinformatics Identification of SMART Nucleases

SMART nuclease homologs were discovered by mining a large assembly-driven metagenomic database of microbial, viral, and eukaryotic genomes. HMM profiles of previously described SMART nucleases were built and searched against all proteins in the database using a software package for sequence analysis. CRISPR arrays were predicted on assembled contigs with a program to find CRISPRs in full genomes or environmental datasets such as assembled contigs from metagenomes. Proteins were filtered by e-value (<1×10⁵) and size (>550 amino acids and <1000 amino acids), and partial ORFs were removed. Sequences were aligned with reference Type II nucleases, and SMART nucleases and a phylogenetic tree was built. The MG34 and MG102 clades were identified based on their location relative to previously identified SMART effectors, and novel candidates were selected for in vitro screening.

### Computational Reconstruction of Ancestral SMART Nuclease Sequences

To generate additional diversity of SMART nucleases, ancestral sequence reconstruction algorithms were used to generate nuclease sequences for the MG34 and MG102 families. For this analysis, 441 SMART I protein sequences were aligned with parameters L-INS-i G-INS-i and a phylogenetic tree was built. The trees were rooted using SpCas9 and SaCas9. Sequence reconstruction was performed. Insertions and deletions were identified manually for each reconstructed node.

In addition, MG34 ancestor variants were generated using two different methodologies. In the first method, previously-generated ancestors were modified by removing amino acid positions to approximate the sequence length of MG34-1 (SEQ ID NO: 2). In the second method, chimeras were designed from previously-generated ancestors by fusing the recognition lobe from active candidates with the protein backbone of active ancestors to determine if sequence divergence in the recognition lobe is at least partially responsible for the lack of activity of particular ancestral sequences.

### Bioinformatics Results

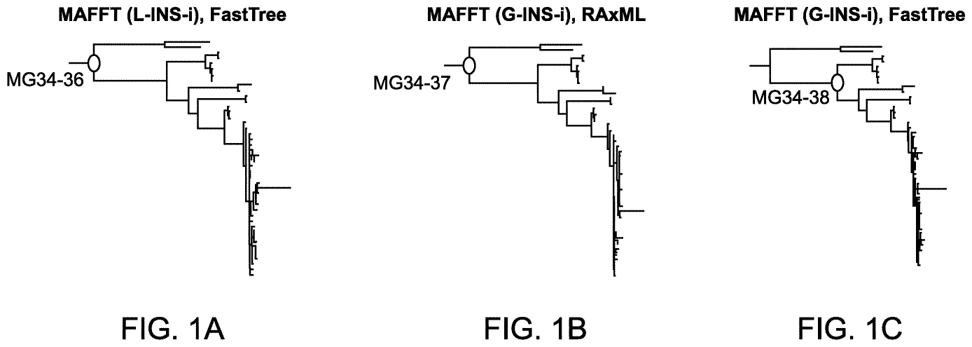

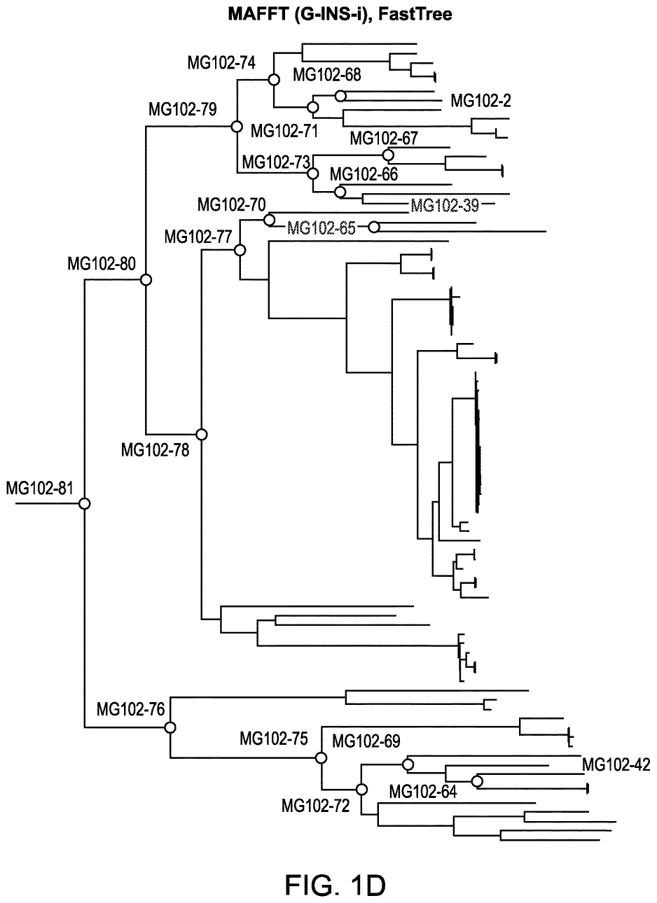

Based on mining of the metagenomics database, one novel MG34 sequence, MG34-35 (SEQ ID NO: 1347) and three novel MG102 sequences, MG102-63 (SEQ ID NO: 1324), MG102-82 (SEQ ID NO: 1322), and MG102-83 (SEQ ID NO: 1323) were identified. Three new ancestral sequences (Table 3) were generated for the MG34 clade using different alignment and tree-building algorithm combinations (FIGS. 1A-1C). Eighteen new ancestral sequences were generated for the MG102 clade (FIG. 1D; SEQ ID NOs: 1329-1347). Four MG34 ancestral variants (Table 4) were generated from previously-identified ancestral sequences by removing positions absent in the reference sequence MG34-1 (SEQ ID NO: 2). Twelve MG34 ancestral chimeras (Table 5) were generated by swapping in the recognition lobe of active candidates into other ancestral sequences.

TABLE 3

| Comparison of ancestral MG34 candidates to | | |
|---|---|---|
| the SMART I nuclease MG34-1. | | |
| | SEQ | MG34-1 |
| MG code | ID NO | % Identity |
| MG34-36 | 1350 | 68 |
| MG34-37 | 1351 | 68 |
| MG34-38 | 1351 | 79 |

TABLE 4
MG34 ancestor masked variants.

| MG | SEQ | MG34-1 | Original |
|---|---|---|---|
| code | ID NO | % Identity | sequence |
| MG34-39 | 1353 | 67 | MG34-26 (SEQ ID NO: 1313) |
| MG34-40 | 1354 | 72 | MG34-28 (SEQ ID NO: 1315) |
| MG34-41 | 1355 | 75 | MG34-30 (SEQ ID NO: 1317) |
| MG34-42 | 1356 | 88 | MG34-31 (SEQ ID NO: 1318) |

TABLE 5
MG34 ancestor chimeras

| | | | Recognition | | Backbone | MG34-1 |
|---|---|---|---|---|---|---|
| Chimera | | Recognition | lobe positions | Protein | positions | % |
| number | SEQ ID NO | Lobe | included | backbone | included | Identity |
| MG34- | 1357 | MG34-27 | 41-288 | MG34-36 | 1-40, 275- | 69 |
| 43 | | (SEQ ID NO: | | (SEQ ID NO: | 747 | |
| | | 1314) | | 1350) | | |
| MG34- | 1358 | MG34-38 | 41-274 | MG34-36 | 1-40, 275- | 72 |
| 44 | | (SEQ ID NO: | | (SEQ ID NO: | 747 | |
| | | 1352) | | 1350) | | |
| MG34- | 1359 | MG34-27 | 41-288 | MG34-37 | 1-40, 275- | 69 |
| 45 | | (SEQ ID No. | | (SEQ ID NO: | 747 | |
| | | 1314) | | 1351) | | |
| MG34- | 1360 | MG34-38 | 41-274 | MG34-37 | 1-40, 275- | 71 |
| 46 | | (SEQ ID NO: | | (SEQ ID NO: | 747 | |
| | | 1352) | | 1351) | | |
| MG34- | 1361 | MG34-27 | 41-288 | MG34-39 | 1-40, 275- | 69 |
| 47 | | (SEQ ID No. | | (SEQ ID NO: | 747 | |
| | | 1314) | | 1353) | | |
| MG34- | 1362 | MG34-38 | 41-274 | MG34-39 | 1-40, 275- | 72 |
| 48 | | (SEQ ID NO: | | (SEQ ID NO: | 745 | |
| | | 1352) | | 1353) | | |
| MG34- | 1363 | MG34-27 | 41-288 | MG34-40 | 1-40, 275- | 73 |
| 49 | | (SEQ ID NO: | | (SEQ ID NO: | 748 | |
| | | 1314) | | 1354) | | |
| MG34- | 1364 | MG34-38 | 41-274 | MG34-40 | 1-40, 275- | 76 |
| 50 | | (SEQ ID NO: | | (SEQ ID NO: | 748 | |
| | | 1352) | | 1354) | | |
| MG34- | 1365 | MG34-27 | 41-288 | MG34-41 | 1-40, 275- | 77 |
| 51 | | (SEQ ID NO: | | (SEQ ID NO: | 747 | |
| | | 1314) | | 1355) | | |
| MG34- | 1366 | MG34-38 | 41-274 | MG34-41 | 1-40, 275- | 80 |
| 52 | | (SEQ ID NO: | | (SEQ ID NO: | 747 | |
| | | 1352) | | 1355) | | |
| MG34- | 1367 | MG34-27 | 41-288 | MG34-42 | 1-40, 275- | 72 |
| 53 | | (SEQ ID No. | | (SEQ ID NO: | 745 | |
| | | 1314) | | 1356) | | |
| MG34- | 1368 | MG34-38 | 41-274 | MG34-42 | 1-40, 275- | 75 |
| 54 | | (SEQ ID NO: | | (SEQ ID NO: | 745 | |
| | | 1352) | | 1355) | | |

In vitro PAM Determination Assays

Candidate MG34 nuclease effector proteins were codon optimized for E. coli and cloned into a vector with a T7 promoter and C-terminal His tag. The gene was PCR amplified with primer binding sites 150 bp upstream and downstream from the T7 promoter and terminator sequences, respectively. This PCR product was added to a reconstituted protein synthesis system where all components needed for in vitro transcription and translation are purified from E. coli at 5 nM minimum final concentration and expressed for 2 hr at 37°CC. A cleavage reaction was assembled in 10 mM Tris pH 7.5, 100 mM NaCl, and 10 mM MgCl2 with a 5-fold dilution of the protein synthesis system, 5 nM of an 8N PAM plasmid library, and 50 nM of sgRNA targeting the PAM library. The sgRNA sequences used were some or all of those identified from four active MG34 homologs: MG34-1 (sg1) (SEQ ID NO: 613), MG34-9 (sg9) (SEQ ID NO: 615), MG34-16 (sg16) (SEQ ID NO: 616), and MG34-25 (sg25) (SEQ ID NO: 1369). The cleavage products from the protein synthesis system reactions were recovered via clean up with beads. The DNA was blunted via addition of Klenow fragments and dNTPs. Blunt-end products were ligated with a 100-fold excess of double stranded adapter sequences and used as template for the preparation of an NGS library, from which PAM requirements were determined from sequence analysis. Raw NGS reads were filtered by a quality score >20. The 14-24 bp representing the known DNA sequence from the backbone adjacent to the PAM was used as a reference to find the PAM-proximal region and the 8 bp adjacent were identified as the putative PAM. The distance between the PAM and the ligated adapter was also measured for each read. Reads that did not have an exact match to the reference sequence or adapter sequence were excluded. PAM sequences were filtered by cut site frequency such that only PAMs with the most frequent cut site ±2 bp were included in the analysis. The filtered list of PAM sequences was used to generate a sequence logo.

### Experimental Results

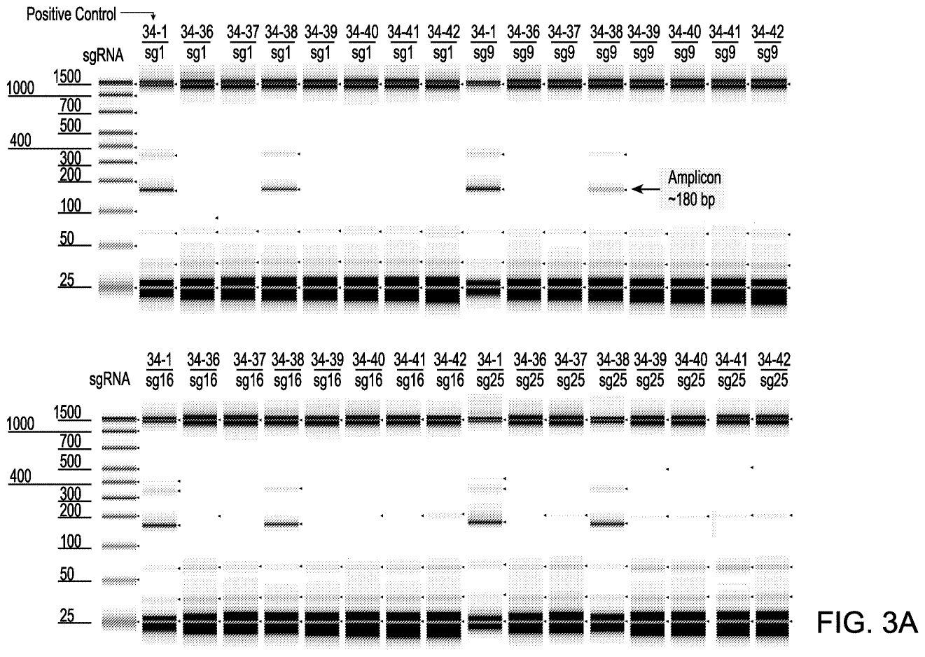

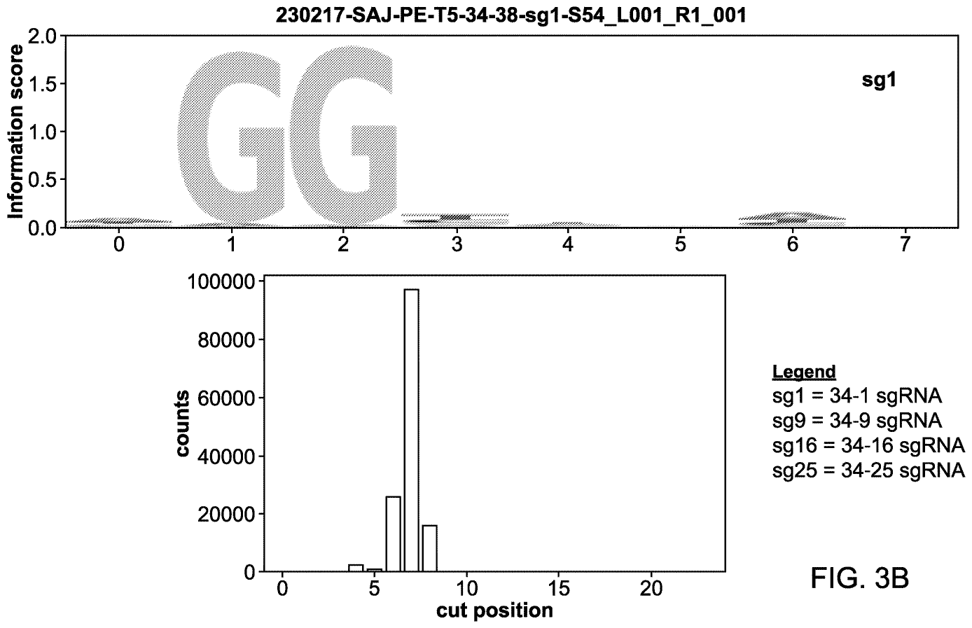

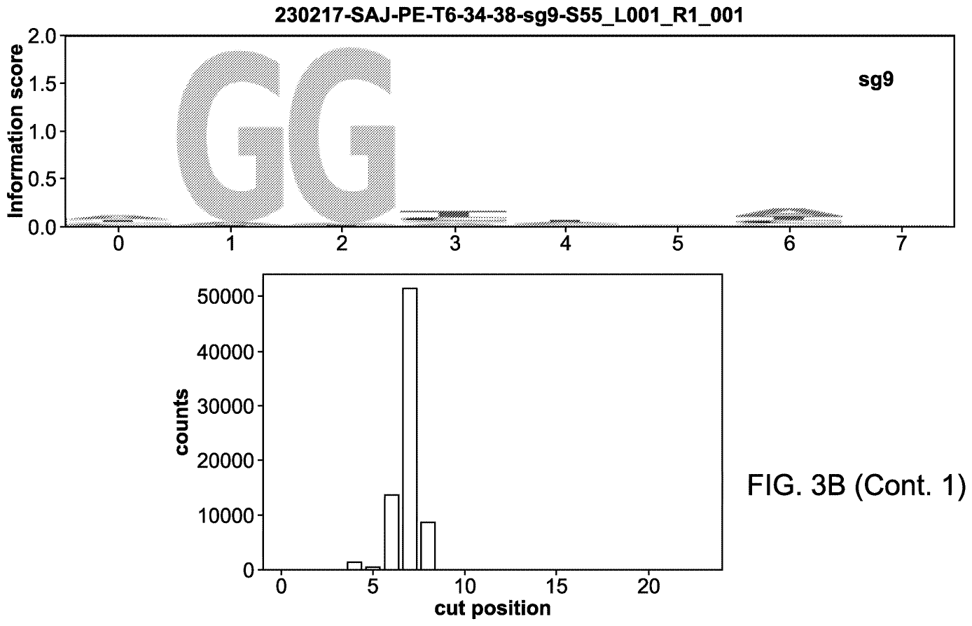

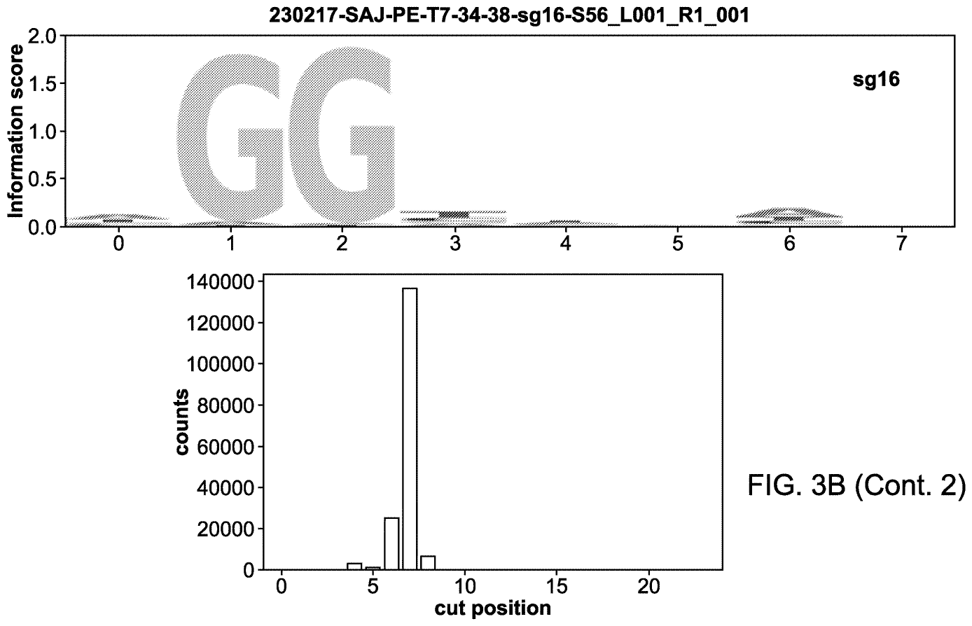

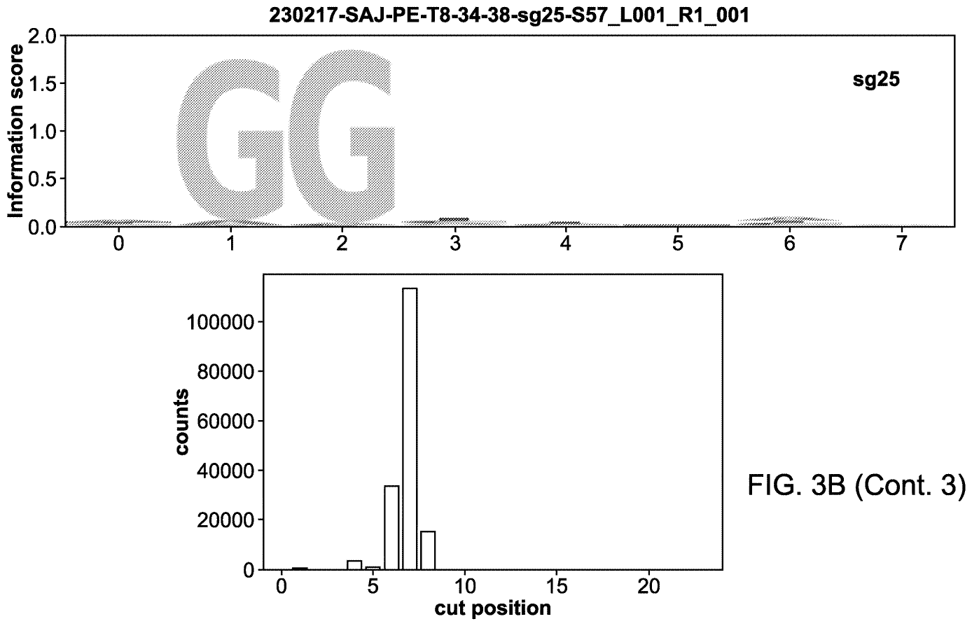

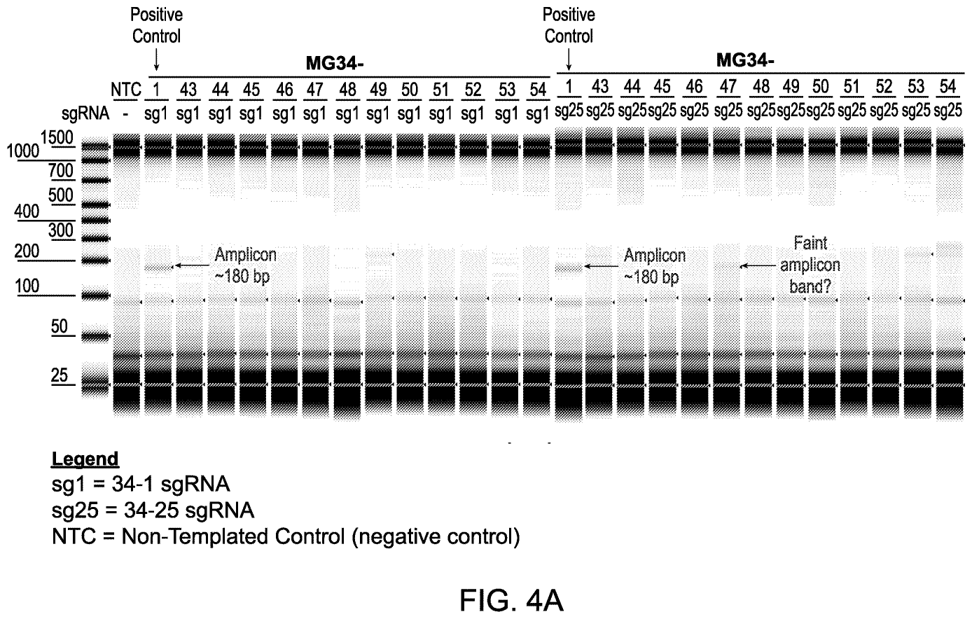

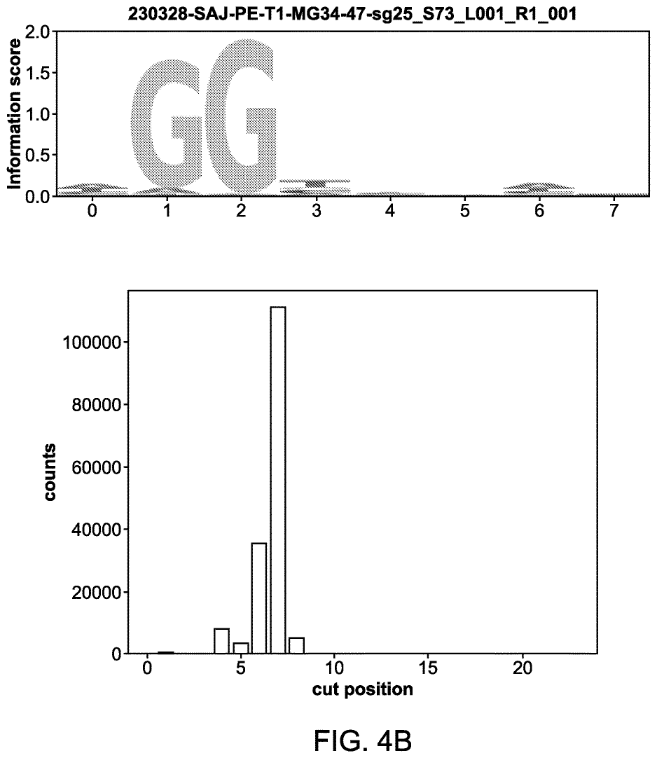

Three new MG34 nucleases were found to be active in vitro using the guide RNAs from active MG34 nucleases (FIGS. 2A-2B, 3A-3B, and 4A-4B): MG34-35 (SEQ ID NO: 1347) is a novel system identified from metagenomics data; MG34-38 (SEQ ID NO: 1352) is a reconstructed MG34 ancestor; and MG34-47 (SEQ ID NO: 1361) is a chimeric ancestor consisting of the recognition lobe of one ancestor inserted into the backbone of another ancestor (Table 5). These nucleases were active with all four tested sgRNAs, as shown by the expected cleavage band at approximately 180 bp (FIGS. 2A-2B, 3A-3B, and 4A-4B). The 3′ PAM sequence nGG appears to be the most commonly recognized by these nucleases, with subtle differences between them, and they exhibit a preference for cleavage at positions 6-8 from the PAM (FIGS. 2A-2B, 3A-3B, and 4A-4B).

### Example 2—SMART I Nucleases Recognize Diverse PAM Sequences and are Active in RNP Complexes

### In Vitro PAM Preference Assay

Novel SMART I effectors were quantitatively assayed for cleavage activity via an in vitro cleavage assay. The effectors were expressed in in vitro transcription/translation (IVTT) reactions from a PCR template as described in Example 1, either in the absence or presence of 0.4 μM of the single guide RNA from other active MG34 nucleases. After 2 hour expression, the IVTT mixture containing the RNP was incubated in a reaction consisting of 30% v/v IVTT and 5 nM of a plasmid DNA target with nRR PAMs (nAA, nAG, nGA, or nGG PAMs) in 10 mM Tris pH 7.5, 100 mM NaCl, and 10 mM MgCl2 for 1 hour. Cleavage products were analyzed by nucleic acid electrophoresis and peak area for uncleaved (~3500 bp supercoiled) and cleavage products (~2200 bp linearized) were plotted as a percent of RNA-guided cleavage.

### Results

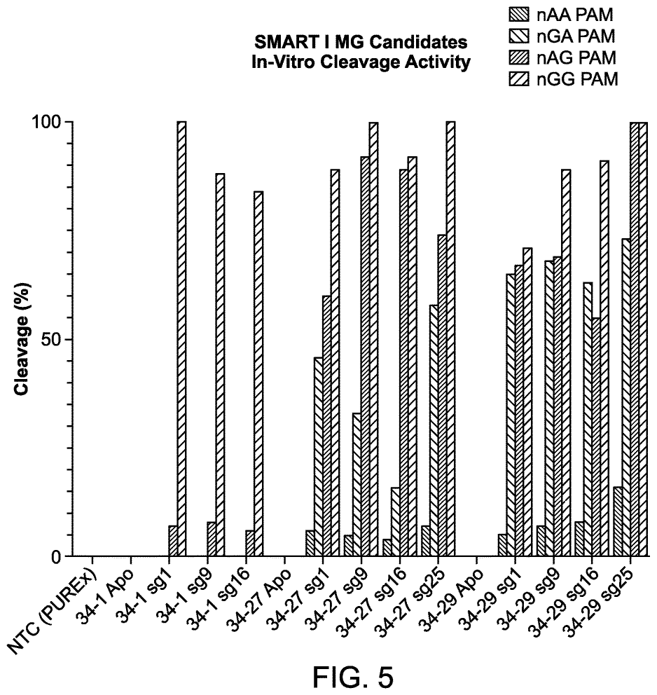

SMART I nucleases MG34-27 (SEQ ID NO: 1314) and MG34-29 (SEQ ID NO: 1316) were most active with targets with nGG and nAG PAM sequences, but also showed significant activity with nGA and more limited activity with nAA PAM sequences (Table 6; FIG. 5). The amount of cleavage of targets with each PAM sequence differs depending on the single guide RNA used. For example, MG34-27 shows the highest activity for the nGG PAM with sg25 (SEQ ID NO: 1369) but shows the highest activity for the nAG PAM with sg9 (SEQ ID NO: 615; FIG. 5).

TABLE 6

| Percent cleavage of individual PAM plasmid | | | | |
|---|---|---|---|---|
| targets with diverse single guide RNAs. | | | | |
| | nGA | nAA | nAG | nGG |
| | PAM | PAM | PAM | PAM |
| Sample | Target | Target | Target | Target |
| NTC | 0 | 0 | 0 | 0 |
| 34-1 Apo | 0 | 0 | 0 | 0 |
| 34-1 sg1 | 0 | 0 | 7 | 100 |
| 34-1 sg9 | N/A | N/A | 8 | 88 |
| 34-1 sg16 | N/A | N/A | 6 | 84 |
| 34-27 Apo | 0 | 0 | 0 | 0 |
| 34-27 sg1 | 6 | 46 | 60 | 89 |
| 34-27 sg9 | 5 | 33 | \|92 | 100 |
| 34-27 sg16 | 4 | 16 | 89 | 92 |
| 34-27 sg25 | 7 | 58 | 74 | 100 |
| 34-29 Apo | 0 | 0 | 0 | 0 |
| 34-29 sg1 | 5 | 65 | 67 | 71 |
| 34-29 sg9 | 7 | 68 | \|69 | 89 |
| 34-29 sg16 | 8 | 63 | \|55 | 91 |
| 34-29 sg25 | 16 | 73 | 100 | 100 |

### Nuclease Protein Purification and RNP Activity Assay

Candidate MG34 nucleases were cloned into pET21b under T7 RNA polymerase-controlled promoters and expressed in cells. Cultures were grown until induction at 37 °C and then temperature was lowered to 18 °C and the cells expressed for 18 hours. The lysis buffers contained 500 mM NaCl, 10% Glycerol, 0.5 mM TCEP and 50 mM Buffering agent. For MG34-27, CHES pH 9.0 was the buffering agent; for MG34-29 a range of buffers were tried—MES pH 6.0, Tris pH 7.5, CHES pH 9.0. Samples with 2× Laemmli buffer were separated on a Stain-Free 4-20% gradient SDS PAGE gel and visualized by fluorescence imaging.

To test the activity of purified RNP complexes, MG34-29 (50 nM) was complexed with 75 nM MG34-1 sgRNA (sg1) (SEQ ID NO: 613) and used to cleave a supercoiled plasmid containing an nGG PAM. MG34-27 (50 nM) was complexed with sgRNAs from MG34-1 (sg1) (SEQ ID NO: 613) and MG34-25 (sg25) (SEQ ID NO: 1369) and used to cleave four separate PAM containing plasmids (nAA, nAG, nGA, nGG).

### Purification and RNP Activity Assay Results

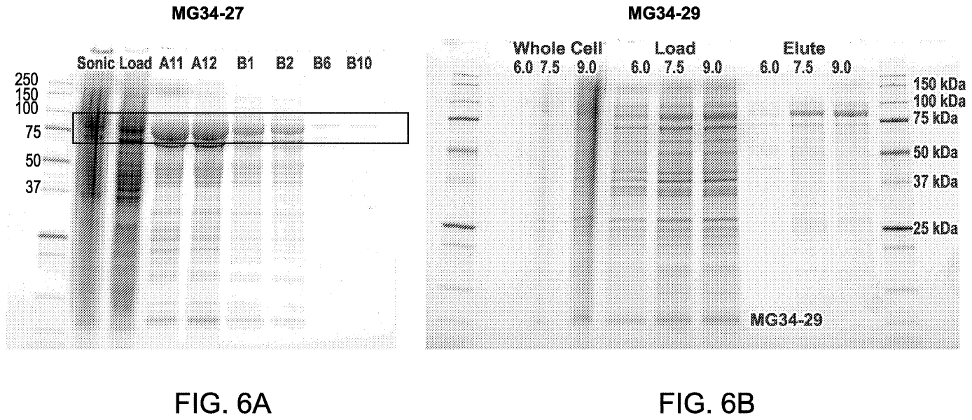

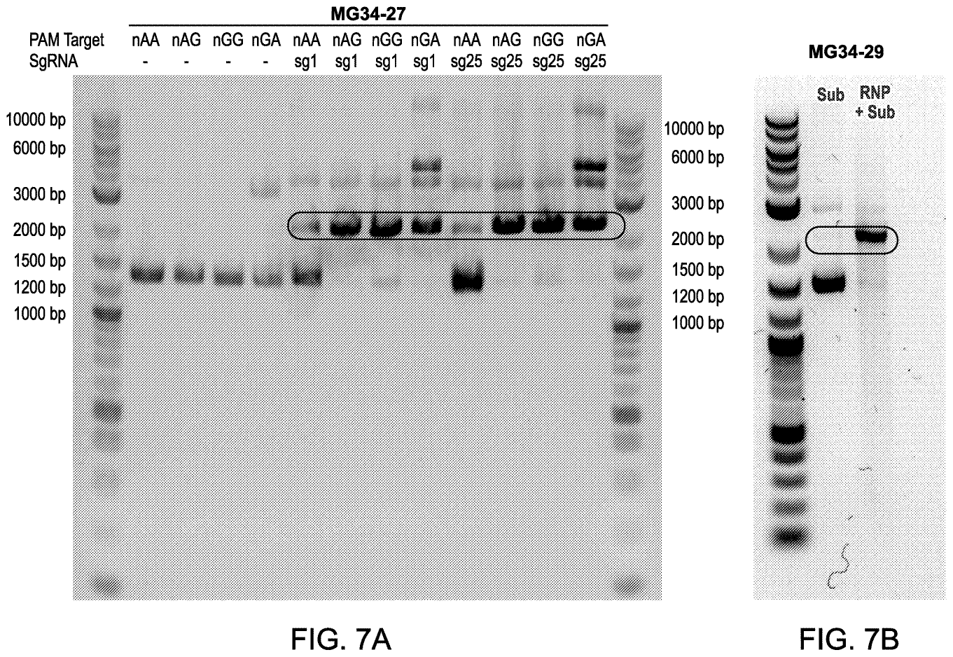

Initial purifications show that MG34-27 (SEQ ID NO: 1314) and MG34-29 (SEQ ID NO: 1316) are soluble in aqueous solution and readily purifiable at high yield (FIGS. 6A-6B). Activity assays demonstrate that these purified proteins exhibit active cleavage as RNP complexes with guide RNAs (FIGS. 7A-7B). MG34-29 efficiently cleaves a plasmid with an nGG PAM sequence (FIGS. 7A-7B) while MG34-27 efficiently cleaves plasmids with nAG, nGG, and nGA PAM sequences, with partial cleavage of plasmids with the nAA PAM sequence with both sg1 (SEQ ID NO: 613) and sg25 (SEQ ID NO: 1369; FIGS. 7A-7B).

### Example 3—Ancestral Reconstructions of SMART I Nucleases are Active Gene Editors in Human Cells

CRISPR-associated SMART I nucleases of the MG34 family are capable of efficient dsDNA cleavage activity in vitro and in E. coli, but they have not previously shown detectable levels of activity as nucleases in human cells. This family of enzymes has been expanded herein by applying ancestral sequence reconstruction methods for computational protein diversification. Computational reconstruction generated new MG34 effectors that were active for dsDNA cleavage in vitro. Herein, the activity of these enzymes in human cells was evaluated.

mRNA Production

Sequences for MG34-27 (SEQ ID NO: 1314) and MG34-29 (SEQ ID NO: 1316) were codon optimized for human expression and cloned into an expression vector with a T7 promoter, 5′ and 3′ UTRs, and a polyA tail. The coding sequence contained an N-terminal SV40 nuclear localization signal and a C-terminal nucleoplasmin nuclear localization signal. The expression vector was midi-prepped, linearized with SapI, and used for in vitro transcription with Hi-T7. In vitro transcription reactions contained N1-methylpseudouridine in place of uridine and transcription reagent. The resulting mRNA was checked for product size and purity and diluted to 250 ng/μL in sterile water for use in nucleofection.

### Nucleofection

K562 cells (ATCC #CCL-243) were cultured in IMDM media+10% FBS for 1-2 passages prior to nucleofection. On the day of nucleofection, cells were harvested, counted, washed in 1×PBS, and resuspended in buffer according to manufacturer instructions. 120,000 cells were distributed per well and nucleofected with 500 ng of mRNA and 200 pmol of sgRNA. For some experiments, the amount of guide added was varied from 100 to 400 pmol. Cells were added to recovery media and grown for 72 hours before genomic DNA was harvested. Resulting gDNA was diluted 1:3 and used as a template for NGS PCR. Indels were quantified from resulting NGS reads mapped to the reference amplicon.

### Results

MG34-27 and MG34-29 were each tested with 96 guides with NGG PAMs targeting the AAVS1 locus. The target sites are listed in Table 7.

TABLE 7
AAVS1 genome editing targets for MG34-27 and
MG34-29

| sgRNA | | sgRNA SEQ |
|---|---|---|
| well | Target site in genome | ID NO: |
| B7 | TTAGACCCAATATCAGGAGACT | 1400 |
| B11 | CCTGAAGTGGACATAGGGGCCC | 1401 |
| C7 | ACCCAATATCAGGAGACTAGGA | 1402 |
| C11 | AAGTGGACATAGGGGCCCGGGT | 1403 |
| D5 | GAGGTTCTGGCAAGGAGAGAGA | 1404 |
| D8 | CTGTCCCTAGTGGCCCCACTGT | 1405 |
| E6 | AAGGAATCTGCCTAACAGGAGG | 1406 |
| E7 | TATCAGGAGACTAGGAAGGAGG | 1407 |
| E8 | TGTCCCTAGTGGCCCCACTGTG | 1408 |
| E9 | ATTAACCGGCCCTGGGAATATA | 1409 |
| F7 | AGACTAGGAAGGAGGAGGCCTA | 1410 |
| G6 | GGAATCTGCCTAACAGGAGGTG | 1411 |
| G7 | TAGGAAGGAGGAGGCCTAAGGA | 1412 |
| H6 | GAATCTGCCTAACAGGAGGTGG | 1413 |
| H12 | AGCACAGACTAGAGAGGTAAGG | 1414 |

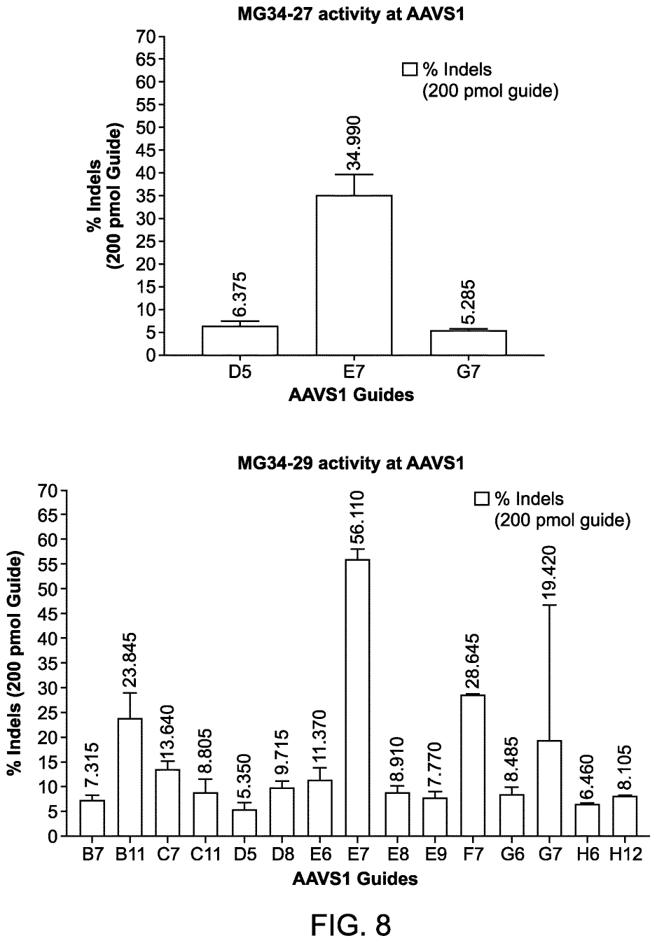

MG34-27 showed activity over 5% with three guides (FIG. 8, SEQ ID NOs: 1404, 1407, and 1412), while MG34-29 showed activity over 5% with 15 guides (FIG. 8, SEQ ID NOs: 1400-1414).

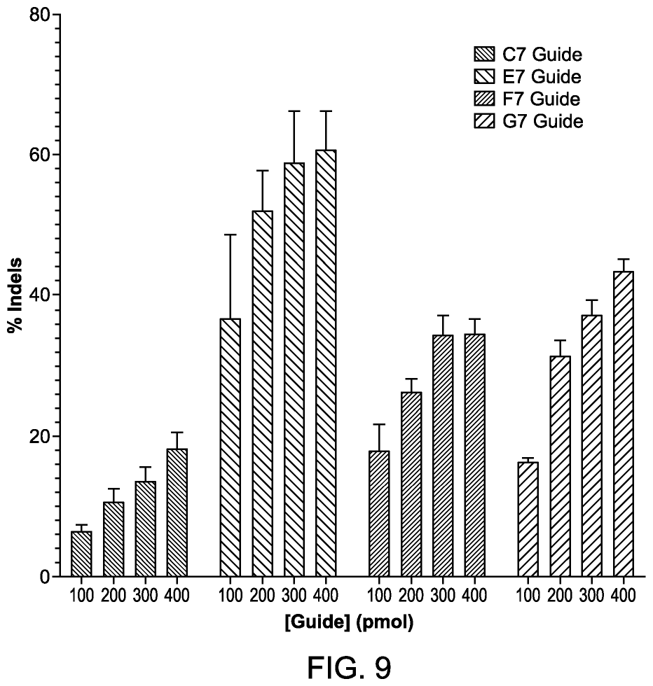

Guides C7, E7, F7, and G7 (SEQ ID NOs: 1402, 1407, 1410, and 1412) were selected for dose titration experiments in cells. In this experiment, the sgRNA dose was varied from 100 pmol to 400 pmol to test for editing saturation. As shown in FIG. 9, the editing efficiency increases with increasing guide, plateauing at 400 pmol for some guides. Results highlight the potential of protein sequence reconstruction for diversifying the biochemical properties and activity of SMART I nucleases.

### Example 4—Diversification of SMART I Effector Sequences Generates Active Nucleases

Given the results observed for the MG34 family of nucleases, the use of ancestral sequence reconstruction (ASR) methods was extended to the SMART I MG102 family of nucleases for protein sequence diversification. In addition, the development of novel algorithms based off of ASR was explored to generate additional diversity of the SMART MG34 family. The activity of these computational protein reconstructions was tested in in vitro PAM enrichment experiments.

### Computational Reconstruction of SMART Ancestral Intermediate Sequences

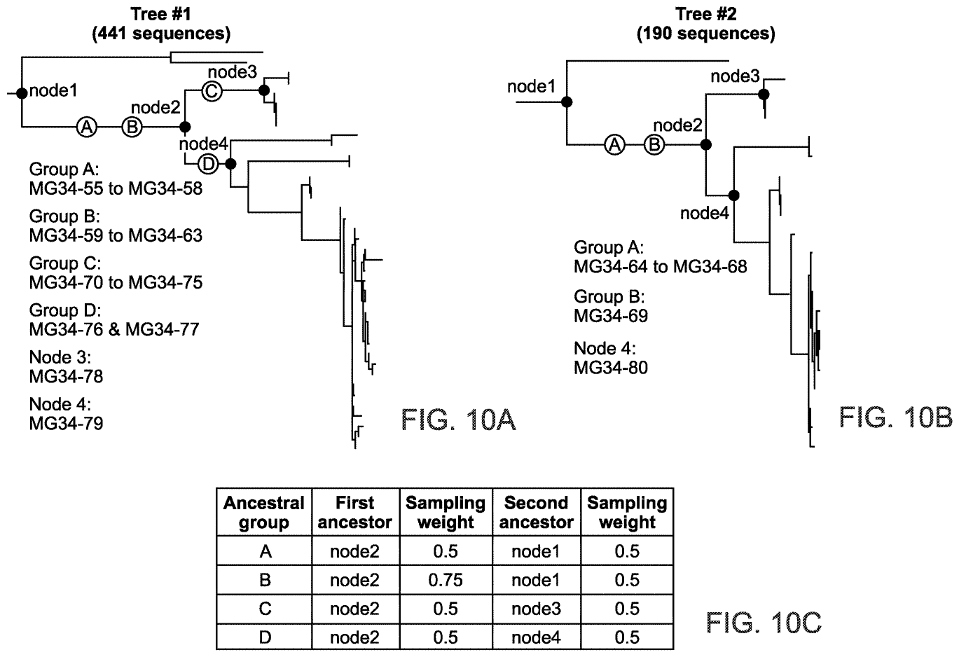

To generate additional diversity of SMART nucleases, ancestral sequence reconstruction was used to generate sequences that represent potential ancestral intermediates. For this analysis, two phylogenetic trees with reconstructed ancestors were used to generate sequences that represent hybrids between two different reconstructed ancestral tree nodes. The first tree contained 441 SMART I protein sequences aligned and a phylogenetic tree built (FIG. 10A). The second tree contained 190 SMART I protein sequences aligned, and a tree was built (FIG. 10B). Both trees were rooted using SpCas9 and SaCas9, sequence reconstruction was done, and insertions and deletions were identified manually for each reconstructed node.

To generate putative ancestral intermediates, amino acids were randomly selected from one ancestor to be introduced into another ancestor, using pre-defined weights for the random selection (FIG. 10C). General weights were assigned to each ancestor (FIG. 10C), and subsequently the probabilities of all twenty amino acids from the original ancestral reconstruction algorithm were used to determine which amino acid from the second ancestor to introduce into the first ancestor. Four different groups of ancestral intermediates were generated: those that represent on average 50% of node 1 and 50% of node 2 (Group A, SEQ ID NOs: 1415-1418 and 1424-1428), 75% of node 2 and 25% of node 1 (Group B, SEQ ID NOs: 1419-1423 and 1429), 50% of node 2 and 50% of node 3 (Group C, SEQ ID NOs: 1430-1435), and 50% of node 3 and 50% of node 4 (Group D, SEQ ID NOs: 1436-1437) (FIG. 10C). In addition, ancestors corresponding to nodes 3 and 4 were also generated (SEQ ID NOs: 1438-1440).

### In Vitro PAM Determination Assays

Candidate MG34 and MG102 effectors were tested in vitro for nuclease activity and PAM preference, as described previously (Example 1). For ancestral MG34 nucleases, the sgRNA sequences used belong to two active MG34 homologs: MG34-1 (sg1) (SEQ ID NO: 613) and MG34-25 (sg25) (SEQ ID NO: 1369). For ancestral MG102 sequences, the sgRNA from three active homologs were used: MG102-2 (sg1) (SEQ ID NO: 1013), MG102-39 (sg39) (SEQ ID NO: 1017), and MG102-42 (sg42) (SEQ ID NO: 1018). For MG102 native sequences recovered here, four single guide designs were tested per protein (SEQ ID NOs: 1376-1391), and for MG34 native sequences, eight single guide designs were tested (SEQ ID NOs: 1392-1399). The different sgRNA designs included different spacer lengths, spacers, and trim positions in the CRISPR repeat and predicted tracrRNA.

### Experimental Results

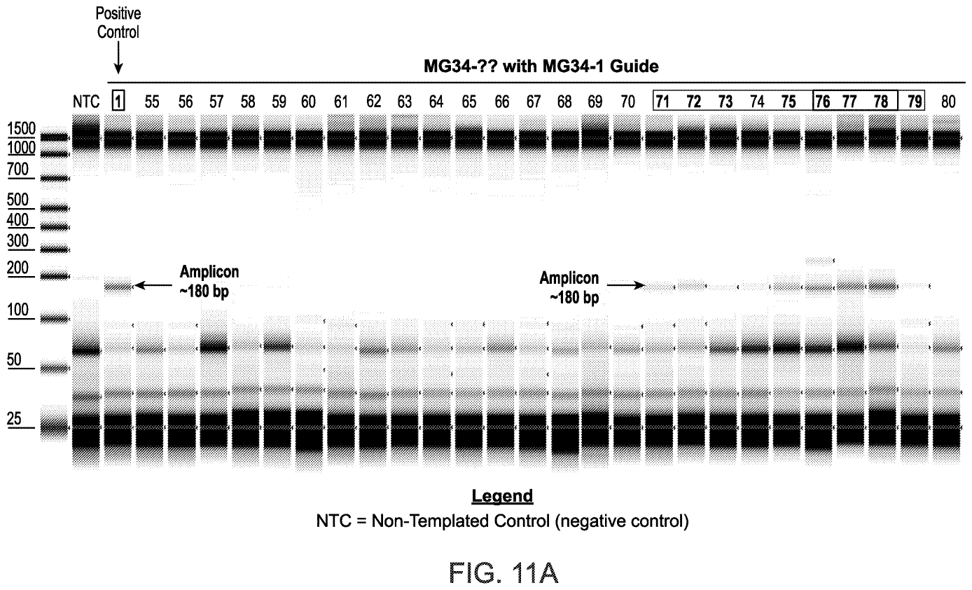

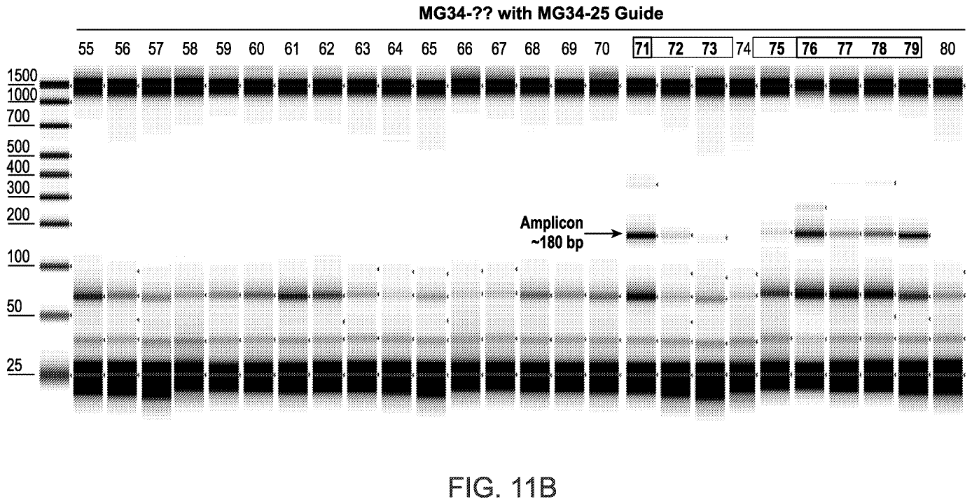

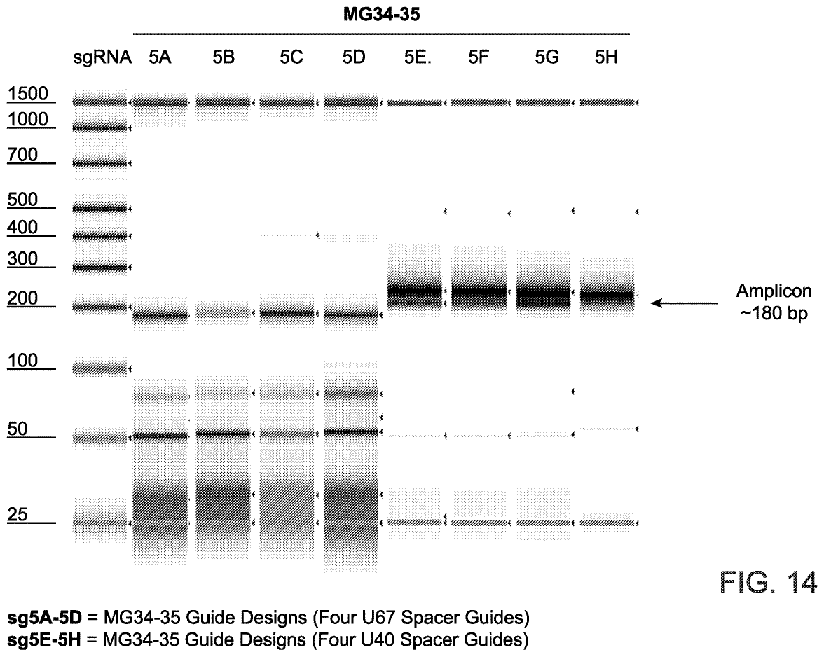

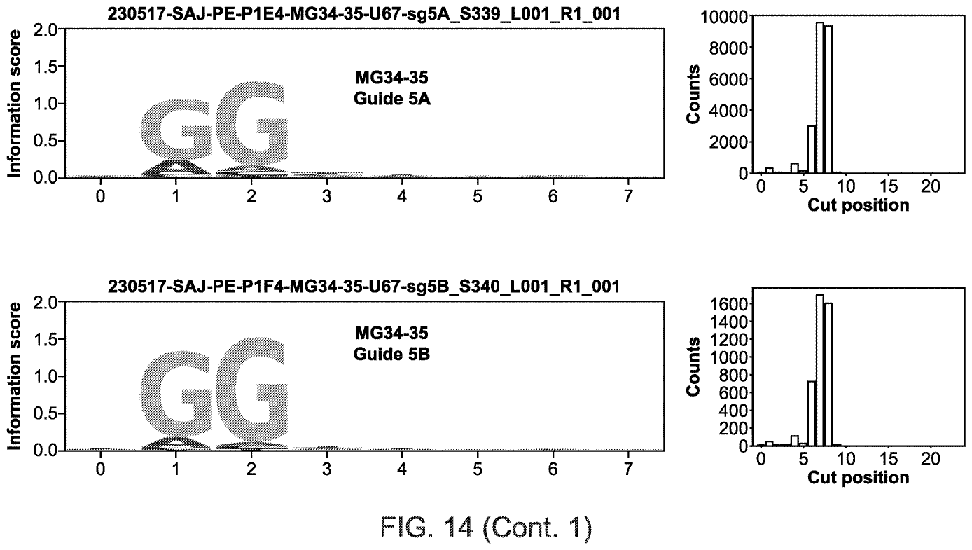

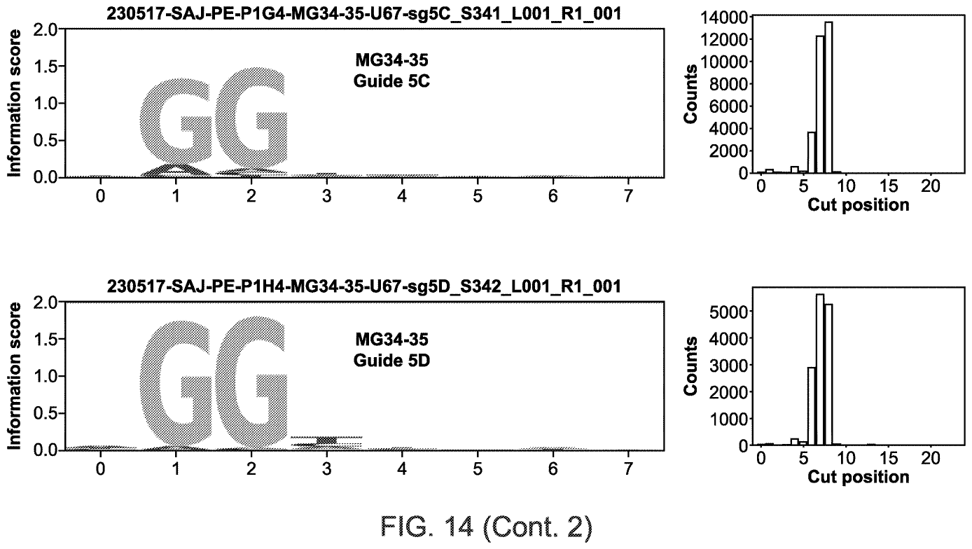

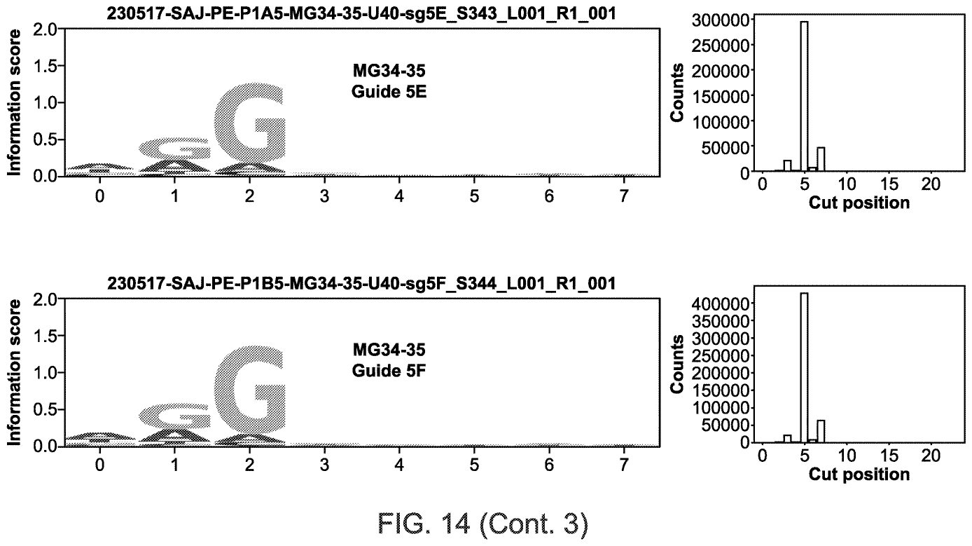

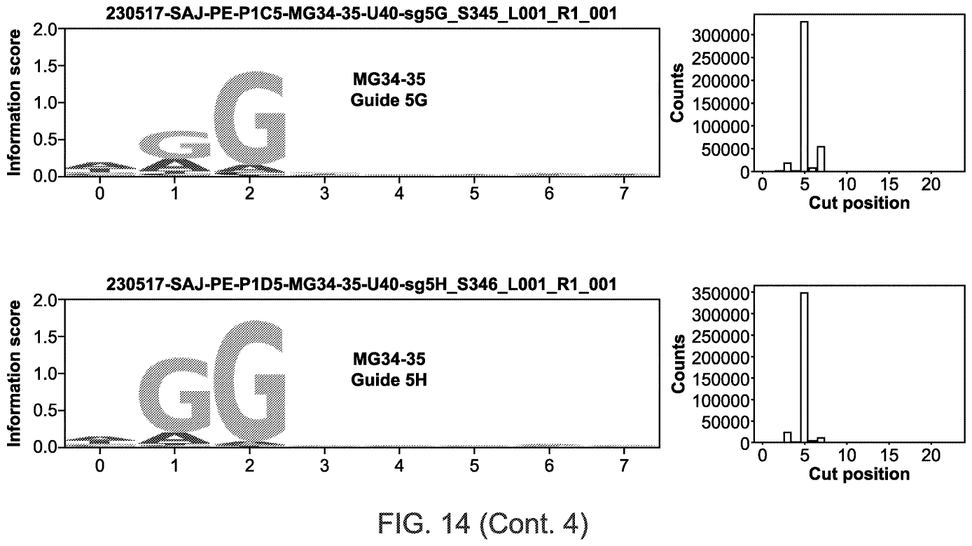

Nine new MG34 ancestral nucleases were found to be active in vitro using the guide RNAs from active MG34 nucleases: MG34-71 through MG34-79 (SEQ ID NOs: 1431-1439) (FIGS. 11A-11B, 12, and 13). The PAMs and cut sites are listed in Table 8. In addition, one native MG34 candidate, MG34-35 (SEQ ID NO: 1347) was found to be active in vitro using different sgRNA designs (FIG. 14).

TABLE 8
In vitro PAM and cleavage site for active MG34
family nucleases

| Protein | PAM | SEQ ID NO for PAM |
|---|---|---|
| MG34-35 | NRG | 1441 |
| MG34-71 | NGG | 1442 |
| MG34-72 | NGG | 1443 |
| MG34-73 | NGGT | 1444 |
| MG34-74 | NGGT | 1445 |
| MG34-75 | NGGT | 1446 |
| MG34-76 | NGGT | 1447 |
| MG34-77 | NGGTNNA | 1448 |
| MG34-78 | NGG | 1449 |
| MG34-79 | NGG | 1450 |

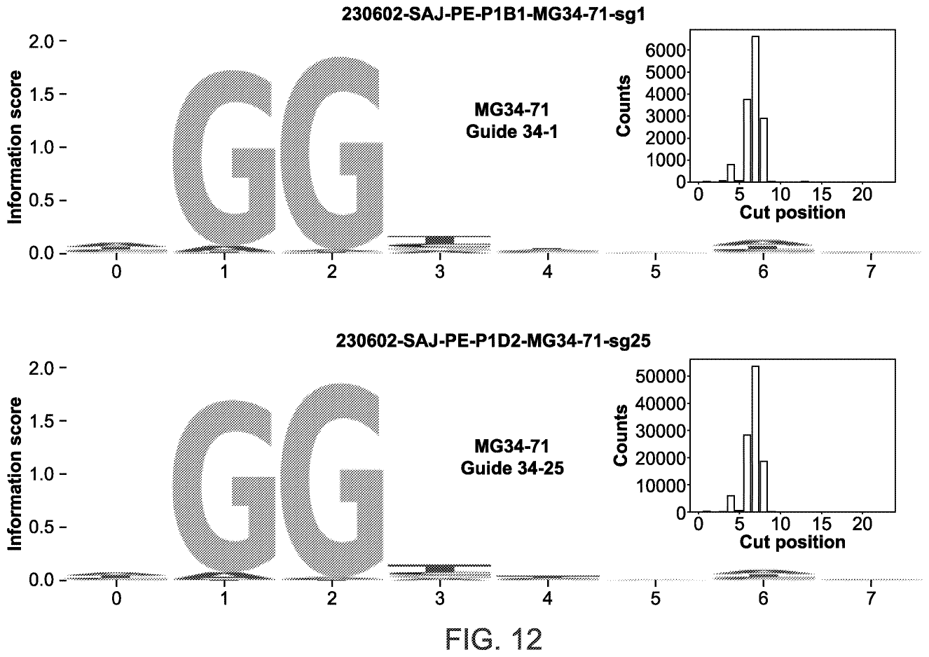

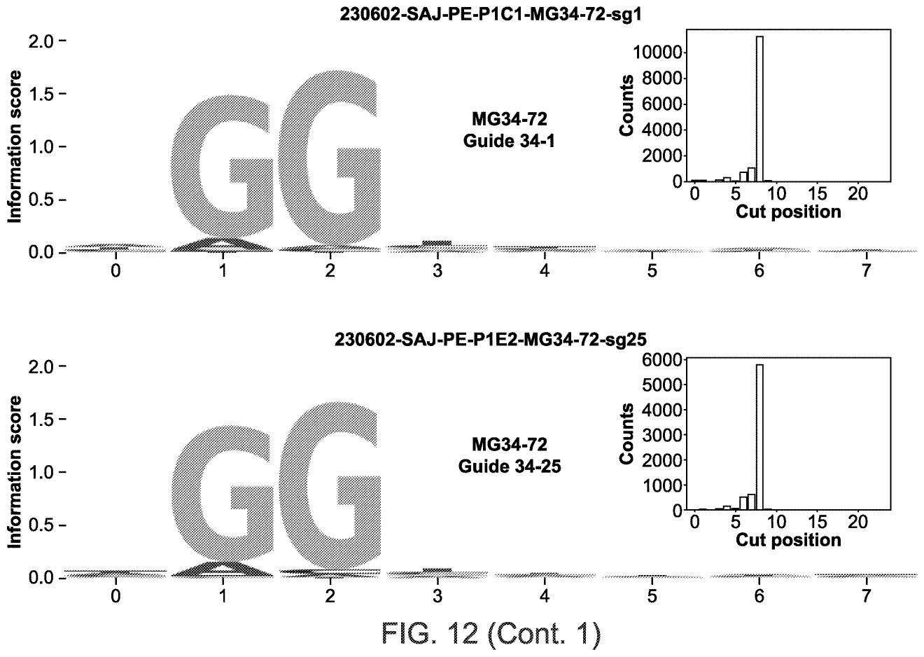

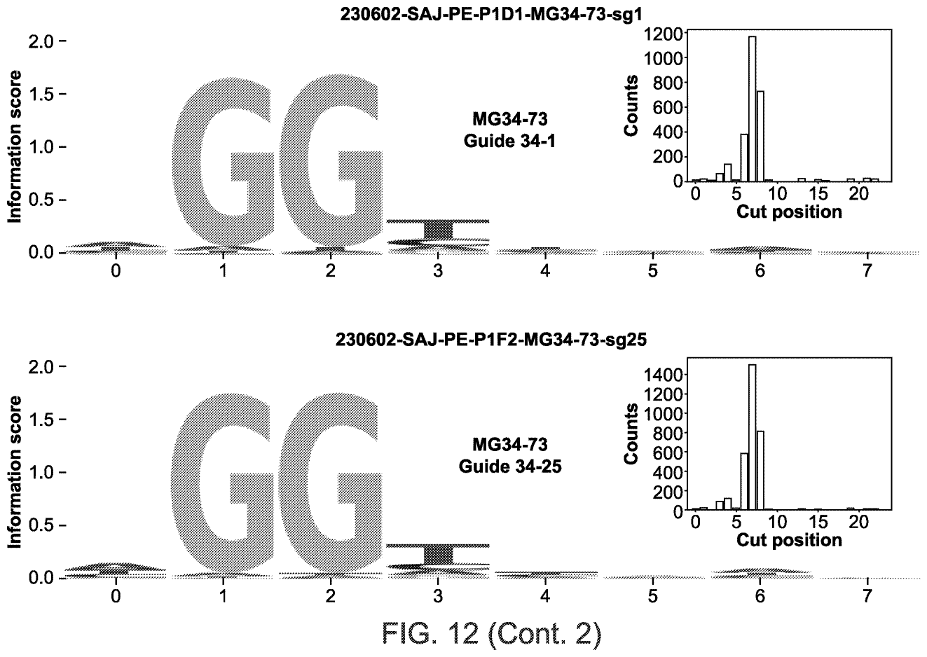

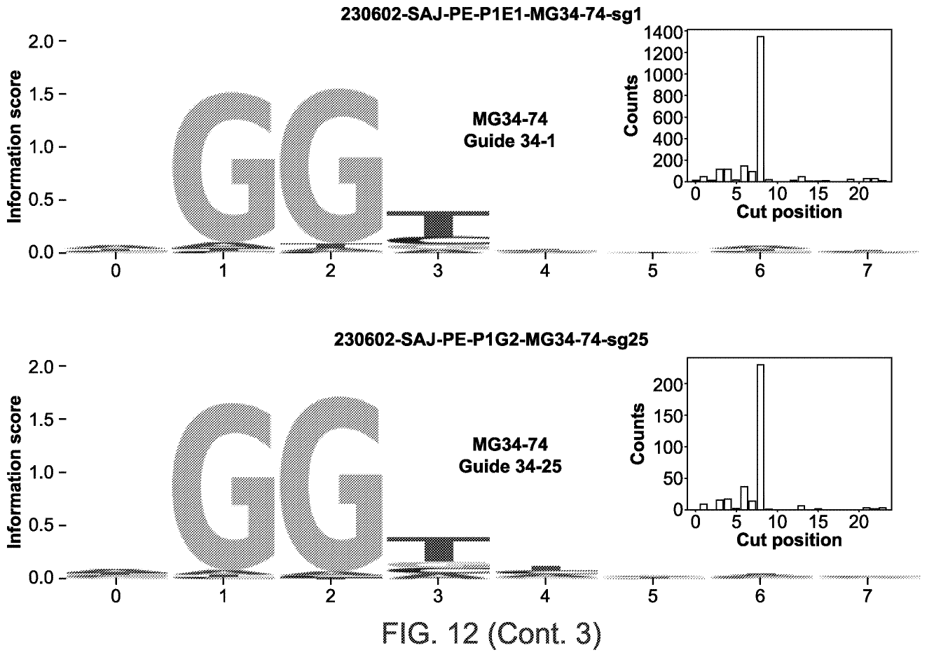

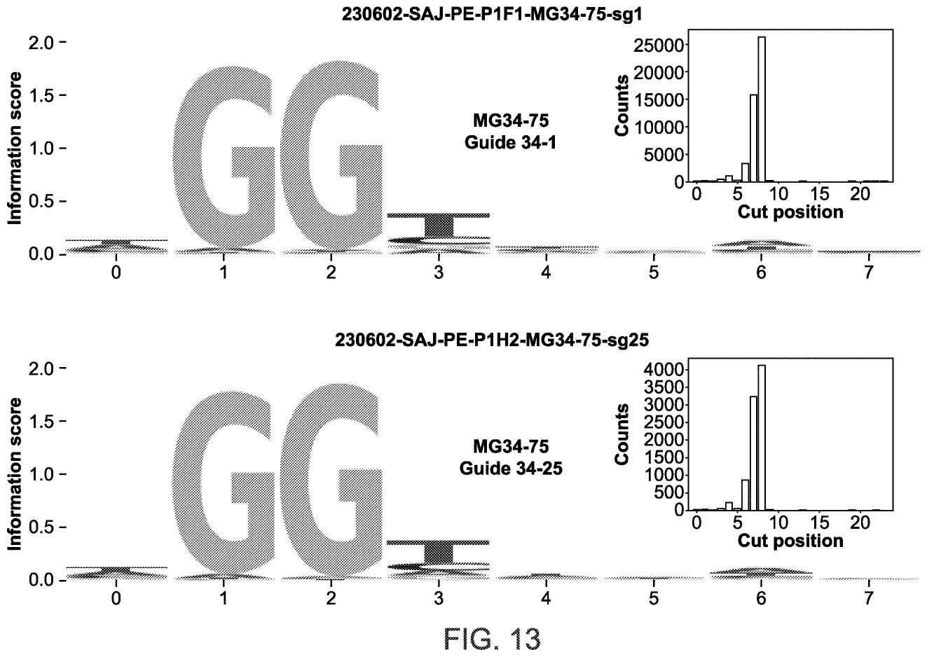

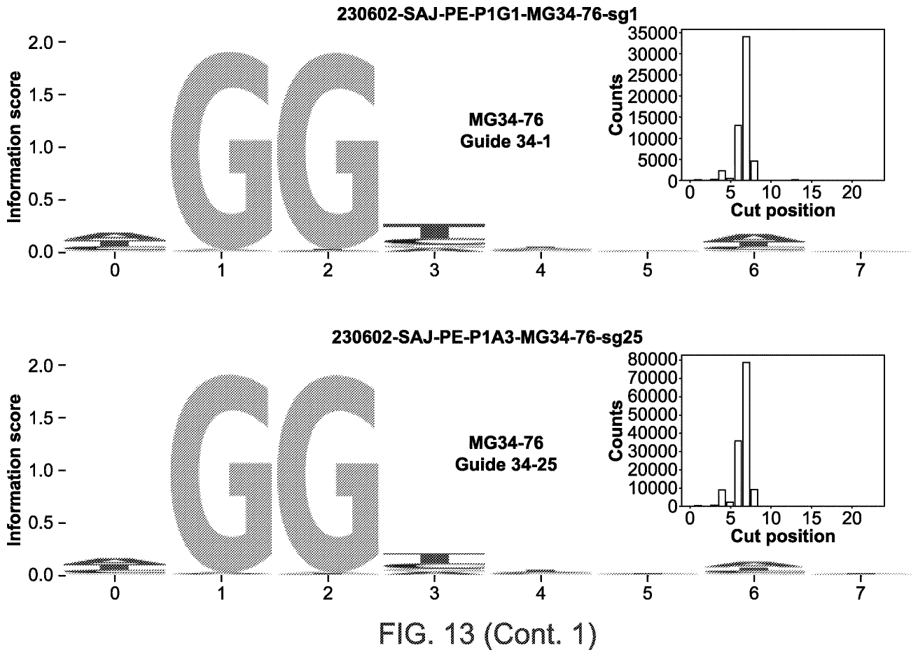

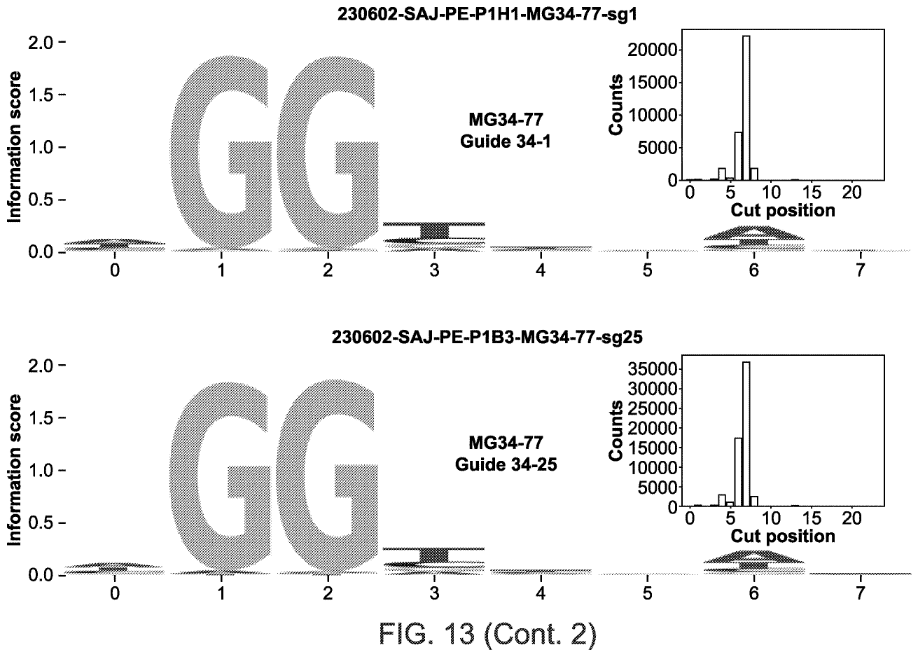

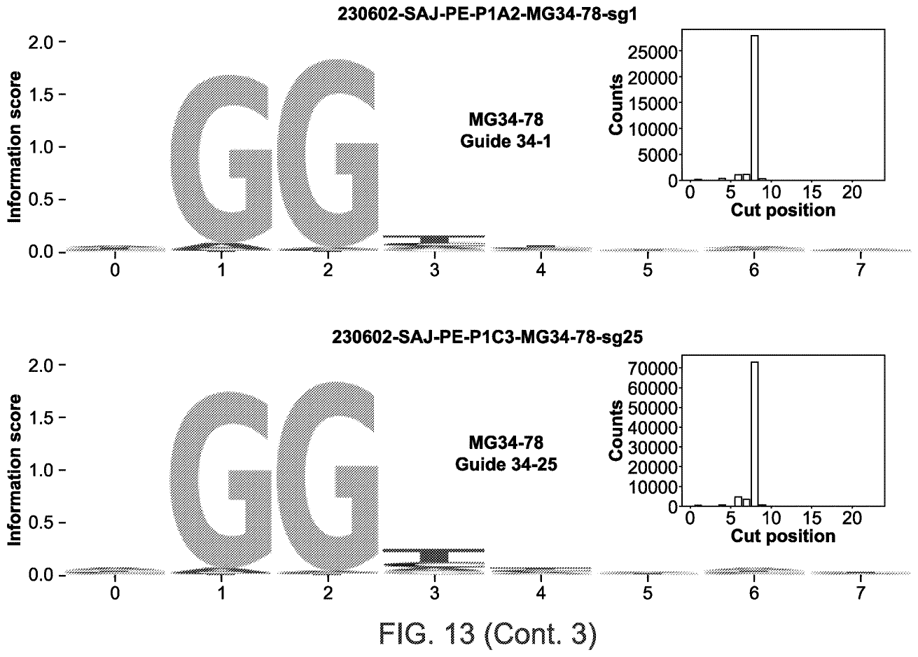

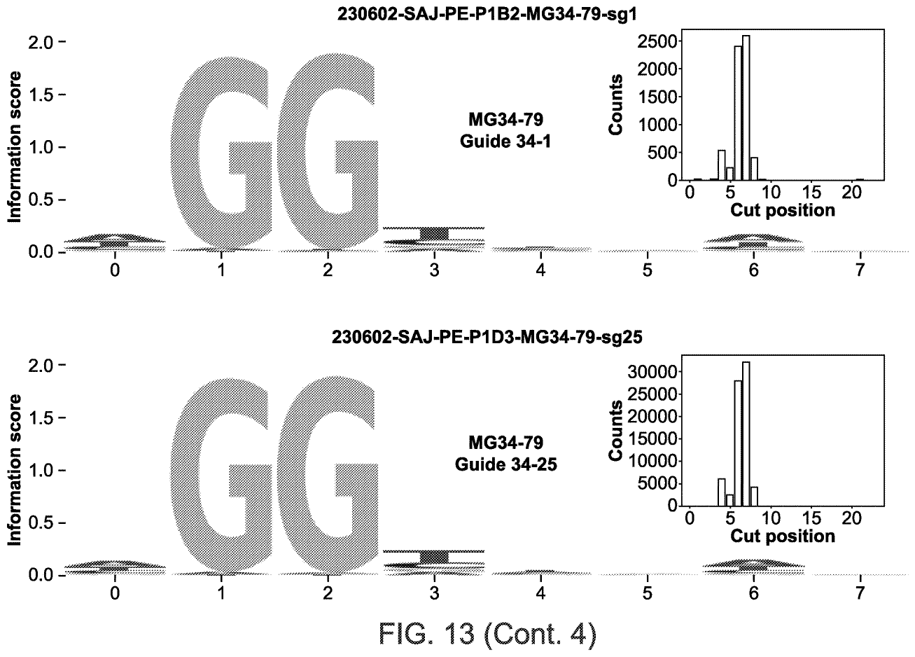

The 3′ PAM motif nGG appears to be the most commonly recognized by ancestral nucleases of the MG34 family (FIGS. 12-13), while nGG or nRG is recognized by MG34-35 (FIG. 14). Some nucleases show weak preferences in the 4th base position, for example NGGT over NGGN where a T is more enriched in cleavage products but not strictly required. All exhibit a preference for cleavage at positions 5-8 from the PAM (FIGS. 12-14).

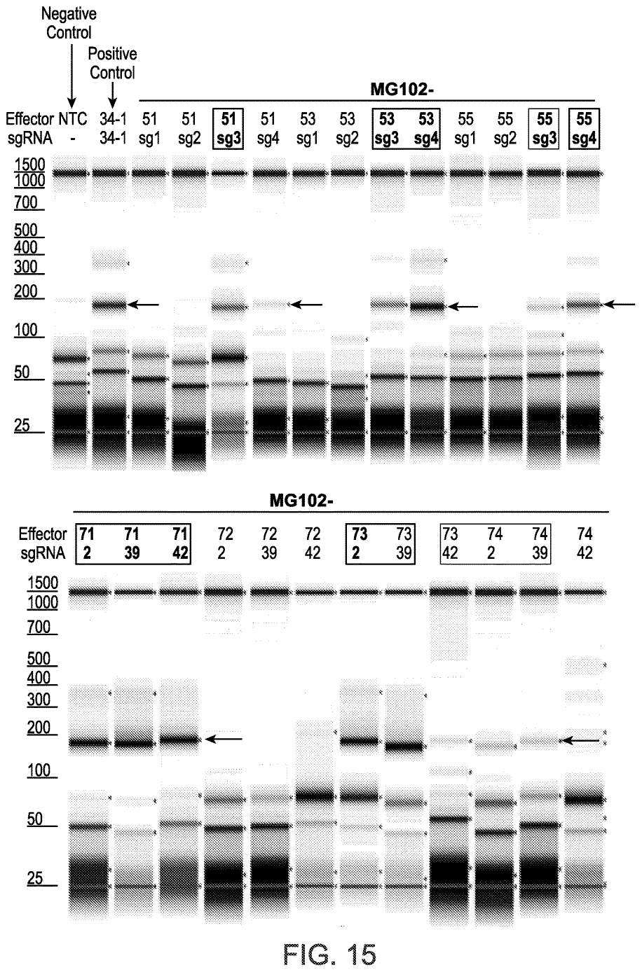

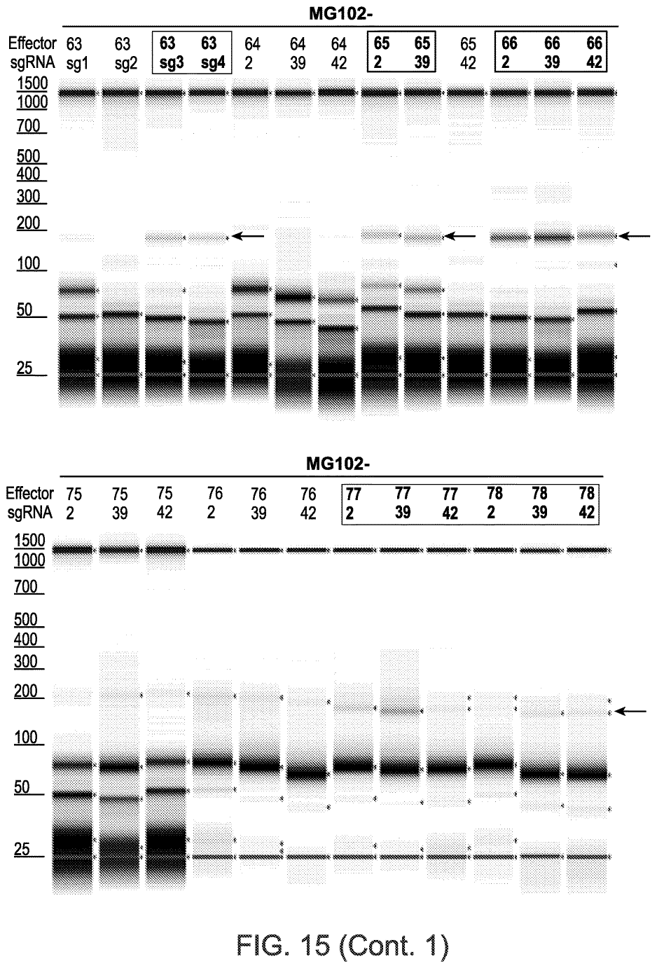

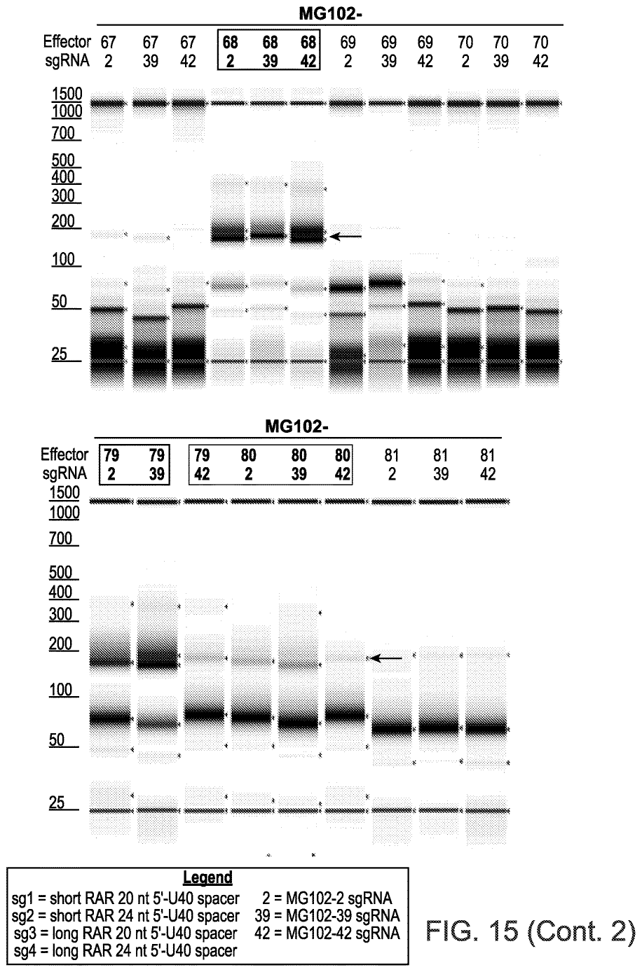

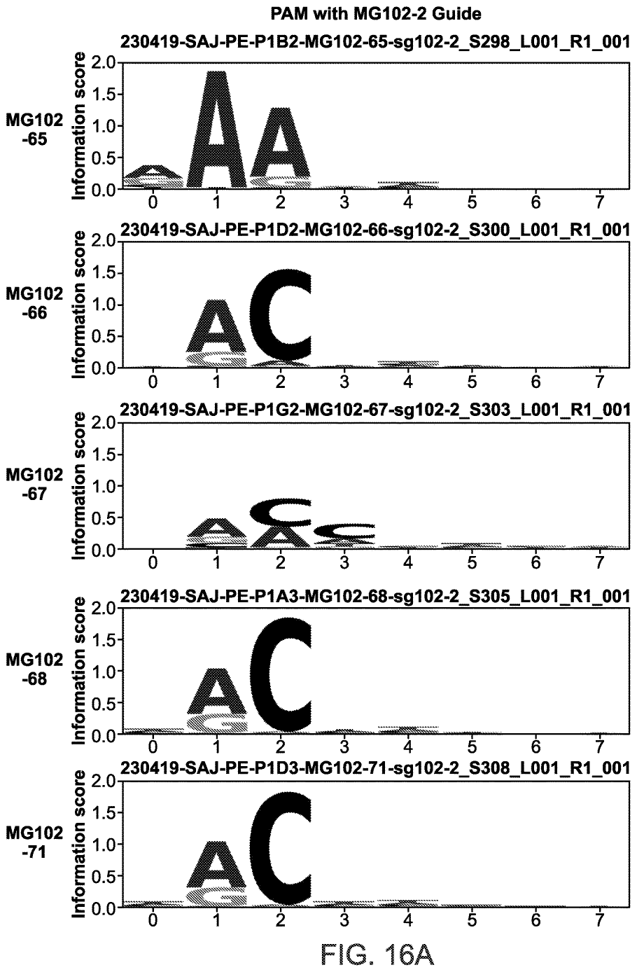

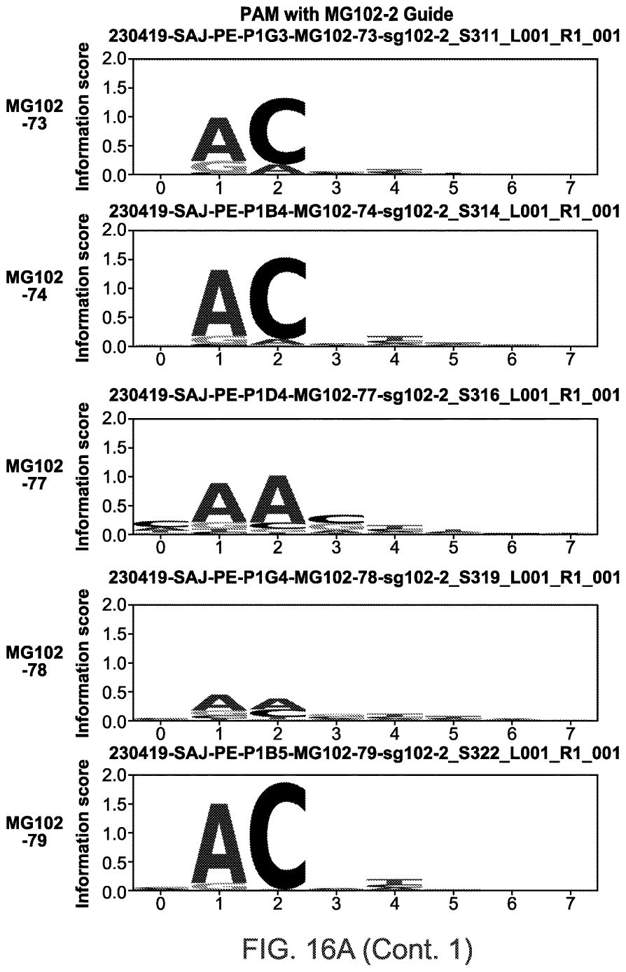

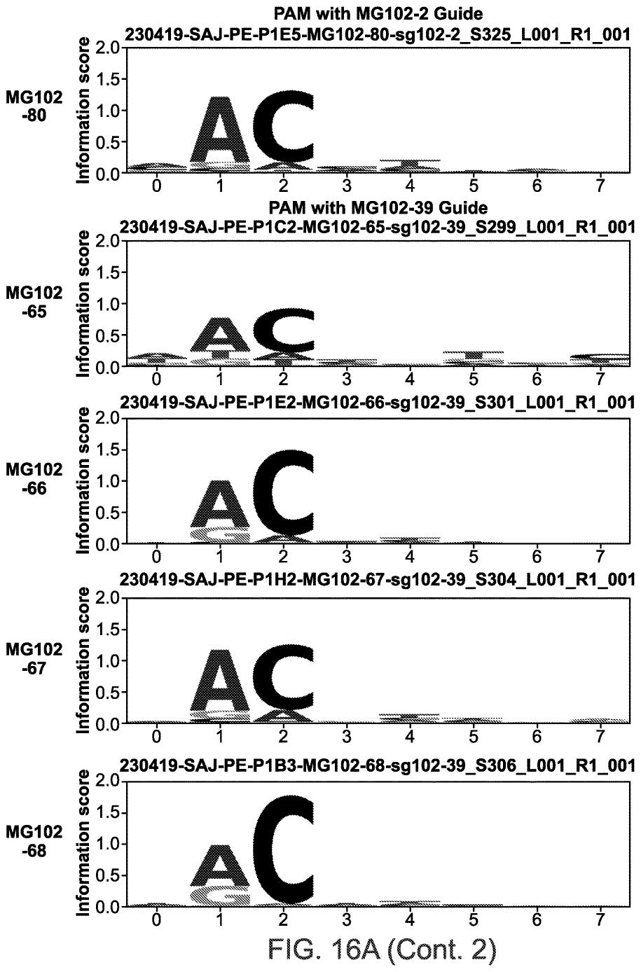

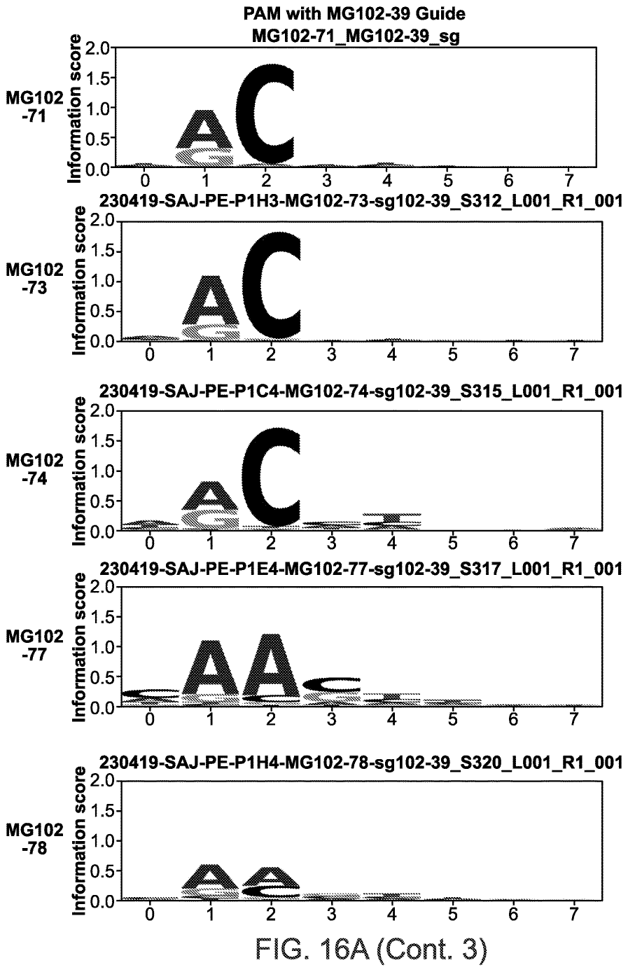

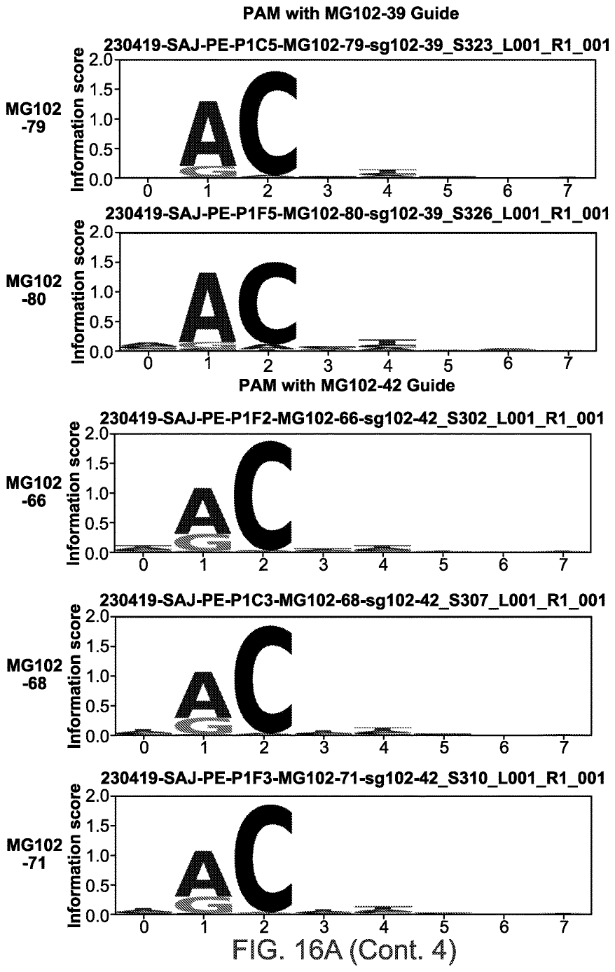

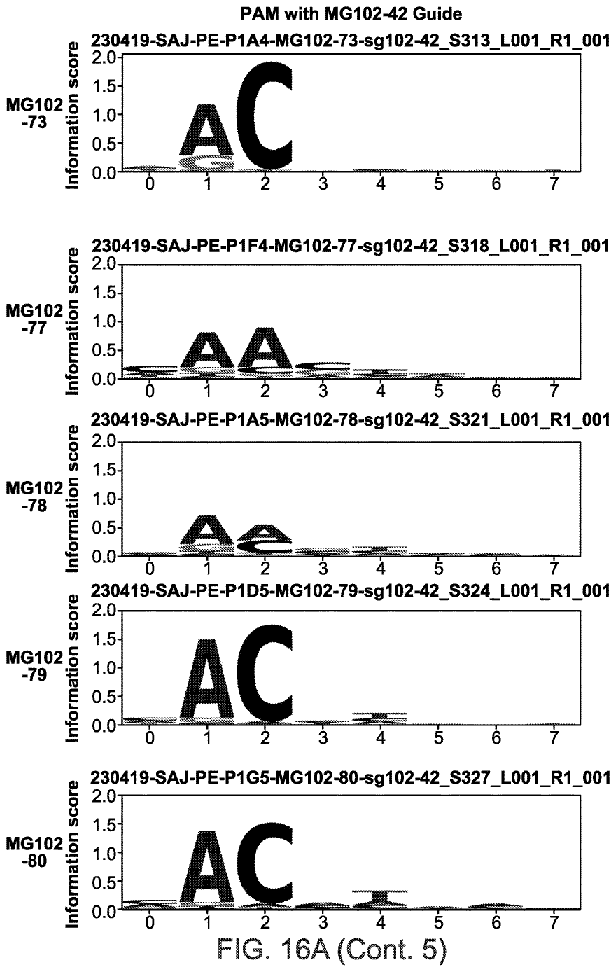

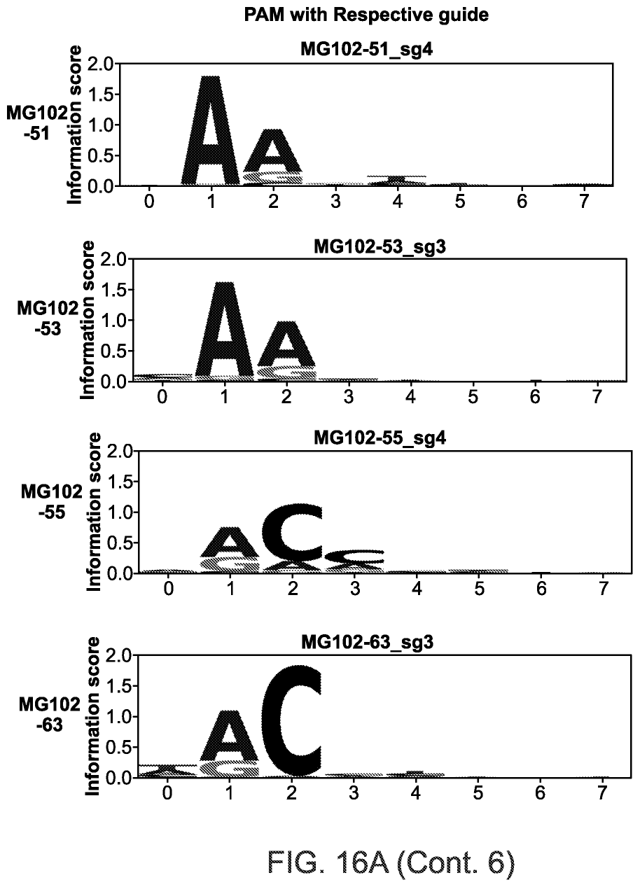

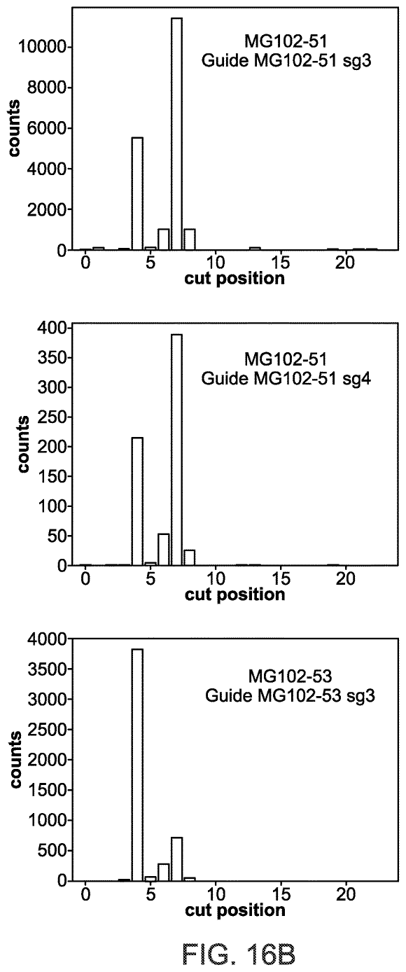

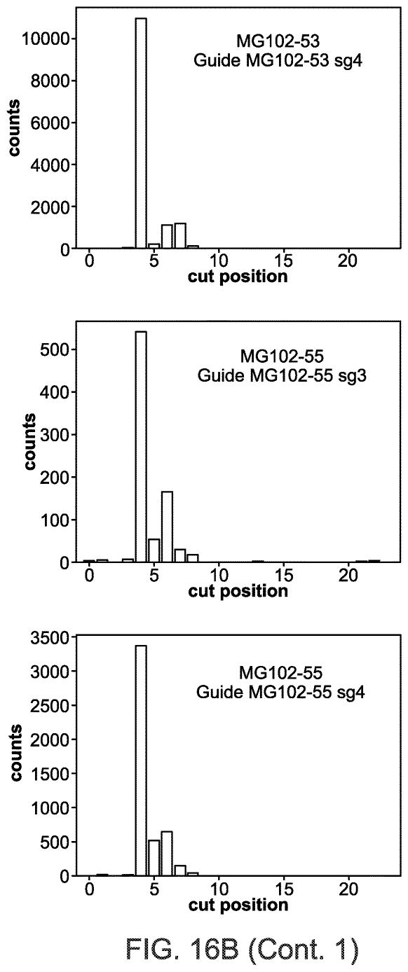

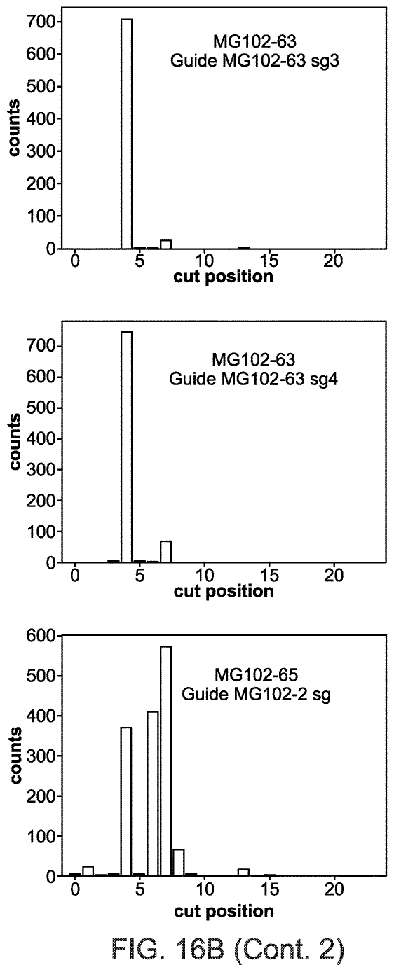

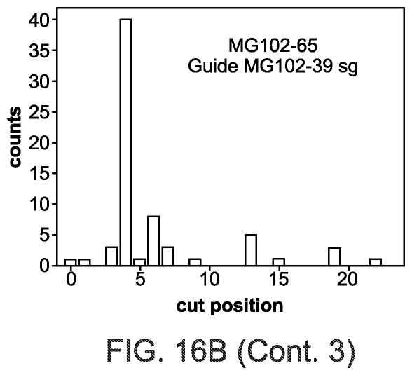

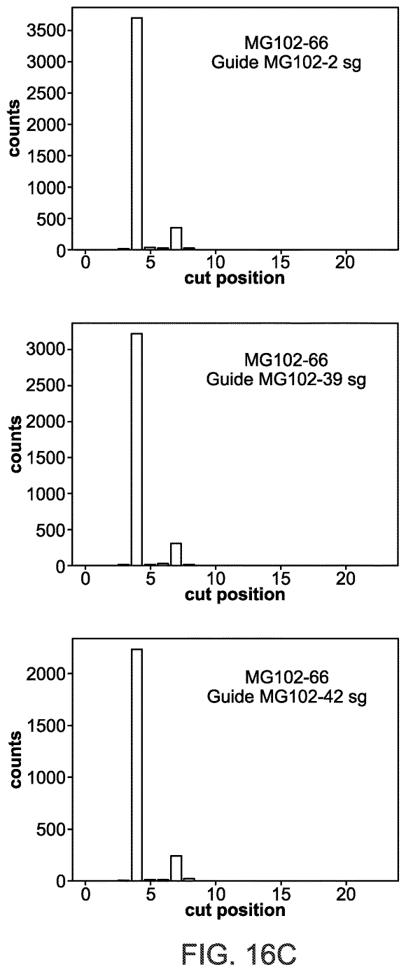

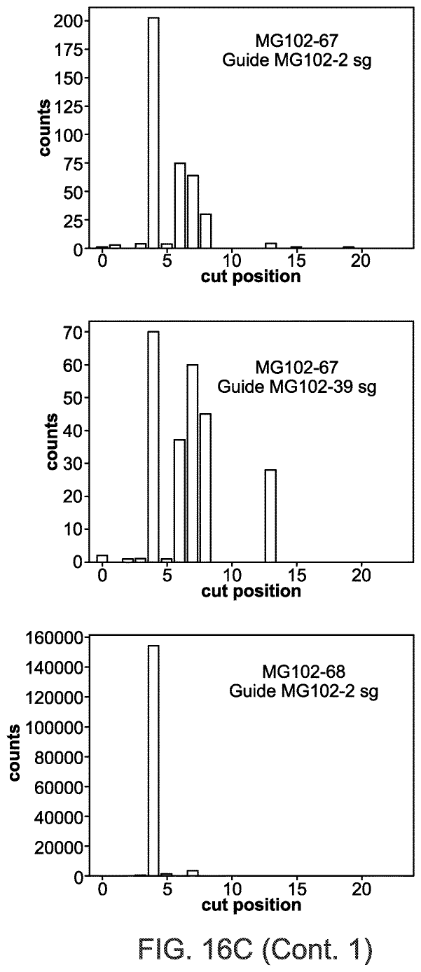

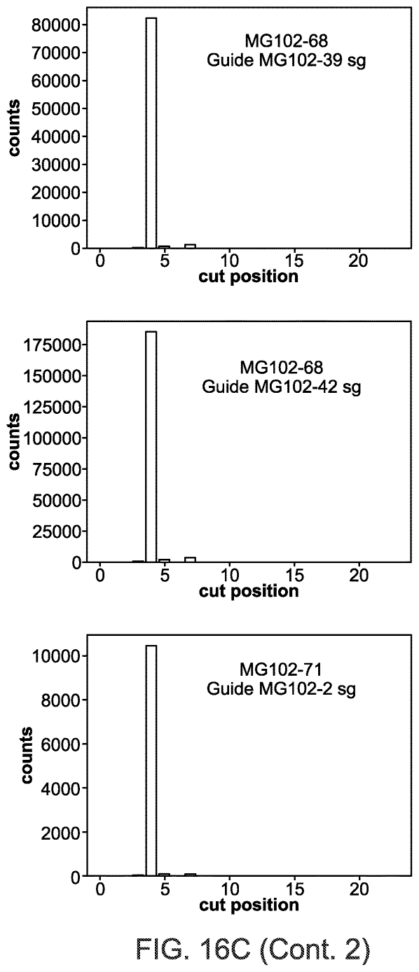

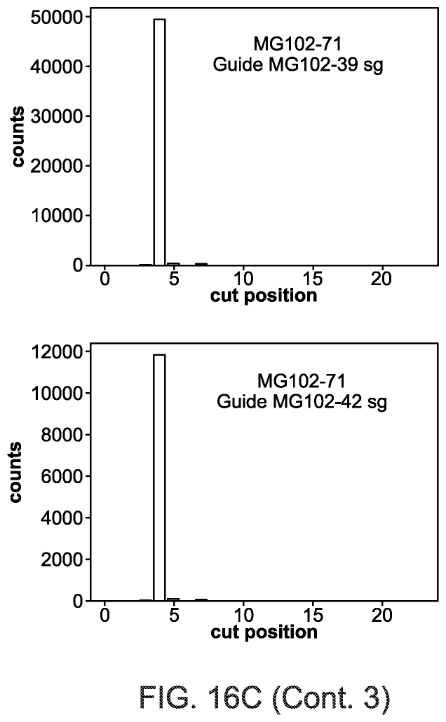

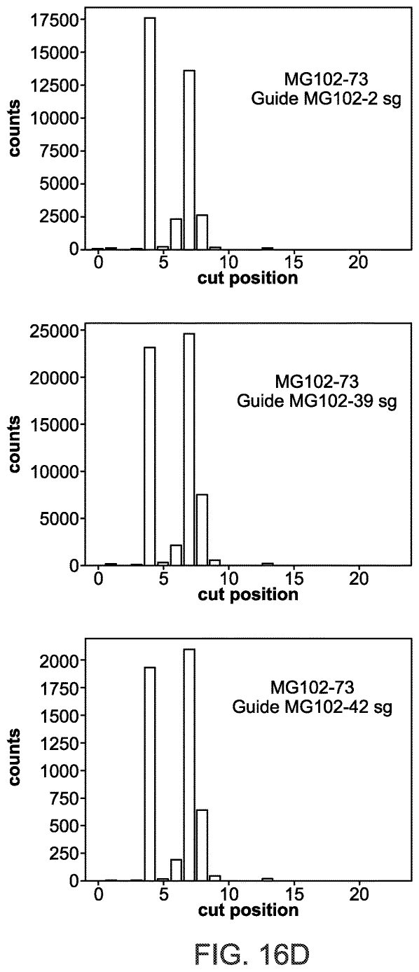

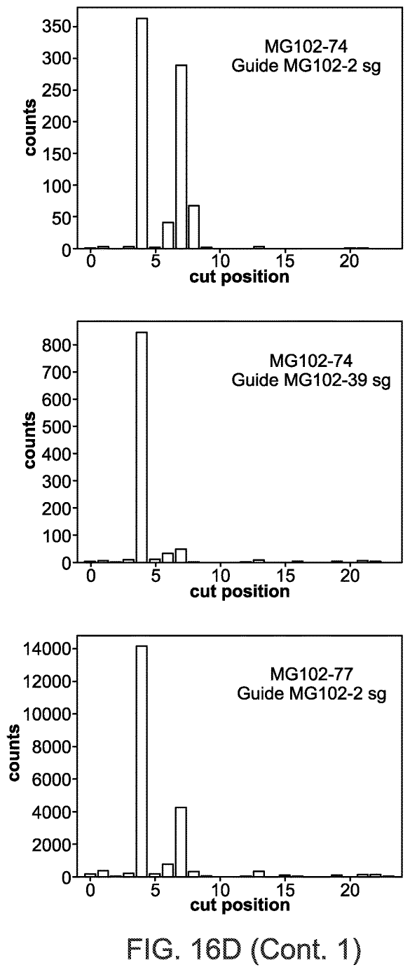

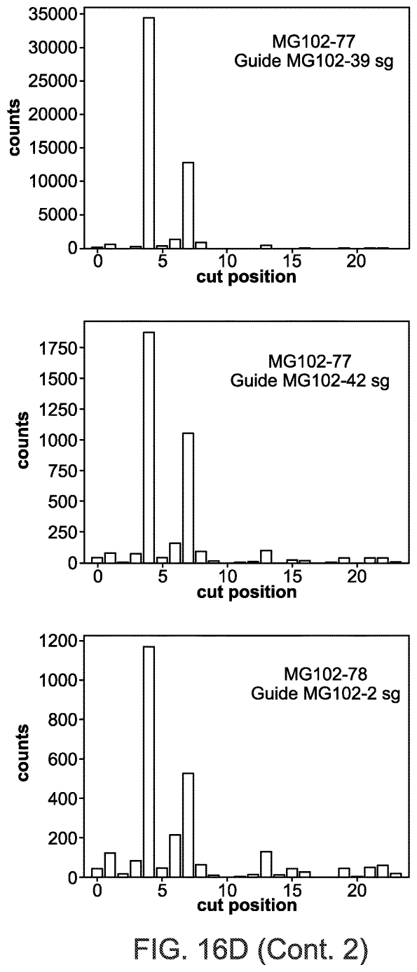

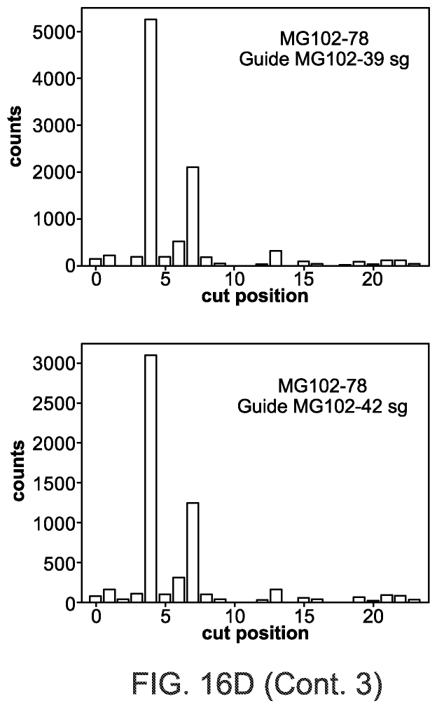

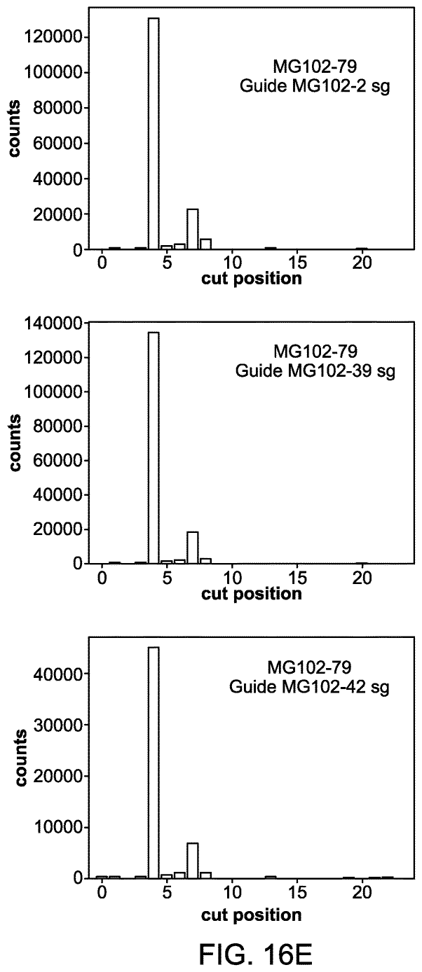

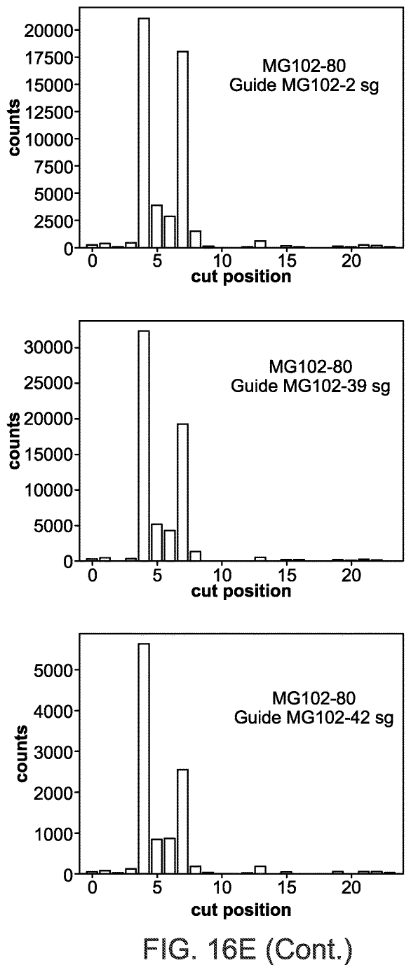

In addition, fifteen new MG102 nucleases were found to be active in vitro and recognize a variety of 3′ PAM sequences: the native nucleases MG12-51 (SEQ ID NO: 1262), MG102-53 (SEQ ID NO: 1264), MG102-55 (SEQ ID NO: 1266), and MG102-63 (SEQ ID NO: 1324), and the ancestral nucleases MG102-65 through MG102-68 (SEQ ID NOs: 1330-1333), MG102-71 (SEQ ID NO: 1336), MG102-73 (SEQ ID NO: 1338), MG102-74 (SEQ ID NO: 1339), and MG102-77 through MG102-80 (SEQ ID NOs: 1342-1345) (FIGS. 15 and 16A, Table 9).

TABLE 9
In vitro PAM and cleavage site for active MG102
nucleases

| | | SEQ ID |
|---|---|---|
| Protein | PAM | NO for PAM |
| MG102-51 | NAR | 1451 |
| MG102-53 | NAR | 1452 |
| MG102-55 | NRMC | 1453 |
| MG102-63 | WRC | 1454 |
| MG102-65 with MG102-2_sg | RAR | 1455 |
| MG102-65 with MG102-39_sg | NRM | 1456 |
| MG102-66 | NRC | 1457 |
| MG102-67 with MG102-2_sg | NAMC | 1458 |
| MG102-67 with MG102-39_sg | NRM | 1459 |
| MG102-68 | NRC | 1460 |
| MG102-71 | NRC | 1461 |
| MG102-73 | NRM | 1462 |
| MG102-74 | NRCNT | 1463 |
| MG102-77 | CRMS | 1464 |
| MG102-78 | NRM | 1465 |
| MG102-79 | NRC | 1466 |
| MG102-80 with MG102-2_sg | NAM | 1467 |
| MG102-80 with MG102-39_sg | NRC | 1468 |
| MG102-80 with MG102-42_sg | NACNT | 1469 |

These nucleases all show cleavage preferences between 4-8 bases from the PAM, with 4 or 7 being the most frequent sites of cleavage (FIGS. 16B-16E). These results demonstrate that computational diversification of protein sequences successfully generates active nucleases with different targeting abilities.

### Example 5—SMART I Nucleases are Active Gene Editors in Human Cells

Here, the testing of the active nuclease MG34-29 at different genetic loci and with different guide scaffolds was expanded. In addition, the activity of new ancestral nucleases (MG34-71, MG34-72, MG34-79, MG102-68, MG102-71) as well as two new native nucleases (MG34-35 and MG102-53) were evaluated in human cells.

mRNA Production

Sequences for nucleases were codon optimized for human expression and cloned into an expression vector with a CleanCap T7 promoter, 5′ and 3′ UTRs, and a polyA tail. Additionally, some plasmids containing nucleases codon optimized for human expression were ordered in an expression vector and a CleanCap T7 promoter and 5′-UTR, and separately a 3′-UTR and polyA tail, were added via PCR primers. The coding sequences contained an N-terminal SV40 nuclear localization signal and a C-terminal nucleoplasmin nuclear localization signal. Cloned expression vectors for nucleases were midi-prepped), linearized with SapI, and used for in vitro transcription with Hi-T7. PCR amplified nuclease coding sequences were directly used for in vitro transcription following the same Hi-T7 methods. In vitro transcription reactions contained N1-methylpseudouridine in place of uridine and had added CleanCap reagent. The resulting mRNA was checked for product size and purity via Tapestation and diluted to 250 ng/μL in sterile water for use in nucleofection.

### Nucleofection

K562 cells were cultured in IMDM media+10% FBS for 1-2 passages prior to nucleofection. On the day of nucleofection, cells were harvested, counted, washed in 1×PBS, and resuspended in SF buffer. 120,000 cells were distributed per well and nucleofected with 500 ng of mRNA and 200 (MG34s) or 450 (MG102s) pmol of sgRNA using a SF Cell Line 96-well Nucleofector™ Kit. Cells were added to recovery media and grown for 72 hours before genomic DNA was harvested with QuickExtract. Resulting gDNA was diluted 1:3 and used as a template for NGS PCR. Indels were quantified from resulting NGS reads mapped to the reference amplicon.

### Experimental Results

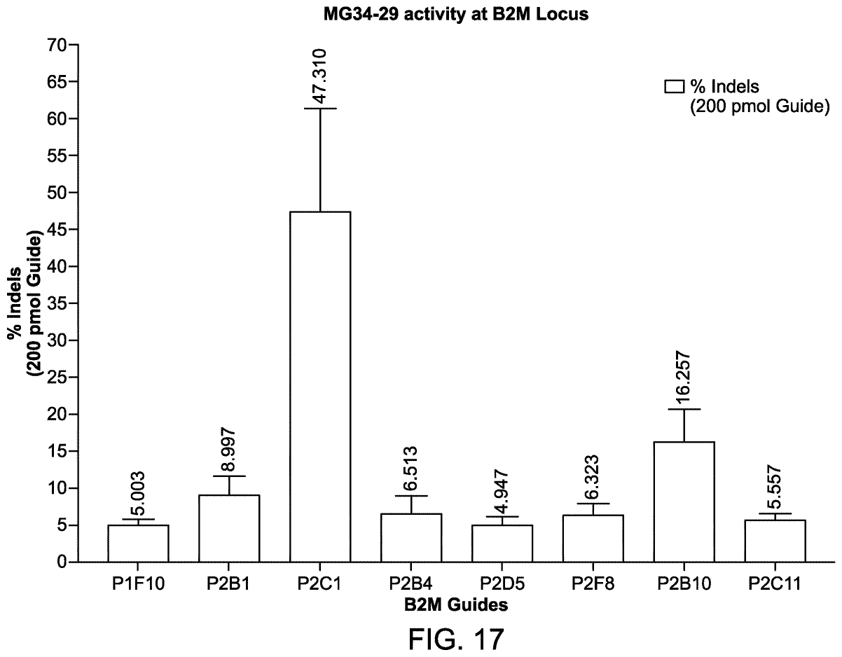

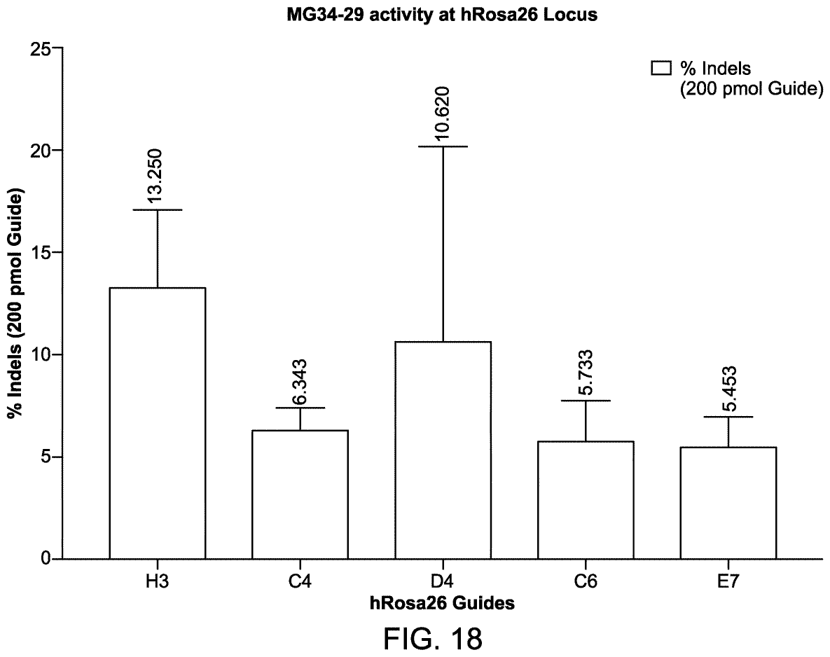

The ancestral nuclease MG34-29 (SEQ ID No. 1316) was found to be active with guides with the MG34-1 guide backbone and NGG PAMs targeting three new genetic loci: Beta-2 microglobulin (B2M) (FIG. 17), hRosa2 (FIG. 18), and TRAC. For B2M, 192 guides were tested and eight (8) showed an activity of >5% indel formation, with maximum editing of 47% (FIG. 17, Table 10). For hRosa2, 72 guides were tested and five (5) showed an activity of >5% indel formation, with maximum editing of 13% (FIG. 18, Table 10). For TRAC, 49 guides were tested and one (1) showed an activity of >5% indel formation, with maximum editing of 8% (Table 10).

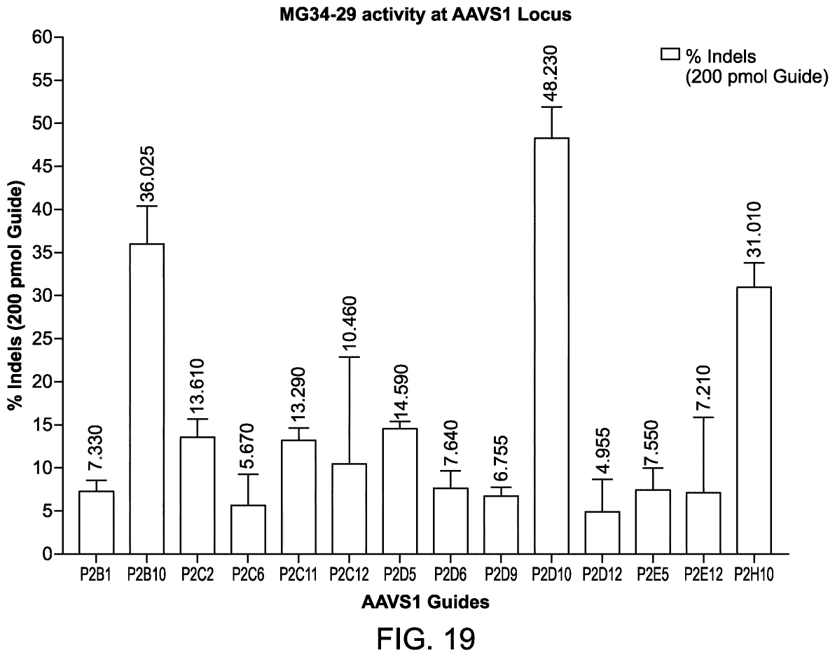

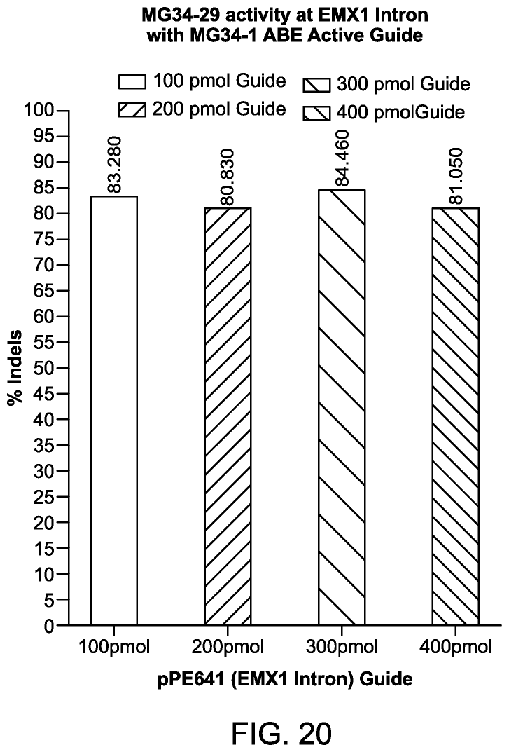

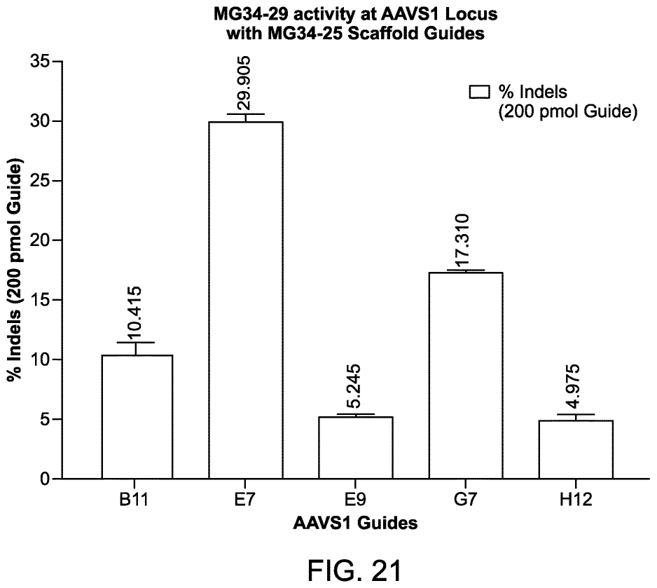

MG34-29 was also found to be active with additional MG34-1 guide backbone NGG PAM guides at AAVS1 (FIG. 19). An additional 100 guides were tested and 14 showed activity >5% indel formation, with maximum editing of 48% (FIG. 19, Table 10). MG34-29 was also tested with 4 guides targeting diverse loci designed to test MG34-1 and guide pPE641 targeting the EMX1 intron demonstrated high in-cell editing >80% indels, but did not demonstrate a dose-dependent response when increasing sgRNA concentrations up to 400 pmol (FIG. 20, Table 10). MG34-29 was also found to be active with MG34-25 guide backbone NGG PAM guides at AAVS1, with maximum editing of 29% (FIG. 21, Table 10).

TABLE 10
MG34-29 guides with >5% indel formation at B2M, hRosa26, TRAC, or AAVS1

| | | | sgRNA | Target site |
|---|---|---|---|---|
| | sgRNA | | SEQ ID | in genome |
| Locus | well | Target site in genome | NO: | SEQ ID NO |
| B2M | P1F10 | GGCCTACGGCGACGGGAGGGTC | 1571 | 2270 |
| B2M | P2B1 | CAGCGCAGCTGGAGTGGGGGAC | 1591 | 2271 |
| B2M | P2C1 | GCAGCTGGAGTGGGGGACGGGT | 1592 | 2272 |
| B2M | P2B4 | GTTCGCATGTCCTAGCACCTCT | 1615 | 2273 |
| B2M | P2D5 | GTGTGGCCCCACATAGACCCAG | 1625 | 2274 |
| B2M | P2F8 | CCGCTTCCCCGAGATCCAGCCC | 1651 | 2275 |
| B2M | P2B10 | AGAGCACAGCGAGGGCCACAGA | 1663 | 2276 |
| B2M | P2C11 | GGAAGGACCAGAGCGGGAGGGT | 1672 | 2277 |
| hRosa26 | H3 | GATGCAAAGGGGTAGAGAGCCT | 1709 | 2278 |
| hRosa26 | C4 | GGGTAGAGAGCCTGGGGAGAGA | 1712 | 2279 |
| hRosa26 | D4 | GAGAGCCTGGGGAGAGAGGGTG | 1713 | 2280 |
| hRosa26 | C6 | TTACAGCTGCTCACTCCCCTGC | 1728 | 2281 |
| hRosa26 | E7 | GAGATCACGCGAGGAGGAAAGG | 1738 | 2282 |
| TRAC | G1 | ACCTTCTTCCCCAGCCCAGGTA | 1764 | 2283 |
| EMX1 | pPE641 | GGTCGTAGCCAGTCCGAACCC | 1809 | 2284 |
| Intron | | | | |
| AAVS1 | P2B1 | AGACTAGAGAGGTAAGGGGGGT | 1812 | 2285 |
| AAVS1 | P2B10 | TCTGTCCCCTCCACCCCACAGT | 1884 | 2286 |
| AAVS1 | P2C2 | TCCACAGGAGAACGGGGTGTCC | 1821 | 2287 |
| AAVS1 | P2C6 | CCTGTGCCATCTCTCGTTTCTT | 1853 | 2288 |
| AAVS1 | P2C11 | GGTCTAACCCCCACCTCCTGTT | 1893 | 2289 |
| AAVS1 | P2C12 | TGGAGCCAGAGAGGATCCTGGG | 1846 | 2290 |
| AAVS1 | P2D5 | GGCCATCCTAAGAAACGAGAGA | 1854 | 2291 |
| AAVS1 | P2D6 | TGCCATCTCTCGTTTCTTAGGA | 1878 | 2292 |
| AAVS1 | P2D9 | GACCACCTTATATTCCCAGGGC | 1886 | 2293 |
| AAVS1 | P2D10 | TCCACCCCACAGTGGGGCCACT | 1902 | 2294 |
| AAVS1 | P2D12 | GGAGCCAGAGAGGATCCTGGGA | 1890 | 2295 |
| AAVS1 | P2E5 | CCTAAGAAACGAGAGATGGCAC | 1847 | 2296 |
| AAVS1 | P2E12 | GAGGATCCTGGGAGGGAGAGCT | 1903 | 2297 |
| AAVS1 | P2H10 | GTGACAGAAAAGCCCCATCCTT | 1890 | 2298 |
| AAVS1 | MG34-25 C7 | ACCCAATATCAGGAGACTAGGA | 1957 | 2299 |
| AAVS1 | MG34-25 E7 | TATCAGGAGACTAGGAAGGAGG | 1959 | 2300 |
| AAVS1 | MG34-25 F7 | AGACTAGGAAGGAGGAGGCCTA | 1960 | 2301 |
| AAVS1 | MG34-25 G7 | TAGGAAGGAGGAGGCCTAAGGA | 1961 | 2302 |
| AAVS1 | MG34-25 E9 | ATTAACCGGCCCTGGGAATATA | 1975 | 2303 |
| AAVS1 | MG34-25 B11 | CCTGAAGTGGACATAGGGGCCC | 1988 | 2304 |
| AAVS1 | MG34-25 H12 | AGCACAGACTAGAGAGGTAAGG | 2002 | 2305 |

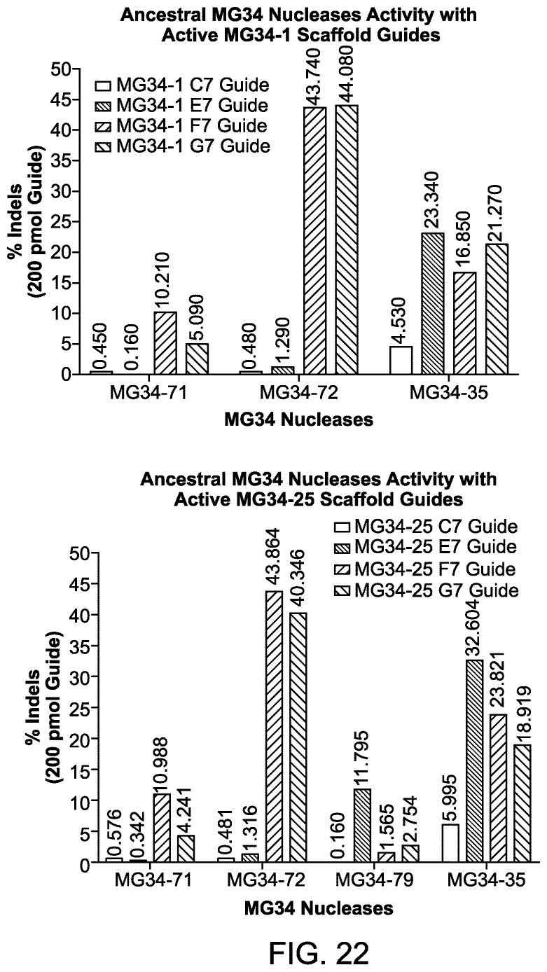

The ancestral nuclease MG34-71 (SEQ ID No. 1431) was found to be active with guides with the MG34-1 and MG34-25 guide backbones and NGG PAMs targeting AAV S1. 24 guides were tested and two (2) showed activity >5% indel formation, with maximum editing of 10% (FIG. 22, Table 11).

TABLE 11
MG34-71 guides with >5% indel formation at AAVS1

| | | | sgRNA | Target site in |
|---|---|---|---|---|
| | sgRNA | | SEQ ID | genome SEQ |
| Locus | well | Target site in genome | NO: | ID NO |
| AAVS1 | F7 | AGACTAGGAAGGAGGAGGCCTA | 1410 | 2301 |
| AAVS1 | MG34-25 F7 | AGACTAGGAAGGAGGAGGCCTA | 1960 | 2301 |

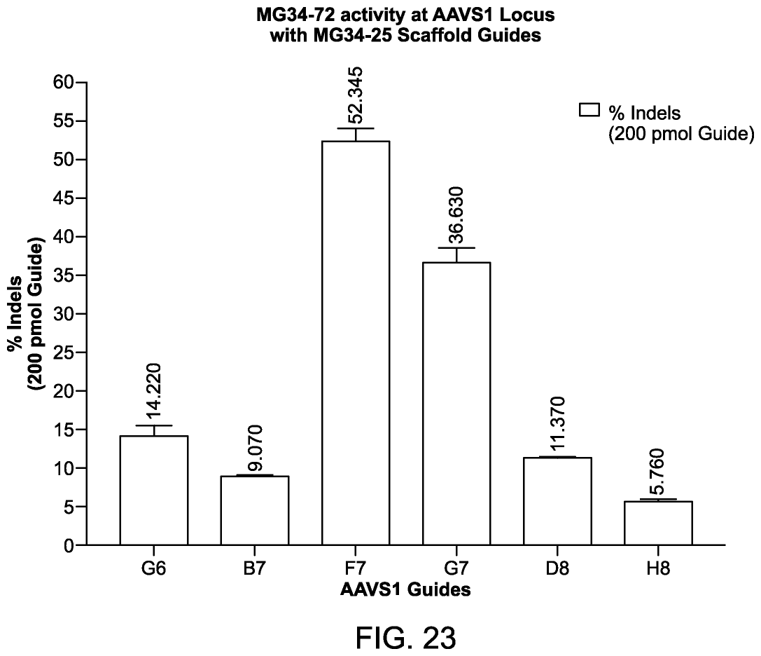

The ancestral nuclease MG34-72 (SEQ ID NO: 1432) was found to be active with guides with the MG34-1, MG34-25, and MG34-35 guide backbones and NGG PAMs targeting AAVS1. 120 guides were tested and nine (9) showed an activity of >5% indel formation, with an editing (% indels) of up to 52% (FIGS. 22 and 23, Table 12).

TABLE 12
MG34-72 guides with >5% indel formation at AAVS1

| | | | sgRNA | Target site |
|---|---|---|---|---|
| | sgRNA | | SEQ | in genome |
| Locus | well | Target site in genome | ID NO: | SEQ ID NO |
| AAVS1 | F7 | AGACTAGGAAGGAGGAGGCCTA | 1410 | 2301 |
| AAVS1 | G7 | TAGGAAGGAGGAGGCCTAAGGA | 1412 | 2302 |
| AAVS1 | MG34-25 G6 | GGAATCTGCCTAACAGGAGGTG | 1953 | 2306 |
| AAVS1 | MG34-25 B7 | TTAGACCCAATATCAGGAGACT | 1956 | 2307 |
| AAVS1 | MG34-25 F7 | AGACTAGGAAGGAGGAGGCCTA | 1960 | 2301 |
| AAVS1 | MG34-25 G7 | TAGGAAGGAGGAGGCCTAAGGA | 1961 | 2302 |
| AAVS1 | MG34-25 D8 | CTGTCCCTAGTGGCCCCACTGT | 1966 | 2308 |
| AAVS1 | MG34-25 H8 | AGTGGCCCCACTGTGGGGTGGA | 1970 | 2309 |
| AAVS1 | MG34-35 F7 | AGACTAGGAAGGAGGAGGCCTA | 1478 | 2301 |

The ancestral nuclease MG34-79 (SEQ ID No. 1439) was found to be active with guides with the MG34-25 guide backbone and NGG PAMs targeting AAVS1. 24 guides were tested and only one (1) showed an activity of >5% indel formation, with an editing (% indels) of up to 110% (FIG. 22, Table 13).

TABLE 13
MG34-79 guides with >5% indel formation at AAVS1

| | | | sgRNA | Target site in |
|---|---|---|---|---|
| | sgRNA | | SEQ ID | genome SEQ |
| Locus | well | Target site in genome | NO: | ID NO |
| AAVS1 | MG34-25 F7 | AGACTAGGAAGGAGGAGGCCTA | 1960 | 2301 |

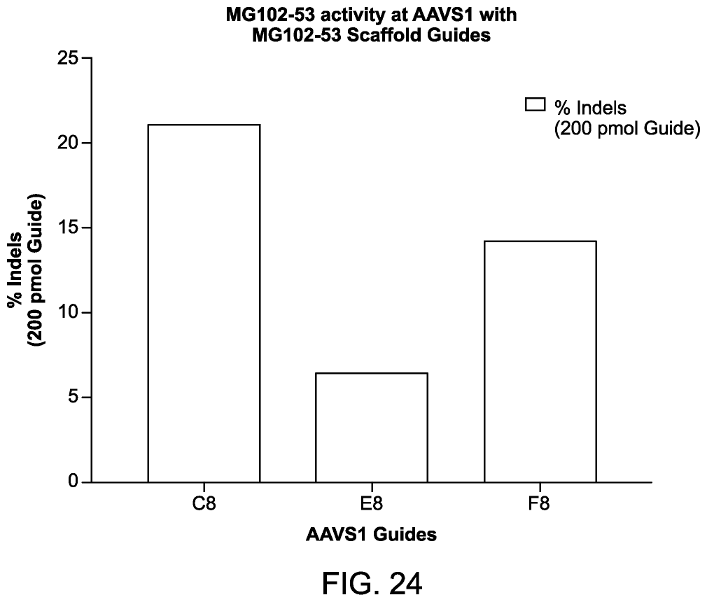

The native nuclease MG102-53 (SEQ ID No. 1264) was found to be active with guides with the MG102-53 guide backbone and NAR PAMs targeting AAVS1. 96 guides were tested and three (3) showed an activity of >5% indel formation, with an editing (% indels) of >20% (FIG. 24, Table 14).

TABLE 14
MG102-53 guides with >5% indel formation at AAVS1

| | | | sgRNA | Target site in |
|---|---|---|---|---|
| | sgRNA | | SEQ | genome SEQ ID |
| Locus | well | Target site in genome | ID NO: | NO |
| AAVS1 | MG102-53 C8 | TTAGACCCAATATCAGGAGACTAG | 2157 | 2310 |
| AAVS1 | MG102-53 E8 | ACCCAATATCAGGAGACTAGGAAG | 2159 | 2311 |
| AAVS1 | MG102-53 F8 | CAATATCAGGAGACTAGGAAGGAG | 2160 | 2312 |

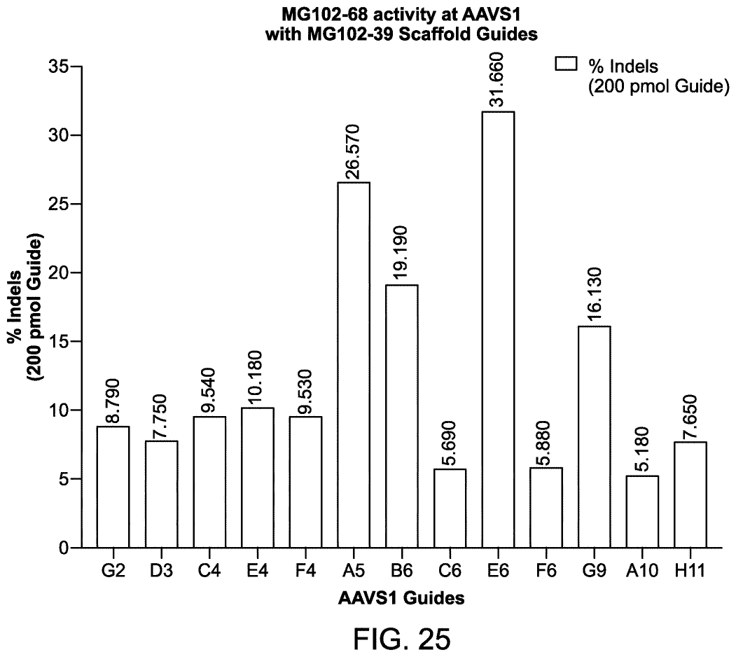

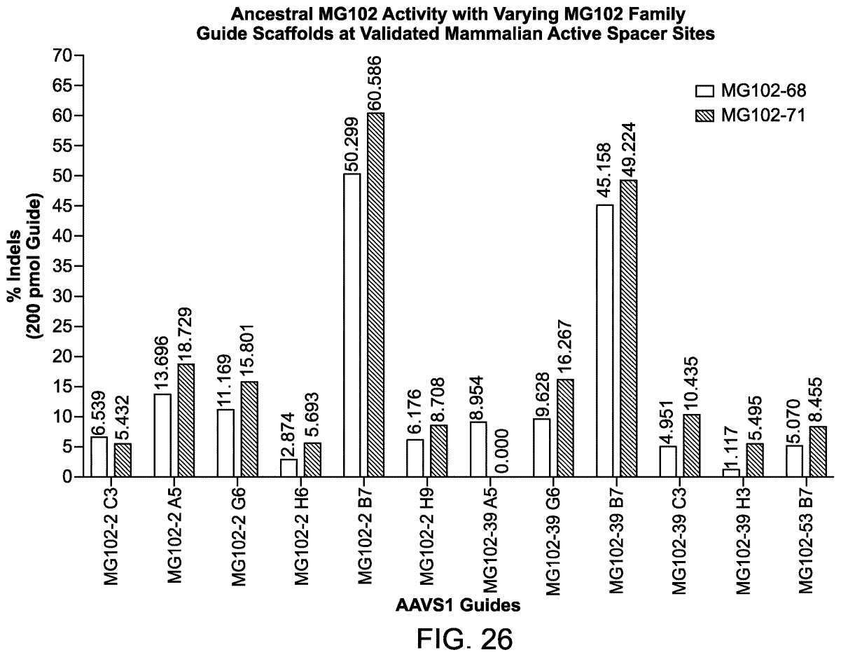

102971 The ancestral nuclease MG102-68 (SEQ ID No. 1333) was found to be active with guides with the MG102-39 guide backbone (FIG. 25), MG122-2 guide backbone (Table 15), and MG12-53 guide backbone (FIG. 26) with NRC PAMs targeting AAVS1. 120 guides were tested and twenty-five (25) showed activity >5%0 indel formation, with maximum editing of 50% (FIGS. 25 and 26, Table 15).

TABLE 15
MG102-68 guides with >5% indel formation at AAVS1

| | | | sgRNA | Target site in |
|---|---|---|---|---|
| | sgRNA | | SEQ | genome |
| Locus | well | Target site in genome | ID NO: | SEQ ID NO |
| AAVS1 | MG102-39 G2 | CCTCTTGGGAAGTGTAAGGA | 2017 | 2313 |
| AAVS1 | MG102-39 D3 | CAGCACCAGGATCAGTGAAA | 2022 | 2314 |
| AAVS1 | MG102-39 C4 | CTCAGGTTCTGGGAGAGGGT | 2029 | 2315 |
| AAVS1 | MG102-39 E4 | GGGAGAGGGTAGCGCAGGGT | 2031 | 2316 |
| AAVS1 | MG102-39 F4 | AGAGGGTAGCGCAGGGTGGC | 2032 | 2317 |
| AAVS1 | MG102-39 A5 | CCACTGAGAACCGGGCAGGT | 2035 | 2318 |
| AAVS1 | MG102-39 B6 | GTGTCACCAGATAAGGAATC | 2044 | 2319 |
| AAVS1 | MG102-39 C6 | ACCAGATAAGGAATCTGCCT | 2045 | 2320 |
| AAVS1 | MG102-39 E6 | CAGGAGACTAGGAAGGAGGA | 2047 | 2321 |
| AAVS1 | MG102-39 F6 | AGGAGGAGGCCTAAGGATGG | 2048 | 2322 |
| AAVS1 | MG102-39 G9 | GTGACCCGAATCCACAGGAG | 2073 | 2323 |
| AAVS1 | MG102-39 A10 | GAGGATGGAGAGGTGGCTAA | 2075 | 2324 |
| AAVS1 | MG102-39 H11 | CGGATGTCTCCCTTGCGTCC | 2090 | 2325 |
| AAVS1 | MG102-2 nRC C3 | TTCTCCTCTTGGGAAGTGTAAGGA | 2195 | 2326 |
| AAVS1 | MG102-2 nRC A5 | TTCTGGGAGAGGGTAGCGCAGGGT | 2197 | 2327 |
| AAVS1 | MG102-2 nRC G6 | TGGCTCCAGGAAATGGGGGTGTGT | 2198 | 2328 |
| AAVS1 | MG102-2 nRC H6 | GGGTGTGTCACCAGATAAGGAATC | 2199 | 2329 |
| AAVS1 | MG102-2 nRC B7 | CTGCCTAACAGGAGGTGGGGGTTA | 2200 | 2330 |
| AAVS1 | MG102-2 nRC H9 | ATGCTGTCCTGAAGTGGACATAGG | 2202 | 2331 |
| AAVS1 | MG102-39 nRC A5 | TTCTGGGAGAGGGTAGCGCAGGGT | 2205 | 2327 |
| AAVS1 | MG102-39 nRC G6 | TGGCTCCAGGAAATGGGGGTGTGT | 2206 | 2328 |
| AAVS1 | MG102-39 nRC B7 | CTGCCTAACAGGAGGTGGGGGTTA | 2208 | 2330 |
| AAVS1 | MG102-39 nRC C3 | TTCTCCTCTTGGGAAGTGTAAGGA | 2211 | 2326 |
| AAVS1 | MG102-39 nRC H3 | GCTGCAGCACCAGGATCAGTGAAA | 2212 | 2332 |
| AAVS1 | MG102-53 nRC B7 | CTGCCTAACAGGAGGTGGGGGTTA | 2216 | 2330 |

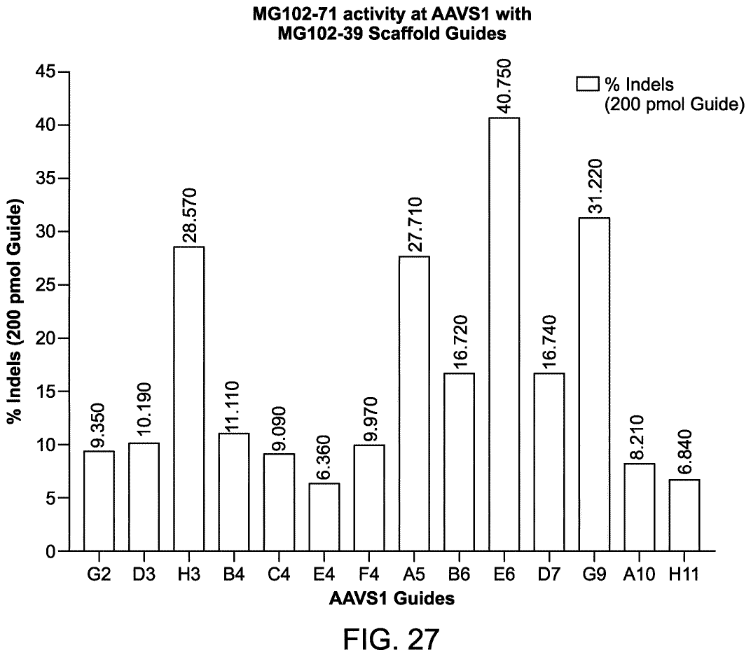

The ancestral nuclease MG102-71 (SEQ ID No. 1336) was found to be active with guides with the MG102-2, MG102-39, MG102-53 guide backbone and NRC PAMs targeting AAV S1. 120 guides were tested and twenty-five (25) showed an activity of >5% indel formation, with an editing (% indels) of up to 60% (FIGS. 26 and 27, Table 16).

TABLE 16
MG102-71 guides with >5% indel formation at AAVS1

| | | | sgRNA | Target site in |
|---|---|---|---|---|
| | | | SEQ | genome SEQ ID |
| Locus | sgRNA well | Target site in genome | ID NO: | NO |
| AAVS1 | MG102-39 G2 | CCTCTTGGGAAGTGTAAGGA | 2017 | 2313 |
| AAVS1 | MG102-39 D3 | CAGCACCAGGATCAGTGAAA | 2022 | 2314 |
| AAVS1 | MG102-39 H3 | AGTGAAACGCACCAGACGGC | 2026 | 2333 |
| AAVS1 | MG102-39 B4 | CCAGACGGCCGCGTCAGAGC | 2028 | 2334 |
| AAVS1 | MG102-39 C4 | CTCAGGTTCTGGGAGAGGGT | 2029 | 2315 |
| AAVS1 | MG102-39 E4 | GGGAGAGGGTAGCGCAGGGT | 2031 | 2316 |
| AAVS1 | MG102-39 F4 | AGAGGGTAGCGCAGGGTGGC | 2032 | 2317 |
| AAVS1 | MG102-39 A5 | CCACTGAGAACCGGGCAGGT | 2035 | 2318 |
| AAVS1 | MG102-39 B6 | GTGTCACCAGATAAGGAATC | 2044 | 2319 |
| AAVS1 | MG102-39 E6 | CAGGAGACTAGGAAGGAGGA | 2047 | 2321 |
| AAVS1 | MG102-39 D7 | CCCAGAACCAGAGCCACATT | 2054 | 2269 |
| AAVS1 | MG102-39 G9 | GTGACCCGAATCCACAGGAG | 2073 | 2323 |
| AAVS1 | MG102-39 A10 | GAGGATGGAGAGGTGGCTAA | 2075 | 2324 |
| AAVS1 | MG102-39 H11 | CGGATGTCTCCCTTGCGTCC | 2090 | 2325 |
| AAVS1 | MG102-2 nRC C3 | TTCTCCTCTTGGGAAGTGTAAGGA | 2195 | 2326 |
| AAVS1 | MG102-2 nRC A5 | TTCTGGGAGAGGGTAGCGCAGGGT | 2197 | 2327 |
| AAVS1 | MG102-2 nRC G6 | TGGCTCCAGGAAATGGGGGTGTGT | 2198 | 2328 |
| AAVS1 | MG102-2 nRC H6 | GGGTGTGTCACCAGATAAGGAATC | 2199 | 2329 |
| AAVS1 | MG102-2 nRC B7 | CTGCCTAACAGGAGGTGGGGGTTA | 2200 | 2330 |
| AAVS1 | MG102-2 nRC H9 | ATGCTGTCCTGAAGTGGACATAGG | 2202 | 2331 |
| AAVS1 | MG102-39 nRC G6 | TGGCTCCAGGAAATGGGGGTGTGT | 2206 | 2328 |
| AAVS1 | MG102-39 nRC B7 | CTGCCTAACAGGAGGTGGGGGTTA | 2208 | 2330 |
| AAVS1 | MG102-39 nRC C3 | TTCTCCTCTTGGGAAGTGTAAGGA | 2211 | 2326 |
| AAVS1 | MG102-39 nRC H3 | GCTGCAGCACCAGGATCAGTGAAA | 2212 | 2332 |
| AAVS1 | MG102-53 nRC B7 | CTGCCTAACAGGAGGTGGGGGTTA | 2216 | 2330 |

### REFERENCES

1. Eddy S R. Accelerated Profile HMM Searches. Plos Comput Biol 7: e1002195. doi:10.1371/joumnal.pcbi.1002195 (2011).
2. Charles Bland et al., CRISPR Recognition Tool (CRT): a tool for automatic detection of clustered regularly interspaced palindromic repeats, BMC Bioinformatics 8, no. 1 (2007): 209.
3. Katoh K, Standley D M. MAFFT multiple sequence alignment software version 7: improvements in performance and usability. Mol Biol Evol. 2013; 30(4):772-780. doi:10.1093/molbev/mst010.
4. Price, M. N., Dehal, P. S., and Arkin, A. P. FastTree 2—Approximately Maximum-Likelihood Trees for Large Alignments. PLoS ONE, 5(3): e9490 (2010).
5. Stamatakis, A. RAxML version 8: a tool for phylogenetic analysis and post-analysis of large phylogenies. Bioinformatics 30(9), 1312-1313 (2014).
6. Yang, Z., PAML 4: a program package for phylogenetic analysis by maximum likelihood. Molecular Biology and Evolution 24: 1586-1591 (2007).
7. Tareen, A. & Kinney, J. B. Logomaker: beautiful sequence logos in Python. Bioinformatics 36, 2272-2274 (2020).

While embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all embodiments of the disclosure are not limited to the specific depictions, configurations or relative proportions set forth herein which depend upon a variety of conditions and variables. It should be understood that various alternatives to the embodiments of the disclosure described herein, in some embodiment, are be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

TABLE 17
Listing and/or Information of protein and nucleic acid sequences referred to herein

| | SEQ ID | | | | | |
|---|---|---|---|---|---|---|
| Category | NO: | Description | Type | Organism | Other Information | Sequence |
| MG102 effector | 1322 | MG102-82 effector | protein | unknown | uncultivated organism | |
| MG102 effector | 1323 | MG102-83 effector | protein | unknown | uncultivated organism | |
| MG102 effector | 1324 | MG102-63 effector | protein | unknown | uncultivated organism | |
| MG102 predicted CRISPR | 1325 | MG102-83 CRISPR repeat | nucleotide | artificial sequence | | |
| repeat | | | | | | |
| MG102 predicted CRISPR | 1326 | MG102-63 CRISPR repeat | nucleotide | artificial sequence | | |
| repeat | | | | | | |
| MG102 effector tracrRNA | 1327 | MG102-83 tracrRNA | nucleotide | artificial sequence | | |
| MG102 effector tracrRNA | 1328 | MG102-63 tracrRNA | nucleotide | artificial sequence | | |
| MG102 effector | 1329 | MG102-64 effector | protein | unknown | uncultivated organism | |
| MG102 effector | 1330 | MG102-65 effector | protein | unknown | uncultivated organism | |
| MG102 effector | 1331 | MG102-66 effector | protein | unknown | uncultivated organism | |
| MG102 effector | 1332 | MG102-67 effector | protein | unknown | uncultivated organism | |
| MG102 effector | 1333 | MG102-68 effector | protein | unknown | uncultivated organism | |
| MG102 effector | 1334 | MG102-69 effector | protein | unknown | uncultivated organism | |
| MG102 effector | 1335 | MG102-70 effector | protein | unknown | uncultivated organism | |
| MG102 effector | 1336 | MG102-71 effector | protein | unknown | uncultivated organism | |
| MG102 effector | 1337 | MG102-72 effector | protein | unknown | uncultivated organism | |
| MG102 effector | 1338 | MG102-73 effector | protein | unknown | uncultivated organism | |
| MG102 effector | 1339 | MG102-74 effector | protein | unknown | uncultivated organism | |
| MG102 effector | 1340 | MG102-75 effector | protein | unknown | uncultivated organism | |
| MG102 effector | 1341 | MG102-76 effector | protein | unknown | uncultivated organism | |
| MG102 effector | 1342 | MG102-77 effector | protein | unknown | uncultivated organism | |
| MG102 effector | 1343 | MG102-78 effector | protein | unknown | uncultivated organism | |
| MG102 effector | 1344 | MG102-79 effector | protein | unknown | uncultivated organism | |
| MG102 effector | 1345 | MG102-80 effector | protein | unknown | uncultivated organism | |
| MG102 effector | 1346 | MG102-81 effector | protein | unknown | uncultivated organism | |
| MG34 effector | 1347 | MG34-35 effector | protein | unknown | uncultivated organism | |
| MG34 effector tracrRNA | 1348 | MG34-35 tracrRNA | nucleotide | artificial sequence | | |
| MG34 predicted CRISPR | 1349 | MG34-35 CRISPR repeat | nucleotide | artificial sequence | | |
| repeat | | | | | | |
| MG34 effector | 1350 | MG34-36 effector | protein | unknown | uncultivated organism | |
| MG34 effector | 1351 | MG34-37 effector | protein | unknown | uncultivated organism | |
| MG34 effector | 1352 | MG34-38 effector | protein | unknown | uncultivated organism | |
| MG34 effector | 1353 | MG34-39 effector | protein | unknown | uncultivated organism | |
| MG34 effector | 1354 | MG34-40 effector | protein | unknown | uncultivated organism | |
| MG34 effector | 1355 | MG34-41 effector | protein | unknown | uncultivated organism | |
| MG34 effector | 1356 | MG34-42 effector | protein | unknown | uncultivated organism | |
| MG34 effector | 1357 | MG34-43 effector | protein | unknown | uncultivated organism | |
| MG34 effector | 1358 | MG34-44 effector | protein | unknown | uncultivated organism | |
| MG34 effector | 1359 | MG34-45 effector | protein | unknown | uncultivated organism | |
| MG34 effector | 1360 | MG34-46 effector | protein | unknown | uncultivated organism | |
| MG34 effector | 1361 | MG34-47 effector | protein | unknown | uncultivated organism | |
| MG34 effector | 1362 | MG34-48 effector | protein | unknown | uncultivated organism | |
| MG34 effector | 1363 | MG34-49 effector | protein | unknown | uncultivated organism | |
| MG34 effector | 1364 | MG34-50 effector | protein | unknown | uncultivated organism | |
| MG34 effector | 1365 | MG34-51 effector | protein | unknown | uncultivated organism | |
| MG34 effector | 1366 | MG34-52 effector | protein | unknown | uncultivated organism | |
| MG34 effector | 1367 | MG34-53 effector | protein | unknown | uncultivated organism | |
| MG34 effector | 1368 | MG34-54 effector | protein | unknown | uncultivated organism | |
| MG34 effector sgRNA | 1369 | MG34 sg25 single guide | nucleotide | artificial sequence | | |
| | | RNA | | | | |
| MG102 effector tracrRNA | 1370 | MG102-51 tracrRNA | nucleotide | artificial sequence | | |
| MG102 effector tracrRNA | 1371 | MG102-53 tracrRNA | nucleotide | artificial sequence | | |
| MG102 effector tracrRNA | 1372 | MG102-55 tracrRNA | nucleotide | artificial sequence | | |
| MG102 predicted CRISPR | 1373 | MG102-51 CRISPR repeat | nucleotide | artificial sequence | | |
| repeat | | | | | | |
| MG102 predicted CRISPR | 1374 | MG102-53 CRISPR repeat | nucleotide | artificial sequence | | |
| repeat | | | | | | |
| MG102 predicted CRISPR | 1375 | MG102-55 CRISPR repeat | nucleotide | artificial sequence | | |
| repeat | | | | | | |
| MG102 effector sgRNA | 1376 | MG102-51 sg1 targeting | nucleotide | artificial sequence | | |
| | | U67 20 nt | | | | |
| MG102 effector sgRNA | 1377 | MG102-51 sgl targeting | nucleotide | artificial sequence | | |
| | | U67 24 nt | | | | |
| MG102 effector sgRNA | 1378 | MG102-51 sg2 targeting | nucleotide | artificial sequence | | |
| | | U67 20 nt | | | | |
| MG102 effector sgRNA | 1379 | MG102-51 sg2 targeting | nucleotide | artificial sequence | | |
| | | U67 24 nt | | | | |
| MG102 effector sgRNA | 1380 | MG102-53 sg1 targeting | nucleotide | artificial sequence | | |
| | | U67 20 nt | | | | |
| MG102 effector sgRNA | 1381 | MG102-53 sg1 targeting | nucleotide | artificial sequence | | |
| | | U67 24 nt | | | | |
| MG102 effector sgRNA | 1382 | MG102-53 sg2 targeting | nucleotide | artificial sequence | | |
| | | U67 20 nt | | | | |
| MG102 effector sgRNA | 1383 | MG102-53 sg2 targeting | nucleotide | artificial sequence | | |
| | | U67 24 nt | | | | |
| MG102 effector sgRNA | 1384 | MG102-55 sg1 targeting | nucleotide | artificial sequence | | |
| | | U67 20 nt | | | | |
| MG102 effector sgRNA | 1385 | MG102-55 sg1 targeting | nucleotide | artificial sequence | | |
| | | U67 24 nt | | | | |
| MG102 effector sgRNA | 1386 | MG102-55 sg2 targeting | nucleotide | artificial sequence | | |
| | | U67 20 nt | | | | |
| MG102 effector sgRNA | 1387 | MG102-55 sg2 targeting | nucleotide | artificial sequence | | |
| | | U67 24 nt | | | | |
| MG102 effector sgRNA | 1388 | MG102-63 sg1 targeting | nucleotide | artificial sequence | | |
| | | U67 20 nt | | | | |
| MG102 effector sgRNA | 1389 | MG102-63 sg1 targeting | nucleotide | artificial sequence | | |
| | | U67 24 nt | | | | |
| MG102 effector sgRNA | 1390 | MG102-63 sg2 targeting | nucleotide | artificial sequence | | |
| | | U67 20 nt | | | | |
| MG102 effector sgRNA | 1391 | MG102-63 sg2 targeting | nucleotide | artificial sequence | | |
| | | U67 24 nt | | | | |
| MG34 effector sgRNA | 1392 | MG34-35 sg1 targeting | nucleotide | artificial sequence | | |
| | | U67 24 nt | | | | |
| MG34 effector sgRNA | 1393 | MG34-35 sg1 targeting | nucleotide | artificial sequence | | |
| | | U67 20 nt | | | | |
| MG34 effector sgRNA | 1394 | MG34-35 sg2 targeting | nucleotide | artificial sequence | | |
| | | U67 20 nt | | | | |
| MG34 effector sgRNA | 1395 | MG34-35 sg3 targeting | nucleotide | artificial sequence | | |
| | | U67 20 nt | | | | |
| MG34 effector sgRNA | 1396 | MG34-35 sg1 targeting | nucleotide | artificial sequence | | |
| | | U40 24 nt | | | | |
| MG34 effector sgRNA | 1397 | MG34-35 sg1 targeting | nucleotide | artificial sequence | | |
| | | U40 20 nt | | | | |
| MG34 effector sgRNA | 1398 | MG34-35 sg2 targeting | nucleotide | artificial sequence | | |
| | | U40 20 nt | | | | |
| MG34 effector sgRNA | 1399 | MG34-35 sg3 targeting | nucleotide | artificial sequence | | |
| | | U40 20 nt | | | | |
| MG34 effector chemically | 1400 | MG34-1 sgRNA targeting | nucleotide | artificial sequence | | |
| synthesized/modified sgRNA | | AAVS1 B7 | | | | |
| MG34 effector chemically | 1401 | MG34-1 sgRNA targeting | nucleotide | artificial sequence | | |
| synthesized/modified sgRNA | | AAVS1 B11 | | | | |
| MG34 effector chemically | 1402 | MG34-1 sgRNA targeting | nucleotide | artificial sequence | | |
| synthesized/modified sgRNA | | AAVS1 C7 | | | | |
| MG34 effector chemically | 1403 | MG34-1 sgRNA targeting | nucleotide | artificial sequence | | |
| synthesized/modified sgRNA | | AAVS1 C11 | | | | |
| MG34 effector chemically | 1404 | MG34-1 sgRNA targeting | nucleotide | artificial sequence | | |
| synthesized/modified sgRNA | | AAVS1 D5 | | | | |
| MG34 effector chemically | 1405 | MG34-1 sgRNA targeting | nucleotide | artificial sequence | | |
| synthesized/modified sgRNA | | AAVS1 D8 | | | | |
| MG34 effector chemically | 1406 | MG34-1 sgRNA targeting | nucleotide | artificial sequence | | |
| synthesized/modified sgRNA | | AAVS1 E6 | | | | |
| MG34 effector chemically | 1407 | MG34-1 sgRNA targeting | nucleotide | artificial sequence | | |
| synthesized/modified sgRNA | | AAVS1 E7 | | | | |
| MG34 effector chemically | 1408 | MG34-1 sgRNA targeting | nucleotide | artificial sequence | | |
| synthesized/modified sgRNA | | AAVS1 E8 | | | | |
| MG34 effector chemically | 1409 | MG34-1 sgRNA targeting | nucleotide | artificial sequence | | |
| synthesized/modified sgRNA | | AAVS1 E9 | | | | |
| MG34 effector chemically | 1410 | MG34-1 sgRNA targeting | nucleotide | artificial sequence | | |
| synthesized/modified sgRNA | | AAVS1 F7 | | | | |
| MG34 effector chemically | 1411 | MG34-1 sgRNA targeting | nucleotide | artificial sequence | | |
| synthesized/modified sgRNA | | AAVS1 G6 | | | | |
| MG34 effector chemically | 1412 | MG34-1 sgRNA targeting | nucleotide | artificial sequence | | |
| synthesized/modified sgRNA | | AAVS1 G7 | | | | |
| MG34 effector chemically | 1413 | MG34-1 sgRNA targeting | nucleotide | artificial sequence | | |
| synthesized/modified sgRNA | | AAVS1 H6 | | | | |
| MG34 effector chemically | 1414 | MG34-1 sgRNA targeting | nucleotide | artificial sequence | | |
| synthesized/modified sgRNA | | AAVS1 H12 | | | | |
| MG34 effector | 1415 | MG34-55 effector | protein | unknown | uncultivated organism | |
| MG34 effector | 1416 | MG34-56 effector | protein | unknown | uncultivated organism | |
| MG34 effector | 1417 | MG34-57 effector | protein | unknown | uncultivated organism | |
| MG34 effector | 1418 | MG34-58 effector | protein | unknown | uncultivated organism | |
| MG34 effector | 1419 | MG34-59 effector | protein | unknown | uncultivated organism | |
| MG34 effector | 1420 | MG34-60 effector | protein | unknown | uncultivated organism | |
| MG34 effector | 1421 | MG34-61 effector | protein | unknown | uncultivated organism | |
| MG34 effector | 1422 | MG34-62 effector | protein | unknown | uncultivated organism | |
| MG34 effector | 1423 | MG34-63 effector | protein | unknown | uncultivated organism | |
| MG34 effector | 1424 | MG34-64 effector | protein | unknown | uncultivated organism | |
| MG34 effector | 1425 | MG34-65 effector | protein | unknown | uncultivated organism | |
| MG34 effector | 1426 | MG34-66 effector | protein | unknown | uncultivated organism | |
| MG34 effector | 1427 | MG34-67 effector | protein | unknown | uncultivated organism | |
| MG34 effector | 1428 | MG34-68 effector | protein | unknown | uncultivated organism | |
| MG34 effector | 1429 | MG34-69 effector | protein | unknown | uncultivated organism | |
| MG34 effector | 1430 | MG34-70 effector | protein | unknown | uncultivated organism | |
| MG34 effector | 1431 | MG34-71 effector | protein | unknown | uncultivated organism | |
| MG34 effector | 1432 | MG34-72 effector | protein | unknown | uncultivated organism | |
| MG34 effector | 1433 | MG34-73 effector | protein | unknown | uncultivated organism | |
| MG34 effector | 1434 | MG34-74 effector | protein | unknown | uncultivated organism | |
| MG34 effector | 1435 | MG34-75 effector | protein | unknown | uncultivated organism | |
| MG34 effector | 1436 | MG34-76 effector | protein | unknown | uncultivated organism | |
| MG34 effector | 1437 | MG34-77 effector | protein | unknown | uncultivated organism | |
| MG34 effector | 1438 | MG34-78 effector | protein | unknown | uncultivated organism | |
| MG34 effector | 1439 | MG34-79 effector | protein | unknown | uncultivated organism | |
| MG34 effector | 1440 | MG34-80 effector | protein | unknown | uncultivated organism | |
| MG34 effector PAM | 1441 | MG34-35 PAM | nucleotide | | | NRG |
| MG34 effector PAM | 1442 | MG34-71 PAM | nucleotide | | | NGG |
| MG34 effector PAM | 1443 | MG34-72 PAM | nucleotide | | | NGG |
| MG34 effector PAM | 1444 | MG34-73 PAM | nucleotide | | | NGGT |
| MG34 effector PAM | 1445 | MG34-74 PAM | nucleotide | | | NGGT |
| MG34 effector PAM | 1446 | MG34-75 PAM | nucleotide | | | NGGT |
| MG34 effector PAM | 1447 | MG34-76 PAM | nucleotide | | | NGGT |
| MG34 effector PAM | 1448 | MG34-77 PAM | nucleotide | | | NGGTNNA |
| MG34 effector PAM | 1449 | MG34-78 PAM | nucleotide | | | NGG |
| MG34 effector PAM | 1450 | MG34-79 PAM | nucleotide | | | NGG |
| MG102 effector PAM | 1451 | MG102-51 PAM | nucleotide | | | NAR |
| MG102 effector PAM | 1452 | MG102-53 PAM | nucleotide | | | NAR |
| MG102 effector PAM | 1453 | MG102-55 PAM | nucleotide | | | NRMC |
| MG102 effector PAM | 1454 | MG102-63 PAM | nucleotide | | | WRC |
| MG102 effector PAM | 1455 | MG102-65 PAM with | nucleotide | | | RAR |
| | | MG102-2_sg | | | | |
| MG102 effector PAM | 1456 | MG102-65 PAM with | nucleotide | | | NRM |
| | | MG102-39_sg | | | | |
| MG102 effector PAM | 1457 | MG102-66 PAM | nucleotide | | | NRC |
| MG102 effector PAM | 1458 | MG102-67 PAM with | nucleotide | | | NAMC |
| | | MG102-2_sg | | | | |
| MG102 effector PAM | 1459 | MG102-67 PAM with | nucleotide | | | NRM |
| | | MG102-39_sg | | | | |
| MG102 effector PAM | 1460 | MG102-68 PAM | nucleotide | | | NRC |
| MG102 effector PAM | 1461 | MG102-71 PAM | nucleotide | | | NRC |
| MG102 effector PAM | 1462 | MG102-73 PAM | nucleotide | | | NRM |
| MG102 effector PAM | 1463 | MG102-74 PAM | nucleotide | | | NRCNT |
| MG102 effector PAM | 1464 | MG102-77 PAM | nucleotide | | | CRMS |
| MG102 effector PAM | 1465 | MG102-78 PAM | nucleotide | | | NRM |
| MG102 effector PAM | 1466 | MG102-79 PAM | nucleotide | | | NRC |
| MG102 effector PAM | 1467 | MG102-80 PAM with | nucleotide | | | NAM |
| | | MG102-2_sg | | | | |
| MG102 effector PAM | 1468 | MG102-80 PAM with | nucleotide | | | NRC |
| | | MG102-39_sg | | | | |
| MG102 effector PAM | 1469 | MG102-80 PAM with | nucleotide | | | NACNT |
| | | MG102-42_sg | | | | |

1. An engineered nuclease system, comprising:
a) an endonuclease comprising a sequence having at least 80% sequence identity to any one of SEQ ID NOs: 1336, 1432, 1347, 1323, 1324, 1329-1335, 1337-1346, 1350-1368, 1415-1431, and 1433-1440; and
b) an engineered guide polynucleotide which forms a complex with the endonuclease and hybridizes to a target nucleic acid sequence.

2. The engineered nuclease system of claim 1, wherein the endonuclease comprises a sequence having at least 80% identity to any one of SEQ ID NOs: 1432, 1347, 1350-1368, 1415-1431, and 1433-1440.

3. The engineered nuclease system of claim 1, wherein the endonuclease comprises a sequence having at least 80% identity to any one of SEQ ID NOs: 1336, 1323, 1324, 1329-1335, and 1337-1346.

4-15. (canceled)

16. The engineered nuclease system of claim 1, wherein:
a) the engineered guide polynucleotide is a single guide nucleic acid, a dual guide nucleic acid, or RNA; and/or
b) the endonuclease binds non-covalently to the engineered guide polynucleotide, the endonuclease is covalently linked to the engineered guide polynucleotide, or the endonuclease is fused to the engineered guide polynucleotide.

17-21. (canceled)

22. The engineered nuclease system of claim 1, wherein the engineered guide polynucleotide comprises a sequence having at least 90% sequence identity to any one of SEQ ID NOs: 1327-1328, 1348, 1369-1372, 1376-1391, 1392-1414, and 1470-2242.

23. The engineered nuclease system of claim 1, wherein the engineered guide polynucleotide comprises a sequence having at least 90% sequence identity to any one of SEQ ID NOs: 1571, 1591, 1592, 1615, 1625, 1651, 1663, 1672, 1709, 1712, 1713, 1728, 1738, 1764, 1809, 1812, 1884, 1821, 1853, 1893, 1846, 1854, 1878, 1886, 1902, 1890, 1847, 1903, 1890, 1957, 1959, 1960, 1961, 1975, 1988, and 2002.

24. The engineered nuclease system of claim 1, wherein the engineered guide polynucleotide comprises a sequence having at least 90% sequence identity to SEQ ID NOs: 1410 or 1960.

25. The engineered nuclease system of claim 1, wherein the engineered guide polynucleotide comprises a sequence having at least 90% sequence identity to any one of SEQ ID NOs: 1410, 1412, 1953, 1956, 1960, 1961, 1966, 1970, and 1478.

26. The engineered nuclease system of claim 1, wherein the engineered guide polynucleotide comprises a sequence having at least 90% sequence identity to any one of SEQ ID NOs: 2157, 2159, and 2160.

27. The engineered nuclease system of claim 1, wherein the engineered guide polynucleotide comprises a sequence having at least 90% sequence identity to any one of SEQ ID NOs: 2017, 2022, 2029, 2031, 2032, 2035, 2044, 2045, 2047, 2048, 2073, 2075, 2090, 2195, 2197, 2198, 2199, 2200, and 2202.

28. The engineered nuclease system of claim 1, wherein the engineered guide polynucleotide comprises a sequence having at least 90% sequence identity to any one of SEQ ID NOs: 2017, 2022, 2026, 2028, 2029, 2031, 2032, 2035, 2044, 2047, 2054, 2073, 2075, 2090, 2195, 2197, 2198, 2199, 2200, 2202, 2206, 2208, 2211, 2212, and 2216.

29. The engineered nuclease system of claim 1, wherein the engineered guide polynucleotide comprises a sequence having 100% sequence identity to any one of SEQ ID NOs: 1327-1328, 1348, 1369-1372, 1376-1391, 1392-1414, and 1470-2242.

30. A method for modifying a target nucleic acid sequence comprising contacting the target nucleic acid sequence using the engineered nuclease system of claim 1.

31. The method of claim 30, wherein modifying the target nucleic acid sequence comprises binding, nicking, or cleaving the target nucleic acid sequence.

32. The method of claim 30, wherein the target nucleic acid sequence comprises genomic DNA, viral DNA, viral RNA, or bacterial DNA.

33. The method of claim 30, wherein the modification is in vitro, in vivo, or ex vivo.

34-35. (canceled)

36. A method of modifying a target nucleic acid sequence in a mammalian cell comprising contacting the mammalian cell using the engineered nuclease system of claim 1.

37. The method of claim 36, further comprising selecting cells comprising the modification.

38. A cell comprising the engineered nuclease system of claim 1.

39. The cell of claim 38, wherein the cell is:
a) a eukaryotic cell;
b) a mammalian cell;
c) an immortalized cell;
d) an insect cell;
e) a yeast cell;
f) a plant cell;
g) a fungal cell;
h) a prokaryotic cell;
i) an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof;
j) an engineered cell;
k) a stable cell; or
l) a primary cell.

40-49. (canceled)
